SEQUENCE LISTING (86 sequences)

SEQ ID NO: 1 - DNA, 19 bp, Artificial Sequence
  Artificially synthesized oligonucleotide primer
aattccagaaccgctccag

SEQ ID NO: 2 - DNA, 20 bp, Artificial Sequence
  Artificially synthesized oligonucleotide primer
ttgatgcagcctgagtgtct

SEQ ID NO: 3 - DNA, 21 bp, Artificial Sequence
  Artificially synthesized oligonucleotide primer
aggtcatcccagagctgaacg

SEQ ID NO: 4 - DNA, 21 bp, Artificial Sequence
  Artificially synthesized oligonucleotide primer
accctgttgctgtagccgtat

SEQ ID NO: 5 - DNA, 21 bp, Artificial Sequence
  Artificially synthesized oligonucleotide primer
acggccaggtcatcactattc

SEQ ID NO: 6 - DNA, 20 bp, Artificial Sequence
  Artificially synthesized oligonucleotide primer
aggaaggctggaaaagagcc

SEQ ID NO: 7 - DNA, 20 bp, Artificial Sequence
  Artificially synthesized oligonucleotide primer
tctatggcccagaccctcac

SEQ ID NO: 8 - DNA, 20 bp, Artificial Sequence
  Artificially synthesized oligonucleotide primer
gacggcagagaggaggttga

SEQ ID NO: 9 - DNA, 23 bp, Artificial Sequence
  Artificially synthesized oligonucleotide primer
ttgaggtcgtccaacttccagca

SEQ ID NO: 10 - DNA, 20 bp, Artificial Sequence
  Artificially synthesized oligonucleotide primer
agccggacgtcgtgttgtta

SEQ ID NO: 11 - DNA, 21 bp, Artificial Sequence
  Artificially synthesized oligonucleotide primer
tttaactcccttggcgcaaaa

SEQ ID NO: 12 - DNA, 20 bp, Artificial Sequence
  Artificially synthesized oligonucleotide primer
ctttccctccgcattgacac

SEQ ID NO: 13 - DNA, 23 bp, Artificial Sequence
  Artificially synthesized oligonucleotide primer
agccacccgctcgcttctctgtg

SEQ ID NO: 14 - DNA, 23 bp, Artificial Sequence
  Artificially synthesized oligonucleotide primer
agcctcgcgattcttgagtgtgg

SEQ ID NO: 15 - DNA, 20 bp, Artificial Sequence
  Artificially synthesized oligonucleotide primer
ctgtgcccttcagattgttg

SEQ ID NO: 16 - DNA, 20 bp, Artificial Sequence
  Artificially synthesized oligonucleotide primer
tcagccttcctcgggggtct

SEQ ID NO: 17 - DNA, 19 bp, Artificial Sequence
  Artificially synthesized oligonucleotide primer
gttggctcagccagatgca

SEQ ID NO: 18 - DNA, 24 bp, Artificial Sequence
  Artificially synthesized oligonucleotide primer
agcctactcattgggatcatcttg

SEQ ID NO: 19 - DNA, 21 bp, Artificial Sequence
  Artificially synthesized oligonucleotide primer
tttgaattccctgggtgagaa

SEQ ID NO: 20 - DNA, 20 bp, Artificial Sequence
  Artificially synthesized oligonucleotide primer
ggagaaatcgatgacagcgc

SEQ ID NO: 21 - DNA, 24 bp, Artificial Sequence
  Artificially synthesized oligonucleotide primer
tcagcaacagcaaggcgaaaaagg

SEQ ID NO: 22 - DNA, 20 bp, Artificial Sequence
  Artificially synthesized oligonucleotide primer
ccaccccgaatcagcagcga

SEQ ID NO: 23 - DNA, 21 bp, Artificial Sequence
  Artificially synthesized oligonucleotide primer
gtgggtttcacaagacagatg

SEQ ID NO: 24 - DNA, 21 bp, Artificial Sequence
  Artificially synthesized oligonucleotide primer
ttttcacccagttctgctttg

SEQ ID NO: 25 - DNA, 20 bp, Artificial Sequence
  Artificially synthesized oligonucleotide primer
gctttttgcctgcctggttg

SEQ ID NO: 26 - DNA, 20 bp, Artificial Sequence
  Artificially synthesized oligonucleotide primer
tcagtctgagagtctgaggc

SEQ ID NO: 27 - DNA, 24 bp, Artificial Sequence
  Artificially synthesized oligonucleotide primer
gagaggagacttcacagaggatac

SEQ ID NO: 28 - DNA, 20 bp, Artificial Sequence
  Artificially synthesized oligonucleotide primer
gtactccagaagaccagagg

SEQ ID NO: 29 - DNA, 20 bp, Artificial Sequence
  Artificially synthesized oligonucleotide primer
gagactctttcgggcttttc

SEQ ID NO: 30 - DNA, 21 bp, Artificial Sequence
  Artificially synthesized oligonucleotide primer
tgatgctctttaggctttcca

SEQ ID NO: 31 - DNA, 18 bp, Artificial Sequence
  Artificially synthesized oligonucleotide primer
ggtggctcagggaaatgt

SEQ ID NO: 32 - DNA, 20 bp, Artificial Sequence
  Artificially synthesized oligonucleotide primer
gacagagcaggcaggtacag

SEQ ID NO: 33 - DNA, 18 bp, Artificial Sequence
  Artificially synthesized oligonucleotide primer
cgctgtcaatgcctgaag

SEQ ID NO: 34 - DNA, 19 bp, Artificial Sequence
  Artificially synthesized oligonucleotide primer
ggcgtcacactcaagctct

SEQ ID NO: 35 - DNA, 20 bp, Artificial Sequence
  Artificially synthesized oligonucleotide primer
cccacgtgttgagatcattg

SEQ ID NO: 36 - DNA, 17 bp, Artificial Sequence
  Artificially synthesized oligonucleotide primer
tccatcacagcaccggg

SEQ ID NO: 37 - DNA, 29 bp, Artificial Sequence
  Artificially synthesized oligonucleotide primer
tactagagagacttcttccacaacaagag

SEQ ID NO: 38 - DNA, 27 bp, Artificial Sequence
  Artificially synthesized oligonucleotide primer
tctggtacatcttcaagtcctcataga

SEQ ID NO: 39 - DNA, 28 bp, Artificial Sequence
  Artificially synthesized oligonucleotide primer
gaccatcactgtcaaagagtttctagat

SEQ ID NO: 40 - DNA, 29 bp, Artificial Sequence
  Artificially synthesized oligonucleotide primer
aggaaagtcttgtttttgaaattttttaa

SEQ ID NO: 41 - DNA, 23 bp, Artificial Sequence
  Artificially synthesized oligonucleotide primer
cctcacattcttaggactggagc

SEQ ID NO: 42 - DNA, 19 bp, Artificial Sequence
  Artificially synthesized oligonucleotide primer
ggcaatcagagctctcgga

SEQ ID NO: 43 - DNA, 20 bp, Artificial Sequence
  Artificially synthesized oligonucleotide primer
cacagacttcacaagcccta

SEQ ID NO: 44 - DNA, 20 bp, Artificial Sequence
  Artificially synthesized oligonucleotide primer
gtacaagggtgggaggaaat

SEQ ID NO: 45 - DNA, 22 bp, Artificial Sequence
  Artificially synthesized oligonucleotide primer
tgacgtcactggagttgtacgg

SEQ ID NO: 46 - DNA, 21 bp, Artificial Sequence
  Artificially synthesized oligonucleotide primer
ggttcatgtcatggatggtgc

SEQ ID NO: 47 - DNA, 19 bp, Artificial Sequence
  Artificially synthesized oligonucleotide primer
atgcggattgccaaggagg

SEQ ID NO: 48 - DNA, 22 bp, Artificial Sequence
  Artificially synthesized oligonucleotide primer
tgaagacagctgctacaagtcc

SEQ ID NO: 49 - DNA, 20 bp, Artificial Sequence
  Artificially synthesized oligonucleotide primer
aacagagcagtgcgcatgat

SEQ ID NO: 50 - DNA, 19 bp, Artificial Sequence
  Artificially synthesized oligonucleotide primer
cgttgtacggcgtccagaa

SEQ ID NO: 51 - DNA, 23 bp, Artificial Sequence
  Artificially synthesized oligonucleotide primer
gaaggagggagcagtgtgtacat

SEQ ID NO: 52 - DNA, 20 bp, Artificial Sequence
  Artificially synthesized oligonucleotide primer
cccccacatagggatcatga

SEQ ID NO: 53 - DNA, 18 bp, Artificial Sequence
  Artificially synthesized oligonucleotide primer
actcctacgggaggcagc

SEQ ID NO: 54 - DNA, 23 bp, Artificial Sequence
  Artificially synthesized oligonucleotide primer
gcttcttagtcaggtaccgtcat

SEQ ID NO: 55 - DNA, 21 bp, Artificial Sequence
  Artificially synthesized oligonucleotide primer
gacgctgaggcatgagagcat

SEQ ID NO: 56 - DNA, 21 bp, Artificial Sequence
  Artificially synthesized oligonucleotide primer
gacggcacggattgttattca

SEQ ID NO: 57 - DNA, 19 bp, Artificial Sequence
  Artificially synthesized oligonucleotide primer
agcagtagggaatcttcca

SEQ ID NO: 58 - DNA, 17 bp, Artificial Sequence
  Artificially synthesized oligonucleotide primer
caccgctacacatggag

SEQ ID NO: 59 - DNA, 18 bp, Artificial Sequence
  Artificially synthesized oligonucleotide primer
ccagcagccgcggtaata

SEQ ID NO: 60 - DNA, 19 bp, Artificial Sequence
  Artificially synthesized oligonucleotide primer
cgcattccgcatacttctc

SEQ ID NO: 61 - DNA, 19 bp, Artificial Sequence
  Artificially synthesized oligonucleotide primer
ggttctgagaggaggtccc

SEQ ID NO: 62 - DNA, 17 bp, Artificial Sequence
  Artificially synthesized oligonucleotide primer
ctgcctcccgtaggagt

SEQ ID NO: 63 - DNA, 21 bp, Artificial Sequence
  Artificially synthesized oligonucleotide primer
actcctacgggaggcagcagt

SEQ ID NO: 64 - DNA, 18 bp, Artificial Sequence
  Artificially synthesized oligonucleotide primer
attaccgcggctgctggc

SEQ ID NO: 65 - DNA, 20 bp, Artificial Sequence
  Artificially synthesized oligonucleotide primer
cgcggcctatcagcttgttg

SEQ ID NO: 66 - DNA, 17 bp, Artificial Sequence
  Artificially synthesized oligonucleotide primer
attaccgcggctgctgg

SEQ ID NO: 67 - DNA, 24 bp, Artificial Sequence
  Artificially synthesized oligonucleotide primer
gctgctaataccgcatgatatgtc

SEQ ID NO: 68 - DNA, 21 bp, Artificial Sequence
  Artificially synthesized oligonucleotide primer
cagacgcgagtccatctcaga

SEQ ID NO: 69 - DNA, 23 bp, Artificial Sequence
  Artificially synthesized oligonucleotide primer
catgacgttacccgcagaagaag

SEQ ID NO: 70 - DNA, 21 bp, Artificial Sequence
  Artificially synthesized oligonucleotide primer
ctctacgagactcaagcttgc

SEQ ID NO: 71 - DNA, 20 bp, Artificial Sequence
  Artificially synthesized oligonucleotide primer
actcctacgggaggcagcag

SEQ ID NO: 72 - DNA, 20 bp, Artificial Sequence
  Artificially synthesized oligonucleotide primer
  misc_feature (7)..(7): r is a or g
  misc_feature (15)..(15): y is c or t
tctacgratttcaccyctac

SEQ ID NO: 73 - DNA, 15 bp, Artificial Sequence
  Artificially synthesized oligonucleotide primer
  misc_feature (14)..(14): y is c or t
tcactgctacacgyg

SEQ ID NO: 74 - DNA, 25 bp, Artificial Sequence
  Artificially synthesized oligonucleotide primer
  misc_feature (6)..(6): s is c or g
  misc_feature (20)..(20): r is a or g
tggcgsacgggtgagtaatrtatag

SEQ ID NO: 75 - DNA, 24 bp, Artificial Sequence
  Artificially synthesized oligonucleotide primer
  misc_feature (10)..(10): r is a or g
  misc_feature (12)..(19): w is a or t
  misc_feature (21)..(21): y is c or t
ggagtttacrcwccgaaawgygtc

SEQ ID NO: 76 - DNA, 18 bp, Artificial Sequence
  Artificially synthesized oligonucleotide primer
  misc_feature (2)..(2): m is a or c
cmatgccgcgtgtgtgaa

SEQ ID NO: 77 - DNA, 21 bp, Artificial Sequence
  Artificially synthesized oligonucleotide primer
  misc_feature (16)..(16): d is a or g or t
actccccaggcggtcdactta

SEQ ID NO: 78 - DNA, 20 bp, Artificial Sequence
  Artificially synthesized oligonucleotide primer
cgggtgagtaatgcgtgacc

SEQ ID NO: 79 - DNA, 18 bp, Artificial Sequence
  Artificially synthesized oligonucleotide primer
tgataggacgcgacccca

SEQ ID NO: 80 - DNA, 16 bp, Artificial Sequence
  Artificially synthesized oligonucleotide primer
gcacaagcagtggagt

SEQ ID NO: 81 - DNA, 18 bp, Artificial Sequence
  Artificially synthesized oligonucleotide primer
cttcctccgttttgtcaa

SEQ ID NO: 82 - DNA, 21 bp, Artificial Sequence
  Artificially synthesized oligonucleotide primer
catgtggtttaattcgatgat

SEQ ID NO: 83 - DNA, 20 bp, Artificial Sequence
  Artificially synthesized oligonucleotide primer
agctgacgacaaccatgcag

SEQ ID NO: 84 - DNA, 20 bp, Artificial Sequence
  Artificially synthesized oligonucleotide primer
agagtttgatcctggctcag

SEQ ID NO: 85 - DNA, 30 bp, Artificial Sequence
  Artificially synthesized oligonucleotide primer
ccatctcatccctgcgtgtctccgactcag

SEQ ID NO: 86 - DNA, 30 bp, Artificial Sequence
  Artificially synthesized oligonucleotide primer
cctatcccctgtgtgccttggcagtctcag

(19) United States
(12) Patent Application Publication
(10) Pub. No.: US 2020/0046788 A1
(43) Pub. Date: Feb. 13, 2020

(54) COMPOSITIONS AND METHODS FOR TREATMENT OF INTESTINAL INFLAMMATION AND COLON CANCER

(71) Applicant: University of Louisville Research Foundation, Inc., Louisville, KY (US)

(72) Inventor: Huang-Ge Zhang, Louisville, KY (US)

(73) Assignee: University of Louisville Research Foundation, Inc., Louisville, KY (US)

(21) Appl. No.: 16/462,715

(22) PCT Filed: Nov. 22, 2017
(86) PCT No.: PCT/US17/62970

§ 371 Date: May 21, 2019

Related U.S. Application Data
(60) Provisional application No. 62/425,320, filed on Nov. 22, 2016.

(51) Int. Cl. A61K 36/31; A61K 31/26; A61K 9/00; A61K 9/51; A61P 1/00

(52) U.S. Cl. CPC A61K 36/31; A61K 31/26; A61P 1/00; A61K 9/5176; A61K 9/0053

(57) ABSTRACT

Methods for treating intestinal inflammation and/or colon cancer include administering an amount of a broccoli-derived nanoparticle to a subject in need thereof. Pharmaceutical compositions comprising broccoli-derived nanoparticles are also provided. Further provided are methods for screening for a compound useful for treating a colon cancer that include the steps of contacting a provided intestinal epithelial cell with a test compound and measuring an amount of expression of COP9 signalsome subunit 8 (CSN8) in the intestinal epithelial cell to thereby identify the test compound as useful for treating colon cancer.

### RELATED APPLICATIONS

This application claims priority from U.S. Provisional Application Ser. No. 62/425,320, filed Nov. 22, 2016, the entire disclosure of which is incorporated herein by this reference.

### GOVERNMENT INTEREST

This invention was made with government support under grant numbers UH3TR000875 and R01AT008617 awarded by the National Institutes of Health. The government has certain rights in the invention.

### TECHNICAL FIELD

The presently-disclosed subject matter generally relates to compositions and methods for the treatment of intestinal inflammation and colon cancer. In particular, certain embodiments of the presently-disclosed subject matter relate to compositions and methods for treatment of intestinal inflammation and colon cancer that make use of an effective amount of broccoli-derived nanoparticles.

### BACKGROUND

The intestinal immune system is exposed daily to nanoparticles from food including edible plants. In the healthy human intestine, a constant homeostasis is maintained by an appropriate regulation of foreign antigens including the food-derived antigen load and the immune response generated against it. Failure of this balance may result in various pathological conditions.

Recently, adenosine monophosphate-activated protein kinase (AMPK) has emerged as an important enzyme and enzymatic pathway involved in the regulation of immune homeostatic networks. AMPK is expressed in several immune cell types including macrophages, lymphocytes, neutrophils and dendritic cells (DCs), and governs a broad array of immune cell functions, which include cytokine production, chemotaxis, cytotoxicity, apoptosis and proliferation. Similar to AMPK, the COP9 signalsome subunit 8 protein (CSN8), which is encoded by the CSN8 gene and is one of the eight subunits of COP9 signalosome, is a highly conserved protein complex that functions as an important regulator in multiple signaling pathways involved in inflammation, protein degradation, transcriptional activation, and signal transduction. Multiple lines of evidence have suggested that the COP9 signalosome (CSN) plays a role in the regulation of multiple cancers and could be a target for therapeutic intervention.

To date, however, the detailed biological functions of AMPK and CSN8 in regulating intestinal inflammation and tumorigenic processes during colon cancer development have not been well established, and thus, the identification and use of therapeutic agents affecting those therapeutic targets has yet to be fully realized.

### SUMMARY

The presently-disclosed subject matter meets some or all of the above-identified needs, as will become evident to those of ordinary skill in the art after a study of information provided in this document.

This summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

The presently-disclosed subject matter includes compositions and methods for the treatment of intestinal inflammation and colon cancer. In particular, the presently-disclosed subject matter includes compositions and methods for treatment of intestinal inflammation and colon cancer that make use of an effective amount of broccoli-derived nanoparticles

In some embodiments of the presently-disclosed subject matter, methods of treating intestinal inflammation are provided. In some embodiments, a method of treating intestinal inflammation is provided that comprises administering to a subject in need thereof an effective amount of a broccoli-derived nanoparticle. In some embodiments, the intestinal inflammation is colitis. In some embodiments, the broccoli-derived nanoparticle is administered orally. In some embodiments, the broccoli-derived nanoparticle includes an effective amount of sulforaphane.

With further respect to the administration of the broccoli-derived nanoparticle, in some embodiments, administering the broccoli-derived nanoparticle increases an amount of adenosine monophosphate-activated protein kinase (AMPK) signaling in the subject. In some embodiments, administering the broccoli-derived nanoparticle reduces an amount of an inflammatory cytokine in the subject, such as, in certain embodiments, interferon γ, tumor necrosis factor-α, and/or interleukin 17A. In some embodiments, administering the broccoli-derived nanoparticle reduces an amount of dendritic cell activation and/or increases an amount of dendritic cell tolerance in the subject.

Further provided, in some embodiments of the presently-disclosed subject matter are methods of treating a colon cancer. In some embodiments, a method of treating a colon cancer is provided that includes administering to a subject in need thereof an effective amount of a broccoli-derived nanoparticle. In some embodiments, administering the broccoli-derived nanoparticle decreases an amount of expression of COP9 signalsome subunit 8 (CSN8). In some embodiments, administering the broccoli-derived nanoparticle reduces an amount of polyamine metabolism in an intestinal epithelial cell of the subject. In some embodiments, administering the broccoli-derived nanoparticle reduces an amount of rectal prolapse in the subject.

In some embodiments of the presently-disclosed methods for treating a cancer, administering the broccoli-derived nanoparticle reduces an amount of inflammation in the colon of the subject. For instance, in some embodiments, administering the broccoli-derived nanoparticle reduces an amount of an inflammatory cytokine and/or reduces an amount of an inflammatory chemokine in the subject. In some embodiments, the inflammatory cytokine is selected from interleukin 22, tumor necrosis factor-α, and interleukin 17A, and the inflammatory chemokine is selected from CCL20, CXCL1, and CCL25.

In some embodiments of the therapeutic methods described herein, administering the broccoli-derived nanoparticle restores the gut microbiota in the subject. For example, in some embodiments, administering the broccoli-derived nanoparticle increases an amount of Bacteroidetes bacteria, reduces an amount of Actinobacteria bacteria, and/or reduces an amount of Proteobacteria bacteria present in the colon of the subject. In some embodiments, administering the broccoli-derived nanoparticle increases an amount of an antimicrobial peptide in an intestinal epithelial cell of the subject.

Still further provided, in some embodiments of the presently-disclosed subject matter are pharmaceutical compositions comprising an effective amount of the broccoli-derived nanoparticles described herein. In some embodiments, a pharmaceutical composition is provided that comprises a broccoli-derived nanoparticle and a pharmaceutically-acceptable vehicle, carrier, or excipient. In some embodiments, the broccoli-derived nanoparticles included in the pharmaceutical compositions include an effective amount of sulforaphane.

Even further provided, in some embodiments of the presently-disclosed subject matter are methods for screening for compounds useful for treating a colon cancer. In some embodiments, a method for screening for a compound useful for treating a colon cancer is provided that comprises the steps of: providing an intestinal epithelial cell; contacting the intestinal epithelial cell with a test compound; measuring an amount of expression of COP9 signalsome subunit 8 (CSN8) in the intestinal epithelial cell; and identifying the test compound as useful for treating colon cancer if the amount of CSN8 expression in the intestinal epithelial cell is decreased relative to a control amount of CSN8 expression.

Further advantages of the presently-disclosed subject matter will become evident to those of ordinary skill in the art after a study of the description, Figures, and non-limiting Examples in this document.

### REFERENCE TO SEQUENCE LISTING

The Sequence Listing associated with the instant disclosure has been submitted as a 20 KB file in ASCII format, created on Oct. 30, 2019 with the file name 1577-49 PCT-US_ST25.txt. The Sequence Listing is hereby incorporated by reference in its entirety into the instant disclosure.

### BRIEF DESCRIPTION OF THE DRAWINGS

FIGS. 1A-1G includes graphs and images showing that broccoli-derived nanoparticle (BDN) administration protects against (FIGS. 1A-1E) intestinal inflammation and from dextran sulfate sodium (DSS)-induced colitis where C57BL/6 mice were given BDNs orally (250 μg/mouse in 200 μL PBS) before (every day for 10 d) and after (every 2 days for 12 d) administration of water containing DSS (2.5% DSS), and also showing that BDN administration protects against (FIGS. 1F-1H) T cell mediated colitis where C57BL/6 mice were given BDNs orally (250 μg/mouse in PBS) twice every week after adoptive transfer of naïve CD4⁺ T cells isolated from C57BL/6 mice (0.5×10⁶, injected i.p.), including graphs and images showing: (FIG. 1A) weight loss in animals following the induction of colitis measured as a reduction from initial weight until day of sacrifice; (FIG. 1B) representative histology staining of colon and histologic score, where data are mean±SEM. (n=7). **P<0.01 (Student's t-test); (FIG. 1C) alcian blue staining of colon; (FIG. 1D) colon length, where data are mean±SEM. (n=7), *P<0.05, (Student's t-test); (FIG. 1E) ratio of colon weight to length; (FIG. 1F) colon thickness; (FIG. 1G) histologic scoring as performed on colon sections, where data are mean±SEM. (n=7), *P<0.05, (Student's t-test); and (FIG. 1H) representative fluorescence-activated cell sorting (FACS) plots of intracellular staining of indicated cytokines in CD3⁺CD4⁺ T cells in colonic lamina propria lymphocytes (LPL), mesenteric lymph nodes (MLN), and spleen.

FIGS. 2A-2G includes graphs showing that BDNs block the differentiation of the inflammatory Gr-1⁺ monocytes into CD11b⁺ DCs during experimental colitis including graphs showing: the frequency (FIGS. 2A and 2C) and cell number (FIGS. 2B and 2D) of CD11b⁺ DCs isolated from colonic lamina propria and MLN in DSS-induced colitis (FIGS. 2A and 2B) or naïve CD4 T cell-mediated colitis (C and D); (FIG. 2E) the phenotypic characterization of CD11b⁺ DCs from colon in CD4 T cell-mediated colitis; (FIG. 2F) real-time polymerase chain reaction (PCR) for the expression of genes in CD11b⁺ DCs sorted from pooled colonic LPL in CD4 T cell-mediated colitis, where data are mean±SEM. (n=5), *P<0.05, **P<0.01 (Student's t-test); (FIG. 2G) 3×10⁶ sorted Gr1⁺CD11b⁺ckit⁻CD11c⁻CD115⁺ monocytes from BM of naïve B6 CD45.1 mice that were injected i.v. into colitic B6 CD45.2 Rag1^{-/-} mice receiving PBS or BDNs, and where 72 hr later the colon and MLN were harvested, cells were gated on the CD45.1 population, and CD11b⁺CD11c⁺MHCII⁺ cells were examined, and where staining of CD11b⁺ DCs gated on CD45.1⁺ cells from colitic mice that received Gr1⁺CD115⁺ monocytes was performed.

FIGS. 3A-3E include graphs and images showing that BDNs inhibit anti-CD40 antibody-induced colitis, where Rag1^{-/-} mice were given BDNs orally (250 μg/mouse in PBS) every day for 10 d prior to injection with anti-CD40 (200 μg) or with rat IgG2a control, including graphs and images showing: (FIG. 3A) weight loss as a percentage of the initial weight; (FIG. 3B) representative immunohistochemistry colon sections and histologic score from Rag^{-/-} mice (n=5) at day 7 after injection with anti-CD40; (FIG. 3C) disease activity index (DAI) score; (FIG. 3D) the frequency of CD11b⁺ DCs isolated from colon and MLN in anti-CD40-induced colitis; (FIG. 3E) colon samples that were stained with antibodies directed against F4/80 or CD11c, and counterstained with hematoxylin, where graphic representation of the number of macrophages and dendritic cells (DCs) was per 1-mm² high power field in sections depicted in the left panel.

FIGS. 4A-4G includes graphs and images showing BDN derived lipid endows CD11b⁺ DCs with tolerogenic potential, where (FIGS. 1A-1B) DMSO or BDN-lipid-pretreated bone marrow-derived dendritic cells (BMDCs) were stimulated with LPS (100 ng/ml) for 24 h, where (FIG. 4C-4D) carboxyfluorescein succinimidyl ester (CFSE) labeled OT-II T cells were co-cultured with lamina propria DCs and OVA peptide (5 μg/ml) in the presence of DMSO or BDN-lipid, or where (FIGS. 4E-4G) DCs were pulsed with DMSO or BDN-lipid for 24 hr, washed, and incubated with splenic CD4⁺ T cells in the presence of anti-CD3 (5 μg/ml) and anti-CD28 (5 μg/ml) for four days, including graphs and images showing: (FIG. 4A) FACS analysis of co-stimulatory molecules in BMDCs; (FIG. 4B) ELISA analysis of cytokines; (FIG. 4C) proliferation of CFSE-labeled CD4⁺ T cells; (FIG. 4D) the level of IFN-γ and TNF-α (D) in the supernatant of co-cultured cells, where data represent means±SEM. (n=5), **p<0.01, (Student's t-test); (FIG. 4E) ELISA of cytokine production in co-culture supernatants; (FIG. 4F) RT-PCR analysis of the expression of genes in CD4⁺ T cells; and (FIG. 4G) FACS analysis of T cell activation molecules determined after 4 days in culture, where data represent means±SEM (n=5), *p<0.05, **p<0.01 (Student's t-test).

FIGS. 5A-5B include graphs and images showing that BDN lipid mediated activation of DC AMPK plays a role in preventing the induction of inflammatory DC cytokines and protection of mice against mouse colitis, where (FIGS. 5A-5C) mice receiving PBS or BDNs were exposed to PBS or 2.5% DSS, where (FIG. 5A) colonic lysates were prepared and analyzed for expression and phosphorylation of the indicated proteins by western blotting, where (FIG. 5B) immunohistochemistry of colon was analyzed by anti-pS6^{ser235/236} or P70S6K antibody, where (FIG. 5C) DMSO or BDN-lipid-treated BMDCs from B6 mice were stimulated by LPS for the indicated time and cell lysates were analyzed by immunoblotting with the indicated antibodies, where (FIG. 5D) DMSO or BDN-lipid-treated BMDCs from WT or AMPKα1^{-/-} mice were stimulated by LPS and the level of IL-12 and TNF-α in the supernatant of BMDCs was determined and (FIG. 5E) an ELISA analysis was conducted of DC-T cell co-cultures similar to FIG. 4E, but also including AMPKα1^{-/-} DCs (data in FIGS. 5D and 5E) represent means±SEM (n=5), **p<0.01 (Student's t-test), where (FIGS. 5F-5I) wild-type or AMPKα1^{-/-} BMDCs were treated with CBA (20 μg/ml) in the presence of DMSO or BDN-lipid for 24-48 hr. 2×10⁶ cells were injected i.v. into each recipient (C57BL/6) mouse at day −1 and day+3 of 2.5% DSS administration, PBS as a control for BMDCs transfer, where (FIG. 5F) body weight was measured at the indicated time points after transfer of BMDCs, where (FIG. 5G) histologic scores from DC recipient animals with DSS-induced colitis were calculated, where (FIG. 5H) colon length from colitic mice, was measured, and where (FIG. 5I) cytokine analysis by qRT-PCR of RNA recovered from whole colon was conducted (data are mean±SEM. (n=5), *P<0.05, **P<0.01 (Student's t-test)).

FIGS. 6A-6G include graphs and images showing that sulforaphane (SFN) induces regulatory DCs via AMPK-mTOR signaling, including graphs and images showing: (FIGS. 6A-6B) mouse bone marrow-derived DCs differentiated with GM-CSF and IL-4 in the presence DMSO or SFN (10 μM) and activated with PBS (FIG. 6A) or LPS for 18 h (FIG. 6B) with (FIG. 6A) FACS analysis of surface markers on BMDCs and (FIG. 6B) ELISA analysis of cytokines in the supernatant determined, where data are mean±SEM. (n=5), **P<0.01 (Student's t-test); (FIGS. 6C-6D) DMSO or SFN-derived BMDCs with/without LPS stimulation incubated with splenic CD4⁺ T cells (DC/T cell ratio, 1:5) in the presence of anti-CD3 (5 μg/ml) with (FIG. 6C). ELISA analysis of IFN-γ production of the supernatant and (FIG. 6D) FACS analysis of surface markers on CD4⁺ T cells determined, where data are mean±SEM. (n=5), **P<0.01 (Student's t-test); (FIG. 6E) human DCs that were derived with DMSO or SFN (10 μM) in the presence of GM-CSF and IL-4 and activated with LPS followed by supernatants collected after 18 h and analyzed for IL-23 and IL-10 secretion by ELISA; (FIG. 6F) ELISA of IFN-γ production of the supernatants from allogeneic CD4⁺ T cells cultured for 5 d with human DCs derived from DMSO or SFN (DC/T cell ratio, 1:5), where data are mean±SEM. (n=3), **P<0.01 (Student's t-test); and (FIG. 6G) SFN or DMSO pretreated human DCs stimulated with 100 ng/ml LPS for the time points indicated with cell lysates analyzed for the phosphorylation of the indicated proteins by western blotting.

FIGS. 7A-7F include graphs and images showing that BDN SFN protects colitis by inducing regulatory DCs, including graphs and images showing: (FIG. 7A) the lipids extracted from BDN-derived liposome-like nanoparticles (LN) or LN with SFN knock-out (LN-SFN^{-/-} or knock-in (LN-SFN^{+/+}) and a standard SFN were separated on a thin-layer chromatography plate and developed, where a representative image was scanned using an Odyssey Scanner; (FIGS. 7B-7D) BMDCs that were treated with CBA (20 μg/ml) for 24-48 hr in the presence of DMSO, LN-SFN^{-/-} or LN-SFN^{+/+} and were injected i.v. (2×10⁶) into each recipient (C57B1/6) mouse at day −1 and day+3 of 2.5% DSS administration, where (FIG. 7B) body weight was measured at the indicated time points after transfer of BMDCs, where (FIG. 7C) histologic scores were calculated from DC recipient animals with DSS-induced colitis, and where (FIG. 7D) colon length from colitic mice was measured; (FIG. 7E) cytokine analysis by qRT-PCR of RNA recovered from colonic LPL1; and (FIG. 7F) the cell number of CD11b⁺ DCs isolated from colon and MLN in DSS-induced colitis after adoptive transfer, where data are mean±SEM. (n=5), *P<0.05, **P<0.01 (Student's t-test).

FIGS. 8A-8B include graphs and images showing the characterization of broccoli-derived nanoparticles (BDNs), including: (FIG. 8A) graphs showing the size and surface charge of BDNs measured using a Zetasizer; and (FIG. 8B) images showing BDNs examined under lower (upper panel) or higher magnification (bottom panel) by electron microscopy (EM).

FIGS. 9A-9B include graphs and images showing BDN treatment prevents the induction of proinflammatory cytokines in a mouse colitis model where C57BL/6 mice were given BDNs orally (250 μg/mouse in PBS) before (every day for 10 d) and after (every 2 days for 12 d) administration of non-treated drinking water (H₂O) or water containing DSS (2.5% DSS), including graphs and images showing: (FIG. 9A) RT-PCR analysis of the expression of genes encoding pro- or anti-inflammatory cytokines in the colon from PBS and BDN-treated mice with colitis, and (FIG. 9B) representative FACS plots and cumulative data of intracellular staining of indicated cytokines in CD3⁺CD4⁺ T cells from PBS and BDN-treated mice with colitis, where data are mean±SEM. (n=5), *P<0.05, **P<0.01 (Student's t-test).

FIGS. 10A-10C include graphs and images showing that BDN treatment inhibits the induction of inflammatory CD4 T cells in MLNs where Rag1-deficient mice were given BDNs orally (250 μg/mouse in PBS), twice every week after adoptive transfer of naïve CD4⁺ T cells (0.5×10⁶, injected i.p.), including graphs and images showing: (FIG. 10A) the number of CD4⁺ T cells in colonic lamina propria, MLN and spleen; (FIG. 10B) representative FACS plots of intracellular staining of TNF-α in CD3⁺CD4⁺ T cells from colonic lamina propria, MLN, and spleen; and (FIG. 9C) RT-PCR analysis of the expression of genes encoding pro- or anti-inflammatory cytokines in the colon, where data are mean±SEM. (n=5), *P<0.05, **P<0.01 (Student's t-test).

FIG. 11 includes images taken using confocal microscopy and showing that BDNs were up taken by colon and MLN DCs, where mice were gavaged with PKH26 labeled BDNs (red), frozen sections were prepared and stained with CD11c (green) for DCs.

FIGS. 12A-12B include graphs showing that BDNs suppress the expression of chemokines and chemokine receptors in T cell mediated colitis and including graphs showing RT-PCR analysis of the expression of genes encoding chemokines (FIG. 12A) and chemokine receptors (FIG. 12B) in colon, where data are mean±SEM. (n=5), **P<0.01 (Student's t-test).

FIGS. 13A-13B includes graphs showing the effects of colitis induced by anti-CD40 antibody in Rag1^{-/-} mice, including a graph showing (FIG. 13A) RT-PCR analysis of the expression of genes encoding pro-inflammatory cytokines in the colon from mice with anti-CD40 antibody-induced colitis, and a graph showing (FIG. 13B) cytokine levels in the colon that were collected at day 7 after colitis induction, where data are mean±SEM. (n=5), *P<0.05, **P<0.01 (Student's t-test)

FIG. 14 is a graphs showing FACS analysis of lipid level in colonic CD11b⁺ DCs using BODIPY 493/503 in colitic B6 CD45.2 Rag1^{-/-} mice receiving PBS or BDNs.

FIGS. 15A-15E include images and graphs showing BDN administration protects mice from DSS-induced colitis in an AMPK-dependent manner, where WT or AMPKα1^{-/-} mice were given BDNs orally (250 μg/mouse in PBS) before (every day for 10 d) and after (every 2 days for 12 d) administration of drinking water (H₂O) or water containing DSS (2.5% DSS), including graphs and images showing: (FIG. 15A) weight loss in animals following the induction of colitis measured as reduction from initial weight until day of sacrifice; (FIG. 15B) representative histology staining of colon; (FIG. 15C) colon length, where data are mean±SEM. (n=5), *P<0.05, (Student's t-test); and (FIGS. 15D-15E) RT-PCR analysis of the expression of genes encoding pro- or anti-inflammatory cytokines (FIG. 15D) and chemokines (FIG. 15E) in colon, where data are mean±SEM. (n=5), **P<0.01 (Student's t-test).

FIGS. 16A-16C includes graphs showing that most sulforaphane is associated with BDNs and microparticles isolated from broccoli, including graphs showing: (FIG. 16A) HPLC analysis of sulforaphane; and (FIGS. 16B-C) concentration of sulforaphane in lipid extracts from nanoparticles and microparticles of broccoli (FIG. 16B) or broccoli liquid extracts with/without depletion of BDN (FIG. 16C).

FIG. 17 is a schematic diagram showing BDN mediated induction of tolerogenic AMPK⁺ DC, where during the initial treatment phase, BDNs are taken up by intestinal DCs, induce the tolerogenic DCs by activation of AMPK, and where the regulatory DCs suppress the secretion of IFN-γ and IL-17A and induce the IL-10 from activated CD4⁺ T cells.

FIGS. 18A-18F include graphs and images showing that deletion of CSN8 in intestinal epithelial cells (IEC) leads to the absence of the Paneth cell lineage of distal ileum crypts, including graphs and images showing: (FIG. 18A) representative haematoxylin and eosin (H&E) stainings of ileum, with paneth cells with typical eosinophilic granules (black arrows) on H&E-stained sections at the base of crypts in CSN8^{fl/fl}, but not CSN8^{ΔIEC}, epithelium (Scale bar, 50 μm); (FIG. 18B) granule proteins lysozyme examined by immunofluorescence (Red) and counted in the ileum of CSN8^{fl/fl} and CSN8^{ΔIEC} mice (scale bar, 100 μm); (FIG. 18C) TEM of crypts of CSN8^{fl/fl} and CSN8^{ΔIEC} mice, where the base of the crypt in CSN8^{ΔIEC} mice is occupied by poorly differentiated columnar epithelial cells with lack of secretory granules, rudimentary electron-dense granules (black arrows), granules in lumen (blue arrows) and a contracted ER (white arrows); (FIG. 18D) goblet cells stained by Alcian blue and counted in CSN8^{fl/fl} and CSN8^{ΔIEC} epithelia of duodenum (D) and jejunum (J) (scale bar, 100 μm); (FIG. 18E) the marker for enteroendocrine cells, chromogranin, detected by immunofluorescence and counted in ileum (I) of CSN8^{fl/fl} and CSN8^{ΔIEC} mice (scale bar, 100 μm); and (FIG. 18F) RNA sequencing-based measurements of transcripts comprising antimicrobial peptides (AMPs)-related genes in crypts isolated from CSN8^{fl/fl} and CSN8^{ΔIEC} mice, where data are mean±SEM (n=7), *p<0.05, **p<0.01 (Student's t test).

FIGS. 19A-19G include images and graphs showing CSN8 deficiency increases susceptibility to DSS-induced colitis, including images and graphs showing: (FIG. 19A) RT-PCR analysis of differential abundance of selected bacterial taxa in stool samples of ileum and cecum from CSN8^{fl/fl} and CSN8^{ΔIEC} mice; (FIG. 19B) 16S-rRNA RT-PCR analysis of the abundance of luminal and mucosal bacteria in ileum and cecum from CSN8^{fl/fl} and CSN8^{ΔIEC} mice; (FIG. 19C) scanning electron microscopy of ileum from CSN8^{fl/fl} and CSN8^{ΔIEC} mice; and (FIG. 19D-19G) mice treated by 2% DSS in drinking water for 7-12 days with (FIG. 19D) changes in body weight presented as % of initial weight, (FIG. 19E) survival curves of CSN8^{fl/fl} and CSN8^{ΔIEC} mice during DSS colitis where difference in survival was determined with Kaplan-Meier analysis (n=20), (FIG. 19F) representative H&E stained sections of colon and cecum from CSN8^{fl/fl} and CSN8^{ΔIEC} mice after DSS colitis (day 9, scale bar, 100 μm), and (FIG. 19G) histopathological scores sections of distal colon and cecum and colon length were analyzed in CSN8^{fl/fl} and CSN8^{ΔIEC} mice after DSS colitis (day 9), where data are mean±SEM (n=20), *p<0.05, **p<0.01 (Student's t test).

FIGS. 20A-20K include images and graphs showing CSN8 deficiency in IEC strongly attenuates the initiation and progression of intestinal tumors, including images and graphs showing: (FIG. 20A) representative gross intestinal morphology observed in the small intestine of APC^{Min/+}CSN8^{fl/fl} and APC^{Min/+}CSN8^{ΔIEC} mice. Count of small and large polyp per mouse of APC^{Min/+}CSN8^{fl/fl} and APC^{Min/+}CSN8^{ΔIEC} mice (5.5 months of age, n=15); (FIG. 20B) H&E-stained sections of ileums and colons from APC^{Min/+}CSN8^{fl/fl} and APC^{Min/+}CSN8^{ΔIEC} mice (5.5 months of age), showing tumor (T) and representative inflammation (I), scale bar, 100 μm; (FIG. 20C) histopathological inflammation score of from distal ileum and colon tissue sections and percentage of rectal prolapse observed in APC^{Min/+}CSN8^{fl/fl} and APC^{Min/+}CSN8^{Min/+}CSN8^{ΔIEC}, data are mean±SEM, n=15, *P<0.05, **P<0.01. (Student's t-test); (FIG. 20D) representative gross intestinal morphology observed in the colon of CSN8^{fl/fl} and CSN8^{ΔIEC} mice after (azoxymethane) AOM/DSS administration (day 93); (FIG. 20E) count of small, medium, and large tumors per mouse of CSN8^{fl/fl} and CSN8^{ΔIEC} mice after AOM/DSS colitis (day 93); (FIG. 20F) average of tumor sizes in CSN8^{fl/fl} and CSN8^{ΔIEC} after treatment with AOM/DSS; (FIG. 20G) H&E-stained sections of colons from CSN8^{fl/fl} and CSN8^{ΔIEC} mice after AOM/DSS colitis (day 93), showing tumor and representative inflammation, scale bar, 100 μm; (FIG. 20H) histopathological inflammation score of from distal colon tissue sections in CSN8^{ΔIEC} and CSN8^{ΔIEC} mice; (FIG. 20I) percentage of survival observed in CSN8^{fl/fl} and mice during the course of AOM/DSS colitis; and (FIG. 20J) percentage of rectal prolapse observed in CSN8^{fl/fl} and CSN8^{ΔIEC} mice after AOM/DSS colitis (day 93), where data are mean±SEM, n=9, *P<0.05, **P<0.01. (Student's t-test).

FIGS. 21A-21I include images and graphs showing CSN8 deficiency modifies gut microbiota composition and bacterial diversity, including images and graphs showing: (FIG. 21A) RT-PCR analysis of the expression of genes encoding antimicrobial peptides in the ileum of APC^{Min/+}CSN8^{fl/fl} and APC^{Min/+}CSN8^{ΔIEC} mice; (FIG. 21B) principal components analysis of 16S rRNA gene-sequencing analysis of gut microbes obtained from APC^{Min/+}CSN8^{fl/fl} and APC^{Min/+}CSN8^{fl/fl} mice, where PC1 and PC2 explain 16% and 8% of variation, respectively; (FIG. 21C) rarefaction measure for diversity of the gut microbiota in fecal samples of APC^{Min/+}CSN8^{fl/fl} and APC^{Min/+}CSN8^{ΔIEC} mice; (FIG. 21D) heatmap of differentially represented bacterial species in feces between APC^{Min/+}CSN8^{fl/fl} and APC^{Min/+}CSN8^{ΔIEC} mice, where log 10-transformation was applied on the relative abundance data matrix and phyla assignment of the different bacterial species is indicated by a cap letter at the beginning of the species name. F: Firmicutes, B: Bacteroidetes, P: Proteobacteria, A: Actinobacteria; (FIG. 21E) the relative abundance of fecal bacteria phylum in the feces of APC^{Min/+}CSN8^{fl/fl} and APC^{Min/+}CSN8^{ΔIEC} mice; (FIG. 21F) LEfSe analysis applied to identify high-dimensional biomarkers that discriminate between faeces from APC^{Min/+}CSN8^{fl/fl} and APC^{Min/+}CSN8^{ΔIEC} mice; (FIG. 21G) main bacterial genera repartition in the feces of APC^{Min/+}CSN8^{fl/fl} and APC^{Min/+}CSN8^{ΔIEC} mice; (FIG. 21H) RT-PCR analysis of differential abundance of selected bacterial taxa in stool samples of APC^{Min/+}CSN8^{fl/fl} and APC^{Min/+}CSN8^{fl/fl} mice; and (FIG. 21I) culture of microbiota from colonic mucosa from APC^{Min/+}CSN8^{fl/fl} and APC^{Min/+}CSN8^{ΔIEC} mice, where data are mean±SEM. (n=15), *P<0.05, **P<0.01. (Student's t-test).

FIGS. 22A-22F include images and graphs showing that intestinal inflammation, but not tumors are transmissible by CSN8-regulated gut microbiota; including images and graphs showing: (FIG. 22A) histologic severity scores of APC^{Min/+}CSN8^{fl/fl} mice and APC^{Min/+}CSN8^{ΔIEC} after faecal transplantation, where data represents the histological score of mice analyzed at day 160; (FIG. 22B) polyps number of APC^{Min/+}CSN8^{fl/fl} mice and APC^{Min/+}CSN8^{ΔIEC} after faecal transplantation, where data represents the tumor number of mice at day 160; (FIGS. 21C-21F) APC^{Min/+}CSN8^{fl/fl} and APC^{Min/+}CSN8^{ΔIEC} that were either untreated (PBS) or treated with the antibiotic cocktail (Abx) for 3 months; (FIG. 22C) inflammatory scores in the colon of 5-month-old mice; (FIG. 22D) percentage of rectal prolapse of 5-month-old mice; (FIG. 22E) FACS analysis of CD45⁺ infiltrating immune cells in the lamina propria of ileum; and (FIG. 22F) RT-PCR analysis of the expression of genes encoding inflammatory cytokines in the ileum, where data are mean±SEM. (n=10), *P<0.05, **P<0.01, ns=no significant. (Student's t-test).

FIGS. 23A-23E include images and graphs showing CSN8 deficiency alters polyamine pools in the intestine in APC^{Min/+} mice, including images and graphs showing: (FIG. 23A) RT-PCR analysis of the expression of genes encoding enzymes of the polyamine metabolic pathway in the ileum from APC^{Min/+}CSN8^{fl/fl} and APC^{Min/+}CSN8^{ΔIEC} mice; (FIG. 23B) immunoblot analysis of ODC, SMOX and SSAT protein expression in the ileum, colon and polyps from APC^{Min/+}CSN8^{fl/fl} and APC^{Min/+}CSN8^{ΔIEC} mice; (FIG. 23C) immunohistochemical analysis of ODC and SSAT protein expression in the ileum from APC^{Min/+}CSN8^{fl/fl} and APC^{Min/+}CSN8^{ΔIEC} mice, scale bar, 100 μm; (FIG. 23D) SSAT and ODC activity levels in intestinal tissues of APC^{Min/+}CSN8^{fl/fl} and APC^{Min/+}CSN8^{ΔIEC} mice, where activities were measured in extracts of normal mucosa (Non-Tumor) and adenomatous polyps (tumor) of the ileum and colon from APC^{Min/+}CSN8^{fl/fl} and APC^{Min/+}CSN8^{fl/fl} mice; and (FIG. 23E) polyamine pools in normal intestinal tissues and polyps of APC^{Min/+}CSN8^{fl/fl} and APC^{Min/+}CSN8^{ΔIEC} mice, where targeted metabolomics, concentrations of polyamines and polyamine metabolites were determined for normal regions and polyps of ileum, and where data are mean±SEM. (n=10), *P<0.05, **P<0.01 (Student's t-test).

FIGS. 24A-24K include images and graphs showing dietary supplementation with BDNs significantly decreased intestinal and colonic polyp number and size in APC^{min/+} mice, wherein 5 weeks-age APC^{min/+} mice were administered 25 mg/kg body weight BDNs or vehicle every two days for about 2.5 months, and where mice were killed and intestinal tissues harvested at the age of 90-100 days, and including images and graphs showing: (FIG. 24A) polyp number and representative gross intestinal morphology observed in small intestine and large intestine; (FIG. 24B) polyp size in small intestine and large intestine, where data are mean±SEM. (n=10), **P<0.01; (FIG. 24C) immunofluorescence analysis of Ki-67 expression in the intestine from APC^{Min/+} mice with/without BDNs supplement, scale bar, 100 μm; (FIG. 24D) immunoblot analysis for indicated protein in lysates from the small intestine and colon in APC^{Min/+} mice with/without BDNs supplement; (FIG. 24E) immunohistochemical analysis of phospho-STAT3 protein, CSN8 and Nrf2 expression with primary antibody in the ileum from APC^{Min/+} mice with/without BDNs supplement, scale bar, 100 μm; (FIG. 24F) polyamine pools and polyamine metabolites in the polyps of APC^{Min/+} mice with/without BDNs supplement, where data are mean±SEM, (n=5), *P<0.05, **P<0.01; (FIG. 24G) RT-PCR analysis of the expression of genes encoding enzymes of the polyamine metabolic pathway in the ileum from APC^{Min/+} mice with/without BDNs supplement, where data are mean±SEM, (n=5), *P<0.05, **P<0.01; (FIG. 24H) immunoblot analysis of ODC, SMOX and SSAT protein expression in the ileum from APC^{Min/+} mice with/without BDNs supplement; (FIG. 24I) MC38 cells treated by DMSO, SFN, or BDNs-derived lipids followed by RT-PCR analysis of the expression of genes encoding Nrf2, CSN8 and enzymes of the polyamine metabolic pathway; (FIG. 24J) Immunoblot analysis of nuclear Nrf2 protein expression in the ileum from APC^{Min/+}CSN8^{fl/fl} and APC^{Min/+}CSN8^{ΔIEC} mice or MC38 cells with/without CSN8 knockdown; and (FIG. 24K) MC38 cells knocked down by CSN8, Nrf2, or both and treated by DMSO or SFN followed by RT-PCR analysis of the expression of genes encoding SSAT1, where data are mean±SEM, (n=5), *P<0.05, **P<0.01, (Student's t-test).

FIGS. 25A-25F include images and graphs showing that dietary supplementation with BDNs significantly restores the homeostasis of gut microbiota and decreases the intestinal inflammation in APC^{Min/+} mice, including images and graphs showing (FIG. 25A) 16S rRNA gene sequencing analysis at the phylum levels of cecum content after 2.5 months of BDNs treatment in APC^{min/+} mice; (FIG. 25B) 16S rDNA RT-PCR analysis of genus levels of cecum content after 2.5 months of BDNs treatment in APC^{min/+} mice; (FIG. 25C) RT-PCR analysis of the expression of genes encoding antimicrobial peptides in the ileum of APC^{Min/+} mice 2.5 months of BDNs treatment; (FIG. 25D) RT-PCR analysis of the expression of genes encoding cytokines and chemokines in the ileum of APC^{Min/+} mice after 2.5 months of BDNs treatment; (FIG. 25E) histopathological inflammation score of from colon tissue sections and percentage of rectal prolapse observed in APC^{Min/+}CSN8^{ΔIEC} mice after 4 months of BDNs treatment; and (FIG. 25F) rectal prolapse of APC^{Min/+}CSN8^{ΔIEC} mice after 4 months of BDNs treatment, where data are mean±SEM, (n=7), *P<0.05, **P<0.01, (Student's t-test).

FIGS. 26A-26C include images and graphs showing deletion of CSN8 in IEC affects the COP9 complex integrity, including images and graphs showing: (FIG. 26A) immunofluorescence analysis of CSN8 protein expression with anti-CSN8 antibody (red) and with DAPI (blue) in the duodenum (D), jejunum (J), ileum (I) and large intestine (LI) from CSN8^{fl/fl} and CSN8^{ΔIEC} mice, scale bar, 100 μm; (FIG. 26B) immunoblot analysis of CSN8 protein expression in the liver, kidney, lung and IEC isolated from ileum from CSN8^{fl/fl} and CSN8^{ΔIEC} mice; and (FIG. 26C) immunoblot analysis of the indicated proteins in lysates prepared from isolated IEC from CSN8^{fl/fl} and CSN8^{ΔIEC} mice.

FIGS. 27A-27C include images and graphs showing deletion of CSN8 in IEC affects body weight, including images and graphs showing: (FIG. 27A) body size and weight of CSN8^{fl/fl} and CSN8^{ΔIEC} mice at the age of 4 weeks; and (FIG. 27B) Length of small intestinal and (FIG. 27C) colon from CSN8^{fl/fl} and CSN8^{ΔIEC} mice, where data are mean±SEM, (n=7), *p<0.05, **p<0.01 (Student's t test).

FIGS. 28A-28E include images and graphs showing paneth cell loss in CSN8^{ΔIEC} mice, including images and graphs showing: (FIG. 28A) representative haematoxylin and eosin staining of duodenum (D) and jejunum (J), scale bar, 50 μm; (FIG. 28B) the granule proteins lysozyme examined by immunofluorescence (Red) and counted in the duodenum (D) and jejunum (J) from CSN8^{fl/fl} and CSN8^{ΔIEC} mice, scale bar, 100 μm; (FIG. 28C) TEM of crypts of CSN8^{fl/fl} and CSN8^{ΔIEC} mice, where the base of the crypt in CSN8^{ΔIEC} mice is occupied by poorly differentiated columnar epithelial cells with lack of secretory granules, rudimentary electron-dense granules (black arrows), microvilli (yellow arrows) and granules in lumen (blue arrows); (FIG. 28D) goblet cells were stained by Alcian blue stain and counted in the ileum (I) from CSN8^{fl/fl} and CSN8^{ΔIEC} mice, scale bar, 50 μm; and (FIG. 28E) the marker for enteroendocrine cells, chromogranin, as detected by immunofluorescence and counted in the duodenum (D) and jejunum (J) from CSN8^{fl/fl} and CSN8^{ΔIEC} mice, scale bar, 100 μm.

FIGS. 29A-29B include graphs showing CSN8 regulates the expression of antimicrobial peptides (AMPs) as measured by RT-PCR analysis of the expression of genes encoding AMPs in the ileum (FIG. 29A) and colon (FIG. 29B) in CSN8^{fl/fl} and CSN8^{ΔIEC} mice, where data are mean±SEM, (n=5), ***P<0.001, **P<0.01, *P<0.05 (Student's t-test).

FIGS. 30A-30C includes graphs showing CSN8 deficiency promotes DSS-induced inflammation, where WT or CSN8^{ΔIEC} mice were treated by 2% DSS in drinking water for 7-12 days, including graphs showing: (FIG. 30A) cytokine levels in the colon that were collected at day 7 after colitis induction, where data are mean±SEM. (n=5), *P<0.05, **P<0.01 (Student's t-test); (FIG. 30B) the frequency of CD11b⁺Ly6C⁺ and CD11b⁺Ly6G⁺ cells in colonic lamina propria (cLP) from WT or CSN8^{ΔIEC} mice with DSS-induced colitis; and (FIG. 30C) representative FACS plots and percentage of intracellular staining of Foxp3, IFN-γ and IL-17A in CD3⁺CD4⁺ T cells from colonic lamina propria from WT or CSN8^{ΔIEC} mice with DSS-induced colitis.

FIGS. 31A-31E include graphs and images showing CSN8^{ΔIEC} mice are highly susceptive to DSS induced colitis, where WT or CSN8^{ΔIEC} mice were treated by 2% DSS in drinking water for 7-12 days and the distal ileum was examined, including graphs and images showing: (FIG. 31A) distal ileum histology on day 9 of DSS colitis; (FIG. 31B) histopathological scores analyzed from sections of distal ileum on day 9 of DSS colitis, scale bar, 100 μm; (FIG. 31C) cytokine levels in the ileum that were collected at day 7 after colitis induction; (FIG. 31D) the frequency of CD11b⁺Ly6C⁺ and CD11b⁺Ly6G⁺ cells in lamina propria in the ileum; (FIG. 31E) representative FACS plots of intracellular staining of Foxp3, IFN-γ and IL-17A in CD3⁺CD4⁺ T cells from lamina propria in the ileum, were data are mean±SEM, (n=5), *P<0.05, (Student's t-test).

FIGS. 32A-32D include graphs showing CSN8 deficiency promotes intestinal inflammation in APC^{min/+} mice, including graphs showing: (FIG. 32A) representative FACS plots of intracellular staining of Foxp3, IFN-γ and IL-17A in CD3⁺CD4⁺ T cells from the lamina propria of ileum and colon in APC^{Min/+}CSN8^{fl/fl} and APC^{Min/+}CSN8^{ΔIEC} mice; (FIG. 32B) representative FACS plots of CD11b⁺F4/80⁺ macrophages, CD11b⁺Gr-1⁺MDSC, CD11c⁺MHCII⁺ DCs from the lamina propria of ileum and colon APC^{Min/+}CSN8^{fl/fl} and APC^{Min/+}CSN8^{ΔIEC} mice; and (FIGS. 32C-32D) RT-PCR analysis of the expression of genes encoding cytokines (FIG. 32C) and chemokines (FIG. 32D) in the colon in APC^{Min/+}CSN8^{fl/fl} and APC^{Min/+}CSN8^{ΔIEC} mice, where data are mean±SEM, n=9, *P<0.05, **P<0.01 (Student's t-test).

FIGS. 33A-33C include graphs showing CSN8 deficiency promotes intestinal inflammation in AOM/DSS mice, including graphs showing: (FIG. 33A) representative FACS plots of CD11b⁺CD11c⁺ myeloid cells and intracellular staining of Foxp3, IFN-γ and IL-17A in CD3⁺CD4⁺ T cells from the lamina propria of colon in CSN8^{fl/fl} and CSN8^{ΔIEC} mice after AOM/DSS colitis (day 93); and (FIGS. 33B-33C) RT-PCR analysis of the expression of genes encoding cytokines (FIG. 33B) and chemokines (FIG. 33C) in colon of CSN8^{fl/fl} and CSN8^{ΔIEC} mice after AOM/DSS colitis (day 93), where data are mean±SEM. (n=5), *P<0.05, **P<0.01 (Student's t-test).

FIGS. 34A-34E include graphs showing loss of CSN8 in intestinal epithelial cells drives enterocytes to apoptosis, including graphs showing: (FIG. 34A) immunofluorescence analysis of Ki-67 expression in the normal mucosa (Non-Tumor) and adenomatous polyps (tumor) of the ileum from APC^{Min/+}CSN8^{fl/fl} and APC^{Min/+}CSN8^{ΔIEC} mice, scale bar, 100 μm; (FIG. 34B) CSN8^{fl/fl} and CSN8^{ΔIEC} mice administered bromodeoxyuridine (BrdU) intraperitoneally, and small intestinal sections harvested after 24 and 48 hr, where the 24 hr time point labels the pool of proliferating IECs in the crypts (mostly transit amplifying IEC), while the 48 hr time point assesses the migration along the crypt-villus axis indicating the turnover of the IEC compartment, scale bar, 100 μm; (FIG. 34C) quantification of intestinal epithelial cell migration along the crypt-villus axis; (FIG. 34D) immunofluorescence analysis of cleaved caspase 3 in the ileum from APC^{Min/+}CSN8^{fl/fl} and APC^{Min/+}CSN8^{fl/fl} mice; and (FIG. 34E) immunoblot analysis of cleaved caspase 3 in the ileum from APC^{Min/+}CSN8^{fl/fl} and APC^{Min/+}CSN8^{fl/fl} mice, where data are mean±SEM, (n=5), *P<0.05, **P<0.01 (Student's t-test).

FIGS. 35A-35F include images showing loss of CSN8 affects the cell-cycle of intestinal epithelial cells via polyamine-regulated markers, including images showing: (FIGS. 35A-35B) immunofluorescence analysis of phospho-STAT3 expression in normal mucosa (non-tumor) and adenomatous polyps (tumor) of the small intestine and colon from APC^{Min/+}CSN8^{fl/fl} and APC^{Min/+}CSN8^{ΔIEC} mice, scale bar, 100 μm; (FIG. 35C) immunoblot analysis for phospho-STAT3 and STAT3 in lysates from normal mucosa (non-tumor) of the small intestine and colon from APC^{Min/+}CSN8^{fl/fl} and APC^{Min/+}CSN8^{ΔIEC} mice; (FIG. 35D) immunoblot analysis for phospho-STAT3 and STAT3 in lysates from adenomatous polyps (tumor) of the small intestine from APC^{Min/+}CSN8^{fl/fl} and APC^{Min/+}CSN8^{ΔIEC} mice; and (FIGS. 35E-35F) immunoblot analysis for polyamine-related cell cycle targets in lysates from normal mucosa (Non-Tumor) of the small intestine and colon from APC^{Min/+}CSN8^{fl/fl} and APC^{Min/+}CSN8^{ΔIEC} mice.

FIGS. 36A-36D include images and graphs showings suppression of intestinal tumorigenesis by CSN8 can be reversed by exogenous putrescine, where 8 weeks-age APC^{Min/+}CSN8^{fl/fl} and APC^{Min/+}CSN8^{ΔIEC} mice were treated with PBS or putrescine (1% in the drinking water) for about 3 month and where mice were killed and intestinal tissues harvested on day 120, including images and graphs showing: (FIG. 36A) polyamine pools in normal intestinal tissues and polyps of APC^{Min/+}CSN8^{ΔIEC} and APC^{Min/+}CSN8^{fl/fl} mice with/without putrescine supplement; (FIG. 36B) polyp numbers in the small intestine and large intestine of APC^{Min/+}CSN8^{fl/fl} and APC^{Min/+}CSN8^{ΔIEC} mice with/without putrescine supplement; (FIG. 36C) immunofluorescence analysis of Ki-67 expression in the intestine from APC^{Min/+}CSN8^{fl/fl} and APC^{Min/+}CSN8^{ΔIEC} mice, scale bar, 100 μm; and (FIG. 36D) Immunoblot analysis for polyamine-related cell cycle targets in lysates from the small intestine from APC^{Min/+}CSN8^{fl/fl} and APC^{Min/+}CSN8^{ΔIEC} mice with/without putrescine supplement, where data are mean±SEM, (n=5), *P<0.05, **P<0.01 (Student's t-test).

FIGS. 37A-37B are graphs showing dietary supplementation with BDNs inhibit gut inflammation, where 5 weeks-age APC^{min/+} mice were administered 25 mg/kg body weight BDNs or vehicle every two days for about 2.5 months and mice were killed and intestinal tissues harvested at the age of day 90-100, including graphs showing: (FIG. 37A) representative FACS plots of intracellular staining of Foxp3, TNF-α, IL-5, IFN-γ and IL-17A in CD3⁺CD4⁺ T cells in the ileum of APC^{Min/+} mice after 2.5 months of BDNs treatment; and (FIG. 37B) The frequency of CD11b⁺F4/80⁺ and CD11b⁺Gr-1⁺ cells in the ileum of APC^{Min/+} mice after 2.5 months of BDNs treatment.

### DESCRIPTION OF EXEMPLARY EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by those of ordinary skill in the art, certain definitions are set forth to facilitate explanation of the presently-disclosed subject matter.

Unless defined otherwise, all technical and scientific terms used herein have the same meaning as is commonly understood by one of skill in the art to which the inventions) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. (1972) 11(9): 1726-1732).

Although any methods, devices, and materials similar or equivalent to those described herein can be used in the practice or testing of the presently-disclosed subject matter, representative methods, devices, and materials are described herein.

The present application can “comprise” (open ended), “consist of” (closed), or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a nanoparticle” includes a plurality of such nanoparticles, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

The presently-disclosed subject matter includes compositions and methods for the treatment of intestinal inflammation and colon cancer. In particular, certain embodiments of the presently-disclosed subject matter relate to compositions and methods for treatment of intestinal inflammation and colon cancer that make use of an effective amount of broccoli-derived nanoparticles.

The term “nanoparticles” as used herein in reference to the broccoli-derived nanoparticles of the presently disclosed subject matter, refers to nanoparticles that are in the form of small assemblies of lipid particles, are about 50 to 1000 ran in size, and are not only secreted by many types of in vitro cell cultures and in vivo cells, but are commonly found in vivo in body fluids, such as blood, urine and malignant ascites. Indeed, such nanoparticles include, but are not limited to, particles such as microvesicles, exosomes, epididimosomes, argosomes, exosome-like vesicles, microparticles, promininosomes, prostasomes, dexosomes, texosomes, dex, tex, archeo somes, and oncosomes.

Such nanoparticles can be formed by a variety of processes, including the release of apoptotic bodies, the budding of microvesicles directly from the cytoplasmic membrane of a cell, and exocytosis from multivesicular bodies. For example, exosomes are commonly formed by their secretion from the endosomal membrane compartments of cells as a consequence of the fusion of multivesicular bodies with the plasma membrane. The multivesicular bodies are formed by inward budding from the endosomal membrane and subsequent pinching off of small vesicles into the luminal space. The internal vesicles present in the multivesicular bodies are then released into the extracellular fluid as so-called exosomes.

As part of the formation and release of nanoparticles, unwanted molecules are eliminated from cells. However, cytosolic and plasma membrane proteins are also incorporated during these processes into the microvesicles, resulting in microvesicles having particle size properties, lipid bilayer functional properties, and other unique functional properties that allow the nanoparticles to potentially function as effective nanoparticle carriers of therapeutic agents. In this regard, the term “nanoparticle” is used interchangeably herein with the terms “microvesicle,” “liposome,” “exosome,” “exosome-like particle,” “nano-vector” and grammatical variations of each of the foregoing.

The phrase “derived from broccoli” or “broccoli-derived” when used in the context of a nanoparticle, refers to a nanoparticle that, by the hand of man, exists apart from its native environment and is therefore not a product of nature. In this regard, in some embodiments, the phrase “derived from broccoli” can be used interchangeably with the phrase “isolated from broccoli” to describe a nanoparticle of the presently-disclosed subject matter. For example, in some embodiments of the presently-disclosed subject matter, nanoparticles derived from broccoli can be produced by first grinding whole broccoli plants in a blender at high speeds and for a sufficient period of time to produce a juice of the broccoli. The broccoli juice can then be subsequently and sequentially centrifuged at increasing speeds and for increasing periods of time (e.g., 1000 g for 10 min, 3000 g for 20 min, and 10,000 g for 40 min) to produce a microparticle pellet and supernatant. That resulting supernatant can then be further centrifuged at higher speeds and an additional period of time (e.g., 100,000 g for 90 min) and subsequently exposed to a sucrose purification for isolation of nanoparticles. For further information and guidance regarding the production of plant-derived nanoparticles, see, e.g., Mu, et al., Mol. Nutr. Food Res. 58, 1561-1573, which is incorporated herein by reference in its entirety. After isolation, the nanoparticles can then be collected, washed, and dissolved in a suitable solution for therapeutic use. In some embodiments, by making use of the methods described herein, the broccoli-derived nanoparticles include an effective amount and/or are enriched in sulforaphane.

In some embodiments of the presently-disclosed subject matter, a pharmaceutical composition is thus provided that comprises a broccoli-derived nanoparticle disclosed herein and a pharmaceutical vehicle, carrier, or excipient. In some embodiments, the pharmaceutical composition is pharmaceutically-acceptable in humans. Also, as described further below, in some embodiments, the pharmaceutical composition can be formulated as a therapeutic composition for delivery to a subject.

A pharmaceutical composition as described herein preferably comprises a composition that includes pharmaceutical carrier such as aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. The pharmaceutical compositions used can take such forms as suspensions, solutions or emulsions in oily or aqueous vehicles, and can contain formulatory agents such as suspending, stabilizing and/or dispersing agents. Additionally, the formulations can be presented in unit-dose or multi-dose containers, for example sealed ampoules and vials, and can be stored in a frozen or freeze-dried or room temperature (lyophilized) condition requiring only the addition of sterile liquid carrier immediately prior to use.

In some embodiments, solid formulations of the compositions for oral administration can contain suitable carriers or excipients, such as corn starch, gelatin, lactose, acacia, sucrose, microcrystalline cellulose, kaolin, mannitol, dicalcium phosphate, calcium carbonate, sodium chloride, or alginic acid. Disintegrators that can be used include, but are not limited to, microcrystalline cellulose, corn starch, sodium starch glycolate, and alginic acid. Tablet binders that can be used include acacia, methyl cellulose, sodium carboxymethylcellulose, polyvinylpyrrolidone, hydroxypropyl methylcellulose, sucrose, starch, and ethylcellulose. Lubricants that can be used include magnesium stearates, stearic acid, silicone fluid, talc, waxes, oils, and colloidal silica. Further, the solid formulations can be uncoated or they can be coated by known techniques to delay disintegration and absorption in the gastrointestinal tract and thereby provide a sustained/extended action over a longer period of time. For example, glyceryl monostearate or glyceryl distearate can be employed to provide a sustained-/extended-release formulation. Numerous techniques for formulating sustained release preparations are known to those of ordinary skill in the art and can be used in accordance with the present invention, including the techniques described in the following references: U.S. Pat. Nos. 4,891,223; 6,004,582; 5,397,574; 5,419,917; 5,458,005; 5,458,887; 5,458,888; 5,472,708; 6,106,862; 6,103,263; 6,099,862; 6,099,859; 6,096,340; 6,077,541; 5,916,595; 5,837,379; 5,834,023; 5,885,616; 5,456,921; 5,603,956; 5,512,297; 5,399,362; 5,399,359; 5,399,358; 5,725,883; 5,773,025; 6,110,498; 5,952,004; 5,912,013; 5,897,876; 5,824,638; 5,464,633; 5,422,123; and 4,839,177; and WO 98/47491, each of which is incorporated herein by this reference.

Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically-acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of capsules, tablets or lozenges formulated in conventional manner.

Various liquid and powder formulations can also be prepared by conventional methods for inhalation into the lungs of the subject to be treated or for intranasal administration into the nose and sinus cavities of a subject to be treated. For example, the compositions can be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. Capsules and cartridges of, for example, gelatin for use in an inhaler or insufflator may be formulated containing a powder mix of the desired compound and a suitable powder base such as lactose or starch.

The compositions can also be formulated as a preparation for implantation or injection. Thus, for example, the compositions can be formulated with suitable polymeric or hydrophobic materials (e.g., as an emulsion in an acceptable oil) or ion exchange resins, or as sparingly soluble derivatives (e.g., as a sparingly soluble salt).

Injectable formulations of the compositions can contain various carriers such as vegetable oils, dimethylacetamide, dimethylformamide, ethyl lactate, ethyl carbonate, isopropyl myristate, ethanol, polyols (glycerol, propylene glycol, liquid polyethylene glycol), and the like. For intravenous injections, water soluble versions of the compositions can be administered by the drip method, whereby a formulation including a pharmaceutical composition of the presently-disclosed subject matter and a physiologically-acceptable excipient is infused. Physiologically-acceptable excipients can include, for example, 5% dextrose, 0.9% saline, Ringer's solution or other suitable excipients. Intramuscular preparations, e.g., a sterile formulation of a suitable soluble salt form of the compounds, can be dissolved and administered in a pharmaceutical excipient such as Water-for-Injection, 0.9% saline, or 5% glucose solution. A suitable insoluble form of the composition can be prepared and administered as a suspension in an aqueous base or a pharmaceutically-acceptable oil base, such as an ester of a long chain fatty acid, (e.g., ethyl oleate).

In addition to the formulations described above, the broccoli-derived nanoparticle compositions of the presently-disclosed subject matter can also be formulated as rectal compositions, such as suppositories or retention enemas, e.g., containing conventional suppository bases such as cocoa butter or other glycerides. Further, the broccoli-derived nanoparticle compositions can also be formulated as a depot preparation by combining the compositions with suitable polymeric or hydrophobic materials (for example as an emulsion in an acceptable oil) or ion exchange resins, or as sparingly soluble derivatives, for example, as a sparingly soluble salt capable of use in a therapeutic application.

Turning now to the therapeutic uses of the broccoli-derived nanoparticles of the presently-disclosed subject matter, in some embodiments, methods for treating intestinal inflammation or a colon cancer are provided. In some embodiments, a method for treating intestinal inflammation is provided that comprises administering to a subject in need thereof an effective amount of a broccoli-derived nanoparticle. In some embodiments of the therapeutic treatment, the methods further include a step of selecting a broccoli-derived nanoparticle of the presently-disclosed subject matter prior to administering the nanoparticle to the subject.

As used herein, the terms “treatment” or “treating” relate to any treatment of a condition of interest (e.g., an intestinal inflammation or a cancer), including but not limited to prophylactic treatment and therapeutic treatment. As such, the terms “treatment” or “treating” include, but are not limited to: preventing a condition of interest or the development of a condition of interest; inhibiting the progression of a condition of interest; arresting or preventing the further development of a condition of interest; reducing the severity of a condition of interest; ameliorating or relieving symptoms associated with a condition of interest; and causing a regression of a condition of interest or one or more of the symptoms associated with a condition of interest.

As used herein, the term “intestinal inflammation” is used to refer to inflammation, which is generally characterized by increased blood flow, edema, activation of immune cells (e.g., proliferation, cytokine production, or enhanced phagocytosis), heat, redness, swelling, pain and/or loss of function in the intestine (small or large) of a subject, as defined herein. The cause of the intestinal inflammation can be due to physical damage, chemical substances, microorganisms, tissue necrosis, cancer, or other agents or conditions. Such intestinal inflammation can include acute inflammation, chronic inflammation, and recurrent inflammation. Acute inflammation is generally of relatively short duration, and last for from about a few minutes to about one to two days, although they can last several weeks. Characteristics of acute inflammation include increased blood flow, exudation of fluid and plasma proteins (edema) and emigration of leukocytes, such as neutrophils. Chronic inflammation, generally, is of longer duration, e.g., weeks to months to years or longer, and is associated histologically with the presence of lymphocytes and macrophages and with proliferation of blood vessels and connective tissue. Recurrent inflammation is inflammation which recurs after a period of time or which has periodic episodes. Some intestinal inflammation can fall within one or more categories. In some embodiments, the intestinal inflammation is colitis or colon cancer.

For administration of a therapeutic composition as disclosed herein (e.g., a broccoli-derived nanoparticle), conventional methods of extrapolating human dosage based on doses administered to a murine animal model can be carried out using the conversion factor for converting the mouse dosage to human dosage: Dose Human per kg=Dose Mouse per kg/12 (Freireich, et al., (1966) Cancer Chemother Rep. 50:219-244). Doses can also be given in milligrams per square meter of body surface area because this method rather than body weight achieves a good correlation to certain metabolic and excretionary functions. Moreover, body surface area can be used as a common denominator for drug dosage in adults and children as well as in different animal species as described by Freireich, et al. (Freireich et al., (1966) Cancer Chemother Rep. 50:219-244). Briefly, to express a mg/kg dose in any given species as the equivalent mg/sq m dose, multiply the dose by the appropriate km factor. In an adult human, 100 mg/kg is equivalent to 100 mg/kg×37 kg/sq m=3700 mg/m2.

Suitable methods for administering a therapeutic composition in accordance with the methods of the presently-disclosed subject matter include, but are not limited to, systemic administration, parenteral administration (including intravascular, intramuscular, and/or intraarterial administration), oral delivery, buccal delivery, rectal delivery, subcutaneous administration, intraperitoneal administration, inhalation, intratracheal installation, surgical implantation, transdermal delivery, local injection, intranasal delivery, and hyper-velocity injection/bombardment. Where applicable, continuous infusion can enhance drug accumulation at a target site (see, e.g., U.S. Pat. No. 6,180,082). In some embodiments, the broccoli-derived nanoparticles disclosed herein are administered orally.

Regardless of the route of administration, the compositions of the presently-disclosed subject matter are typically administered in amount effective to achieve the desired response. As such, the term “effective amount” is used herein to refer to an amount of the therapeutic composition (e.g., a broccoli-derived nanoparticle, and a pharmaceutically vehicle, carrier, or excipient) sufficient to produce a measurable biological response (e.g., a decrease in inflammation). Actual dosage levels of active ingredients in a therapeutic composition of the present invention can be varied so as to administer an amount of the active compound(s) that is effective to achieve the desired therapeutic response for a particular subject and/or application. Of course, the effective amount in any particular case will depend upon a variety of factors including the activity of the therapeutic composition, formulation, the route of administration, combination with other drugs or treatments, severity of the condition being treated, and the physical condition and prior medical history of the subject being treated. Preferably, a minimal dose is administered, and the dose is escalated in the absence of dose-limiting toxicity to a minimally effective amount. Determination and adjustment of a therapeutically effective dose, as well as evaluation of when and how to make such adjustments, are known to those of ordinary skill in the art.

For additional guidance regarding formulation and dose, see U.S. Pat. Nos. 5,326,902; 5,234,933; PCT International Publication No. WO 93/25521; Berkow et al., (1997) The Merck Manual of Medical Information, Home ed. Merck Research Laboratories, Whitehouse Station, N.J.; Goodman et al., (1996) Goodman & Gilman's the Pharmacological Basis of Therapeutics, 9th ed. McGraw-Hill Health Professions Division, New York; Ebadi, (1998) CRC Desk Reference of Clinical Pharmacology. CRC Press, Boca Raton, Fla.; Katzung, (2001) Basic & Clinical Pharmacology, 8th ed. Lange Medical Books/McGraw-Hill Medical Pub. Division, New York; Remington et al., (1975) Remington's Pharmaceutical Sciences, 15th ed. Mack Pub. Co., Easton, Pa.; and Speight et al., (1997) Avery's Drug Treatment: A Guide to the Properties, Choice, Therapeutic Use and Economic Value of Drugs in Disease Management, 4th ed. Adis International, Auckland/Philadelphia; Duch et al., (1998) Toxicol. Lett. 100-101:255-263.

hi some embodiments of the therapeutic methods disclosed herein, administering a broccoli-derived nanoparticle of the presently-disclosed subject matter reduces an amount of an inflammatory cytokine and/or an inflammatory chemokine in a subject. In some embodiments, the inflammatory cytokine is selected from interleukin-17A, tumor necrosis factor-alpha (TNF-α), interferon-γ (IFN-γ), or interleukin-22. In some embodiments, the inflammatory chemokine is selected from CCL20, CXCL1, and CCL25.

Various methods known to those skilled in the art can be used to determine a reduction in the amount of inflammatory cytokines and/or inflammatory chemokines in a subject. For example, in certain embodiments, the amounts of expression of an inflammatory cytokine in a subject can be determined by probing for mRNA of the gene encoding the inflammatory cytokine in a biological sample obtained from the subject (e.g., a tissue sample, a urine sample, a saliva sample, a blood sample, a serum sample, a plasma sample, or sub-fractions thereof) using any RNA identification assay known to those skilled in the art. Briefly, RNA can be extracted from the sample, amplified, converted to cDNA, labeled, and allowed to hybridize with probes of a known sequence, such as known RNA hybridization probes immobilized on a substrate, e.g., array, or microarray, or quantitated by real time PCR (e.g., quantitative real-time PCR, such as available from Bio-Rad Laboratories, Hercules, Calif.). Because the probes to which the nucleic acid molecules of the sample are bound are known, the molecules in the sample can be identified. In this regard, DNA probes for one or more of the mRNAs encoded by the inflammatory genes can be immobilized on a substrate and provided for use in practicing a method in accordance with the presently-disclosed subject matter.

With further regard to determining levels of inflammatory cytokines and chemokines in samples, mass spectrometry and/or immunoassay devices and methods can also be used to measure the inflammatory cytokines or chemokines in samples, although other methods can also be used and are well known to those skilled in the art. See, e.g., U.S. Pat. Nos. 6,143,576; 6,113,855; 6,019,944; 5,985,579; 5,947,124; 5,939,272; 5,922,615; 5,885,527; 5,851,776; 5,824,799; 5,679,526; 5,525,524; and 5,480,792, each of which is hereby incorporated by reference in its entirety. Immunoassay devices and methods can utilize labeled molecules in various sandwich, competitive, or non-competitive assay formats, to generate a signal that is related to the presence or amount of an analyte of interest. Additionally, certain methods and devices, such as biosensors and optical immunoassays, can be employed to determine the presence or amount of analytes without the need for a labeled molecule. See, e.g., U.S. Pat. Nos. 5,631,171; and 5,955,377, each of which is hereby incorporated by reference in its entirety.

Any suitable immunoassay can be utilized, for example, enzyme-linked immunoassays (ELISA), radioimmunoassays (RIAs), competitive binding assays, and the like. Specific immunological binding of the antibody to the inflammatory molecule can be detected directly or indirectly. Direct labels include fluorescent or luminescent tags, metals, dyes, radionucleotides, and the like, attached to the antibody. Indirect labels include various enzymes well known in the art, such as alkaline phosphatase, horseradish peroxidase and the like.

The use of immobilized antibodies or fragments thereof specific for the inflammatory molecules is also contemplated by the present invention. The antibodies can be immobilized onto a variety of solid supports, such as magnetic or chromatographic matrix particles, the surface of an assay plate (such as microtiter wells), pieces of a solid substrate material (such as plastic, nylon, paper), and the like. An assay strip can be prepared by coating the antibody or a plurality of antibodies in an array on a solid support. This strip can then be dipped into the test biological sample and then processed quickly through washes and detection steps to generate a measurable signal, such as for example a colored spot.

Mass spectrometry (MS) analysis can be used, either alone or in combination with other methods (e.g., immunoassays), to determine the presence and/or quantity of an inflammatory molecule in a subject. Exemplary MS analyses that can be used in accordance with the present invention include, but are not limited to: liquid chromatography-mass spectrometry (LC-MS); matrix-assisted laser desorption/ionization time-of-flight MS analysis (MALDI-TOF-MS), such as for example direct-spot MALDI-TOF or liquid chromatography MALDI-TOF mass spectrometry analysis; electro spray ionization MS (ESI-MS), such as for example liquid chromatography (LC) ESI-MS; and surface enhanced laser desorption/ionization time-of-flight mass spectrometry analysis (SELDI-TOF-MS). Each of these types of MS analysis can be accomplished using commercially-available spectrometers, such as, for example, triple quadropole mass spectrometers. Methods for utilizing MS analysis to detect the presence and quantity of peptides, such as inflammatory cytokines, in biological samples are known in the art. See, e.g., U.S. Pat. Nos. 6,925,389; 6,989,100; and 6,890,763 for further guidance, each of which are incorporated herein by this reference.

In some embodiments of the therapeutic methods of the presently-disclosed subject matter, administration of a broccoli-derived nanoparticle increases an amount of adenosine monophosphate-activated protein kinase (AMPK) signaling in a subject. In some embodiments, administration of the broccoli-derived nanoparticle reduces and amount of dendritic cell activation and/or increases an amount of dendritic cell tolerance in the subject. Measurements of such increases or reductions in activity can be done using any one of a number of methods known to those skilled in the art including, but not limited to, immunohistochemistry, polymerase chain reaction (PCR), and flow cytometry techniques.

With still further regard to the various therapeutic methods described herein, although certain embodiments of the methods disclosed herein only call for a qualitative assessment (e.g., the presence or absence of the expression of an inflammatory cytokine in a subject), other embodiments of the methods call for a quantitative assessment (e.g., an amount of increase in the level of an inflammatory cytokine in a subject). Such quantitative assessments can be made, for example, using one of the above mentioned methods, as will be understood by those skilled in the art.

The skilled artisan will also understand that measuring an increase or a reduction in the amount of a certain feature (e.g., cytokine levels) or an improvement in a certain feature (e.g., inflammation) in a subject is a statistical analysis. For example, a reduction in an amount of inflammatory cytokines in a subject can be compared to control level of inflammatory cytokines, and an amount of inflammatory cytokines of less than or equal to the control level can be indicative of a reduction in the amount of inflammatory cytokines, as evidenced by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Preferred confidence intervals of the present subject matter are 90%, 95%, 97.5%, 98%, 99%, 99.5%, 99.9% and 99.99%, while preferred p values are 0.1, 0.05, 0.025, 0.02, 0.01, 0.005, 0.001, and 0.0001.

Still further provided, in some embodiments, are methods for treating a colon cancer. In some embodiments, a method for treating a cancer is provided that comprises administering to a subject in need thereof an effective amount of a broccoli-derived nanoparticle. In some embodiments, administering the broccoli-derived nanoparticle decreases an amount of expression of COP9 signal some subunit 8 (CSN8). In some embodiments, administering the broccoli-derived nanoparticle reduces an amount of polyamine metabolism in an intestinal epithelial cell of the subject. In some embodiments, administering the broccoli-derived nanoparticle reduces an amount of rectal prolapse in the subject. In some embodiments of the therapeutic methods described herein, administering the broccoli-derived nanoparticle restores the gut microbiota in the subject. For example, in some embodiments, administering the broccoli-derived nanoparticle increases an amount of Bacteroidetes bacteria, reduces an amount of Actinobacteria bacteria, and/or reduces an amount of Proteobacteria bacteria present in the colon of the subject. In some embodiments, administering the broccoli-derived nanoparticle increases an amount of an antimicrobial peptide in an intestinal epithelial cell of the subject. Again, such assessments can be made using one of the above mentioned methods as well as other methods known to those skilled in the art.

Even further provided, in some embodiments of the presently-disclosed subject matter are methods for screening for compounds useful for treating a colon cancer. In some embodiments, a method for screening for a compound useful for treating a colon cancer is provided that comprises the steps of: providing an intestinal epithelial cell; contacting the intestinal epithelial cell with a test compound; measuring an amount of expression of COP9 signalsome subunit 8 (CSN8) in the intestinal epithelial cell; and identifying the test compound as useful for treating colon cancer is the amount of CSN8 expression in the intestinal epithelial cell is decreased relative to a control amount of CSN8 expression in a subject.

As used herein, the term “subject” includes both human and animal subjects. Thus, veterinary therapeutic uses are provided in accordance with the presently disclosed subject matter. As such, the presently-disclosed subject matter provides for the treatment of mammals such as humans, as well as those mammals of importance due to being endangered, such as Siberian tigers; of economic importance, such as animals raised on farms for consumption by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses. Also provided is the treatment of birds, including the treatment of those kinds of birds that are endangered and/or kept in zoos, as well as fowl, and more particularly domesticated fowl, i.e., poultry, such as turkeys, chickens, ducks, geese, guinea fowl, and the like, as they are also of economic importance to humans. Thus, also provided is the treatment of livestock, including, but not limited to, domesticated swine, ruminants, ungulates, horses (including race horses), poultry, and the like.

The practice of the presently-disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

The presently-disclosed subject matter is further illustrated by the following specific but non-limiting examples.

### EXAMPLES

### Material and Methods for Examples 1-7

Isolation and characterization of broccoli-derived small nanoparticles (BDN). Fresh broccoli was purchased from a local market and washed 3× with PBS and ground in a mixer (Osterizer 12-speed blender) at the highest speed for 10 min (pause 1 min for every 1 min blending). Broccoli juice was then sequentially centrifuged at 1,000 g for 10 min, 3,000 g for 20 min and 10,000 g for 40 min. After 10,000 g centrifugation, the pellet was resuspended in PBS and referred to as microparticles. The supernatant was then centrifuged at 100,000 g for 90 min and the pellet depleted supernatants were saved for isolation of broccoli-derived nanoparticles (BDN) using a nanoparticle isolation system described previously. In brief, the sample was continually pumped using a pressure-regulated pump into a Biomax-500 column. The molecules that were greater than 500 kDas were retained and collected for sucrose purification. The protein concentration of the washed samples after sucrose purification was determined using a BCA assay kit (Thermo Scientific).

Animals and treatments. C57BL/6J, OT-II mice in a C57BL/6 background, and Rag1^{-/-} mice in a C57BL/6J background were obtained from Jackson Laboratories. AMPKα1^{-/-} 129S2/SvPas (AMPKα1 KO) mice were generated as previously described. All animal procedures were approved by the University of Louisville Institutional Animal Care and Use Committee.

Adoptive T cell transfer model of chronic colitis. T-cell mediated colitis was induced in Rag1^{-/-} mice by adoptively transferring naïve CD4⁺CD25⁻CD62L⁺ T cells from C57BL/6 mice that were isolated using a FACS Aria II flow cytometer (BD Biosciences). The B6 Rag1^{-/-} recipients were given 5×10⁵ CD4⁺CD25⁻CD62L⁺ T cells via intraperitoneal injection, and were euthanized at 6-7 weeks after transfer. For the adoptive transfer of BDN experiment, some recipient mice also received, by oral administration, 250 μg of BDNs or PBS once every 3 days for 6 weeks after the transfer of CD4⁺CD25⁻CD62L⁺ T cells. Mouse colitis phenotypes were analyzed and scored as detailed previously.

Chemically induced colitis models. Colitis was induced in 8- to 12-week-old C57BL/6J mice by the addition of 2.5% (wt/vol) DSS (36-50 KD molecular weight, MP Biomedicals, OH) in their drinking water. C57BL/6 mice were given BDNs orally (250 μg/mouse in PBS) before (every day for 10 d) and after (every 2 days for 12 d) administration of drinking water (H2O) or water containing DSS (2.5% DSS). Body weights, stool consistency and GI bleeding were monitored daily. Clinical scores and colonic damage scores were estimated as detailed previously. Colons were collected immediately after sacrifice, and mucosa was scraped to isolate total RNA or proteins.

Agonistic αCD40 Model of Colitis. B6 Rag1^{-/-} mice were injected intraperitoneally with 200 μg of rat anti-mouse-CD40 (FGK45, BioXcell, West Lebanon, N.H.) or with rat IgG2a. Mice were given BDNs orally (250 μg/mouse in PBS) before (every day for 7 d) and after (every 2 days for 6 d) injection of antibody. Mice were weighed daily and killed at day 4 or day 7.

Disease monitoring and scoring. Mice were weighed daily and monitored for appearance and signs of soft stool and diarrhea. A combinatorial index of disease, or disease activity index (DAI), defined as stool blood, stool form, and weight loss was used to analyze the degree of colitis. Histologic grades and inflammation was assessed with a modified version of a previously described scoring system. Each sample was graded semi-quantitatively from 0 to 3 for the four following criteria: degree of epithelial hyperplasia and goblet cell depletion; leukocyte infiltration in the lamina propria; area of tissue affected; and the presence of markers of severe inflammation such as crypt abscesses, submucosal inflammation, and ulcers. Scores for each criterion were added to give an overall inflammation score for each sample of 0-8.

Reagents, antibodies and flow cytometry. Lipophilic fluorescent dye BODIPY 493/503 (D3922) was purchased from Thermofisher. Rat anti-mouse-CD40 (FGK45) was purchased from BioXcell (West Lebanon, N.H.). Sulforaphane was purchased from Sigma-Aldrich. For analysis of surface markers, cells were stained in PBS containing 2% (wt/vol) BSA. Intracellular staining of the transcription factors Foxp3 was performed using the Foxp3 Fix/Perm Buffer Set (eBioscience). For detection of intracellular cytokines, cells were first stimulated for 4 h with 50 ng/ml PMA and 1 μg/ml ionomycin in the presence of Brefeldin A (5 μg/ml; All obtained from Sigma), followed by staining for surface markers. Cells were then fixed and permeabilized using the Foxp3 Fix/Perm Buffer Set (eBioscience) and stained for intracellular cytokines. The following antibodies were used at a dilution of 1/200-1/600: PerCP-Cy5.5, PE-, FITC- or APC-labeled anti-IL-17A (TC11-18H10.1), PE- or APC-labeled anti-IL-4 (11B11, eBioscience), PE- or APC-labelled anti-IL-10 (JES5-16E3), APC- or PE-Cy7-labeled anti-IFN (XMG1.2),PE-labeled anti-Foxp3 (FJK-16s, eBioscience), PE-, FITC- or APC-labeled anti-CD11b (M1/70), PE-, FITC- or APC-labeled anti-CD4 (RM4-5), PE-Cy7-labeled anti-CD3 (145-2C11), PE-anti-Gr-1 (RB6-8C5), PE- or FITC-labeled anti-mouse Ly6G (1A8), APC-conjugated CD45.2 (104), PE-conjugated anti-CD45.1 (A20), FITC-, PerCP-Cy5.5 or Pacific Blue-labelled anti-CD45 (30-F11), PE-anti-CCR4 (2G12), PE- or FITC-labelled anti-CCR9 (CW-1.2). All antibodies were obtained from Biolegend unless otherwise noted. Flow cytometry data were acquired on a 5-color FACScan (Becton Dickinson) and analyzed using FlowJo software (Treestar). Cell sorting was performed using a FACSAria II.

Histology and immunohistochemistry. Tissue specimens were fixed in 10% formalin, dehydrated, and then embedded in paraffin. Tissue samples were cut at 5 μm thicknesses and stained with hematoxylin and eosin. For immunofluorescence analysis, tissue sections were subjected to antigen retrieval by boiling the slides in Antigen Unmasking Solution (Vector Laboratories) for 10 minutes according to instructions. Sections were then blocked for 1 hour at 22° C. with 5% BS A in PBS and incubated overnight at 4° C. with the primary antibodies, i.e., rabbit polyclonal Ki67 and phospho-AMPKα (Thr172) (40H9) antibody from Cell Signaling used at a dilution of 1/250, mouse monoclonal anti-E-cadherin, anti-CD11c, F4/80 and CD11b were purchased from BD Bioscience (San Jose, Calif.) and used at a dilution of 1/100. Primary antibodies were detected by Alexa Fluor 488,594 or 647 conjugated goat anti-mouse, anti-rabbit IgG and anti-rat (1:600, Invitrogen). Tissues were counterstained with DAPI and images were captured on a Zeiss LSM 510 confocal microscope equipped with a digital image analysis system (Pixera). For immunohistochemistry analysis of dendritic cells or macrophages, frozen sections were stained with CD11c or F4/80 (BM8, ebioscience). OCT (Sakura Finetek)-embedded tissue cryosections (9 μm-thick) were also stained with anti-phospho-S6 (Ser235/236,D57.2.2E), or p70S6 Kinase (Cell Signaling) antibody as primary antibody, followed by staining with horseradish peroxidase-conjugated anti-IgG second antibodies. Antigens were then visualized with 3,3′-diaminobenzidine substrate (Vector Laboratories) and scanned using an Aperio Imagescope.

RNA extraction and PCR. Total RNA was isolated from the tissue or lymphocytes of MLNs, small intestine and colon using the Qiagen RNeasy RNA isolation Kit and was used to synthesize cDNA. RNA (1 μg) was reverse-transcribed with Superscript in and random primers (Invitrogen). For quantitation of genes of interest, cDNA samples were amplified in a CFX96 Realtime System (Bio-Rad Laboratories, Hercules, Calif., USA) using SYBR Green Master Mix (Invitrogen) and specific primers (Table 1) according to the manufacturer's instructions. Fold changes in mRNA expression between treatments and controls were determined by the 8CT method as described. Results for each sample were normalized to the concentration of GAPDH mRNA measured in the same samples and expressed as fold increase over baseline levels, which are set at a value of 1. Differences between groups were determined using a two-sided Student's t-test and one-way ANOVA. Error bars on plots represent±SEM, unless otherwise noted. All primers were purchased from Eurofins MWG Operon.

TABLE 1
Primers used for Real-time PCR

| Gene | | |
|---|---|---|
| name | Forward primer | Reverse primer |
| IL-17F | AATTCCAGAACCGCTCCAG | TTGATGCAGCCTGAGTGTCT |
| | (SEQ ID NO: 1) | (SEQ ID NO: 2) |
| GAPDH | AGGTCATCCCAGAGCTGAACG | ACCCTGTTGCTGTAGCCGTAT |
| | (SEQ ID NO: 3) | (SEQ ID NO: 4) |
| β-Actin | ACGGCCAGGTCATCACTATTC | AGGAAGGCTGGAAAAGAGCC |
| | (SEQ ID NO: 5) | (SEQ ID NO: 6) |
| TNF-α | TCTATGGCCCAGACCCTCAC | GACGGCAGAGAGGAGGTTGA |
| | (SEQ ID NO: 7) | (SEQ ID NO: 8) |
| IL-22 | TTGAGGTCGTCCAACTTCCAGCA | AGCCGGACGTCGTGTTGTTA |
| | (SEQ ID NO: 9) | (SEQ ID NO: 10) |
| IL-17A | TTTAACTCCCTTGGCGCAAAA | CTTTCCCTCCGCATTGACAC |
| | (SEQ ID NO: 11) | (SEQ ID NO: 12) |
| CXCL1 | AGCCACCCGCTCGCTTCTCTGTG | AGCCTCGCGATTCTTGAGTGTGG |
| | (SEQ ID NO: 13) | (SEQ ID NO: 14) |
| SDF-1α | CTGTGCCCTTCAGATTGTTG | TCAGCCTTCCTCGGGGGTCT |
| | (SEQ ID NO: 15) | (SEQ ID NO: 16) |
| CCL2 | GTTGGCTCAGCCAGATGCA | AGCCTACTCATTGGGATCATCTTG |
| | (SEQ ID NO: 17) | (SEQ ID NO: 18) |
| IL-10 | TTTGAATTCCCTGGGTGAGAA | GGAGAAATCGATGACAGCGC |
| | (SEQ ID NO: 19) | (SEQ ID NO: 20) |
| IFN-γ | TCAGCAACAGCAAGGCGAAAAAGG | CCACCCCGAATCAGCAGCGA |
| | (SEQ ID NO: 21) | (SEQ ID NO: 22) |
| CCL20 | GTGGGTTTCACAAGACAGATG | TTTTCACCCAGTTCTGCTTTG |
| | (SEQ ID NO: 23) | (SEQ ID NO: 24) |
| CCL25 | GCTTTTTGCCTGCCTGGTTG | TCAGTCTGAGAGTCTGAGGC |
| | (SEQ ID NO: 25) | (SEQ ID NO: 26) |
| IL-10 | TTTGAATTCCCTGGGTGAGAA | GGAGAAATCGATGACAGCGC |
| | (SEQ ID NO: 19) | (SEQ ID NO: 20) |
| IL-6 | GAGAGGAGACTTCACAGAGGATAC | GTACTCCAGAAGACCAGAGG |
| | (SEQ ID NO: 27) | (SEQ ID NO: 28) |
| IL-4 | GAGACTCTTTCGGGCTTTTC | TGATGCTCTTTAGGCTTTCCA |
| | (SEQ ID NO: 29) | (SEQ ID NO: 30) |
| IL-23p19 | GGTGGCTCAGGGAAATGT | GACAGAGCAGGCAGGTACAG |
| | (SEQ ID NO: 31) | (SEQ ID NO: 32) |
| CXCL2 | CGCTGTCAATGCCTGAAG | GGCGTCACACTCAAGCTCT |
| | (SEQ ID NO: 33) | (SEQ ID NO: 34) |
| CXCL10 | CCCACGTGTTGAGATCATTG | TCCATCACAGCACCGGG |
| | (SEQ ID NO: 35) | (SEQ ID NO: 36) |
| IL-12 | TACTAGAGAGACTTCTTCCACAACA | TCTGGTACATCTTCAAGTCCTCATAGA |
| p35 | AGAG (SEQ ID NO: 37) | (SEQ ID NO: 38) |
| IL-12 | GACCATCACTGTCAAAGAGTTTCTA | AGGAAAGTCTTGTTTTTGAAATTTTTTAA |
| p40 | GAT (SEQ ID NO: 39) | (SEQ ID NO: 40) |
| CCR6 | CCTCACATTCTTAGGACTGGAGC | GGCAATCAGAGCTCTCGGA |
| | (SEQ ID NO: 41) | (SEQ ID NO: 42) |
| CCR9 | CACAGACTTCACAAGCCCTA | GTACAAGGGTGGGAGGAAAT |
| | (SEQ ID NO: 43) | (SEQ ID NO: 44) |
| TGF-β1 | TGACGTCACTGGAGTTGTACGG | GGTTCATGTCATGGATGGTGC |
| | (SEQ ID NO: 45) | (SEQ ID NO: 46) |
| Aldh1a2 | ATGCGGATTGCCAAGGAGG | TGAAGACAGCTGCTACAAGTCC |
| | (SEQ ID NO: 47) | (SEQ ID NO: 48) |
| CCR4 | AACAGAGCAGTGCGCATGAT | CGTTGTACGGCGTCCAGAA |
| | (SEQ ID NO: 49) | (SEQ ID NO: 50) |
| CCR2 | GAAGGAGGGAGCAGTGTGTACAT | CCCCCACATAGGGATCATGA |
| | (SEQ ID NO: 51) | (SEQ ID NO: 52) |

Enzyme linked immunosorbent assay (ELISA). The quantity of IL-17A, IL-6, TNF-α, IL-10, IFN-γ (eBioscience), CXCL1 and CCL2 (R&D Systems) were determined in culture supernatants, serum and tissue using ELISA kits according to the manufacturer's instructions. The sensitivity of the assay was less than 20 μg/ml.

Cells and cell culture conditions. Cells were maintained in DMEM supplemented with 10% fetal bovine serum (FBS) and 100 U/ml penicillin/streptomycin. All cells were grown in a humidified atmosphere of 5% CO₂ at 37° C. For dendritic cells and BDN derived lipid-mediated Th1/Th2 proliferation and differentiation, naïve CD4⁺CD25⁻CD62L⁺ T lymphocytes were cultured for 5 days with anti-CD3 (5 μg/ml, 2C11, Bio X cell), anti-CD28 (2 μg/ml, 37.51; Bio X Cell), IL-2 (10 ng/ml), anti-IL-4 (10 μg/ml, for Th1) or anti-IFN-γ (10 μg/ml, for Th2) in the presence of BDNs (20 μg/ml) or BDN-lipid (200 μM), followed by stimulation with PMA and ionomycin in the presence of Brefeldin A (10 μg/ml Sigma). Intracellular cytokine production on CD4⁺ T cells was determined by flow cytometry. For DC-T cell co-cultures, 1×10⁵ DC and 5×10⁵ OT-II T cells were mixed in the presence of cognate peptide (5 μg/ml; OVA) and/or BDN (20 μg/ml) or BDN-lipid (200 μM). After 4 d of culture, live T cells were collected and stimulated with PMA (phorbol 12-myristate 13-acetate, 50 ng/ml) and ionomycin (1 μg/ml Sigma) plus Brefeldin A (10 μg/ml Sigma) for intracellular cytokine staining or for mRNA analysis. mRNA was assessed by RT-PCR and supernatants were used for cytokine measurement by ELISA.

Isolation of bacterial antigens from the cecum (CBA) for in vitro cell culture. Fresh caecal content was collected and carefully resuspended in PBS. The obtained suspension was centrifuged at 400 g for 5 min to remove larger particles from bacteria. Bacterial suspensions were then lysed by physical disruption through sonication. The protein concentration of the lysate was quantified using a Bradford protein assay. We used 20 μg/ml total CBA for in vitro stimulation.

Uptake of BDN in vivo. To monitor nanoparticle trafficking and uptake in vivo, BDN were labeled with the PKH26 red fluorescent dye using a commercially available kit (Sigma-Aldrich) and according to a previously described protocol. Mice with/without colitis were orally administered 250 μg PKH26-labeled BDNs. Eighteen to twenty-four h after transfer the mice were sacrificed and the liver, MLN and spleen tissues were collected. Single-cell suspensions of each tissue were prepared in RPMI 1640 medium and subjected to FACS analysis. The percentages of cells containing BDNs were determined by counting red fluorescent-positive cells. For analysis of uptake of BDN by dendritic cells or macrophages, frozen sections of intestine and MLN were stained with CD11c or F4/80 (BM8, ebioscience).

Lipid extraction, TLC and lipidomic analysis. Total lipid extraction from BDNs was performed according to the method of Bligh and Dyer, and the lipids were dissolved in chloroform or methanol for analysis. For TLC analysis, lipids extracted as described above and sulforaphane (SFN, Sigma-Aldrich, 10 pMol) were applied on a Silic gel 60 Å TLC plate (Whatman) and developed in a mixture of hexane/ethyl acetate/formic acid=55:40:5. Analysis of lipids extracted from broccoli, broccoli microparticles, and BDNs was accomplished by developing the TLC with a mixture of toluene-ethyl acetate (3:1, v/v). Developed plates were initially air-dried, then sprayed with CuSO₄-phosphoric acid reagent (10% CuSO₄ in 8% phosphoric acid), and followed by charring at 100° C. for 10 min. To knockout SFN from BDN lipids, duplicated BDN-derived lipid samples were loaded on the same TLC plate. A standard control of SFN (Sigma) was loaded next to BDN lipid samples and used to determine the position of SFN in the BDN lipids loaded on the same TLC plate. After separation on the TLC plate, one of the duplicate BDN-derived lipid samples and the SFN standard were developed as a reference for the location of BDN SFN on the TLC plate. The band that had migrated to the same position as the standard SFN was removed for high-performance liquid chromatography (HPLC) analysis and the remaining fractions of the BDN lipids in the TLC were collected and extracted with 2 mL of chloroform/methanol (1:1, v/v) and 0.9 mL water. The organic phase samples were aliquoted and dried by heat under nitrogen (0.2 psi). Total lipids were determined using the phosphate assay as described previously. For assembling liposome-like nanoparticles (LN), the dried lipids were immediately suspended in distilled water (150-200 μL). After bath-sonication (FS60 bath sonicator, Fisher Scientific, Pittsburgh, Pa.) for 5 min, an equal volume of buffer (308 mM NaCl, 40 mM HEPES, pH 7.4) was added and sonicated for another 5 min. The charges and sizes of liposome-like nanoparticles were examined using a method as described previously.

Western blot analysis. Tissue or cells were disrupted in lysis buffer containing 1% Triton X-100, 0.1% SDS, 150 mM NaCl, 50 mM Tris-HCl, 1 mM EDTA, 1 mM EGTA, 5 mM sodium molybdate and 20 mM phenylphosphate with protease and phosphatase inhibitors (1 mM PMSF, 10 μg ml⁻¹ aprotinin, 20 μg ml⁻¹ leupeptin, 20 μg ml⁻¹ pepstatin A, 50 mM NaF and 1 mM sodium orthovanadate) for 30 min on ice. The samples were centrifuged (16,000 g, 10 min, 4° C.) and the resulting supernatants transferred to fresh tubes. Protein lysates were quantitated using a Bio-Rad protein kit (Bio-Rad) and 50-100 μg of lysates were separated on 10% SDS polyacrylamide gels and transferred to a nitrocellulose membrane. Rabbit anti-phospho-Stat3 (727), phospho-Stat3 (705), phospho-AKT (ser473), anti-phospho-S6 (Ser235/236,D57.2.2E), phospho-p70S6 Kinase (Thr421/Ser424) or STAT3 and actin were purchased from Cell Signaling Technology (Danvers, Mass.) and used at a dilution of 1/1,000. Membranes were probed with specific antibodies and protein quantity visualized using an Odyssey instrument (Li-CoR Bioscience). Images have been cropped for presentation.

Isolation of lamina propria lymphocytes (LPLs) and flow cytometry analysis. The method used for isolation of LPLs has been previously described. In brief, fat tissues and Peyer's patches (PPs) were removed from small intestine. The intestine was open and cut in pieces 1-cm long and incubated in an HBSS solution containing 5 mM EDTA and 10 mM Hepes) for 30 min at 37° C. with slow rotation (180 r.p.m. min⁻¹). Pieces were then further cut and incubated in an HBSS solution containing 0.5 mg ml⁻¹ DNase I (Roche) and 1 mg ml⁻¹ Collagenase type IV (Worthington). Finally, the solution containing digested tissue was passed through a 100-μm cell strainer and LPLs were recovered at the interface of the 40 and 80% Percoll (GE Healthcare) solutions. For flow cytometry analysis, the cells were labelled using standard procedures described above.

Adoptive transfer of cells. For transfer of monocytes, bone marrow cells were harvested from the femurs and tibias of mice and sorted for CD115⁺ckit⁻CD11c⁻CD11b⁺Gr1⁺ or Gr1⁻ monocytes. C57BL/6 CD45.2 Rag1^{-/-} mice were given BDNs orally (250 μg/mouse in PBS) before (every day for 10 d) and after (every 2 days for 8d) administration of water containing DSS (2.5% DSS). FACS sorted monocytes from B6 CD45.1 mice were i.v. injected at 3*10⁶ cells into colitic B6 CD45.2 Rag1^{-/-} mice that had received 2.5% DSS for 8 days. Thirty-six hours after monocyte transfer, tissues were collected for the detection of CD45.1⁺CD11b⁺ DCs by FACS analysis. For transfer of DCs, BMDCs were treated with CBA (20 μg/ml) for 48 hr in the presence of DMSO, BDN-derived lipid, LN-SFN^{-/-} or LN-SFN^{+/+} and were injected i.v. (2×10⁶) into each recipient (C57B1/6) mouse at days −1 and +3 of DSS treatment. Colitis was assessed as described above.

Generation of mouse and human DCs. For the generation of mouse DCs, bone marrow cells were harvested from the femurs and tibias of mice and cultured in 6-well tissue culture plates (Costar) for 8 days in complete medium supplemented with 20 ng/ml GM-CSF and 10 ng/ml IL-4. BDNs, BDN-derived lipid or SFN was added to some wells on day 0. To obtain human DCs, peripheral blood mononuclear cells were isolated from healthy donors by Ficoll-Hypaque Plus (GE Healthcare). These studies were approved by the Institutional Review Board of University of Louisville. Monocytes were purified by a discontinuous Percoll gradient (GE Healthcare) and positive selection with the Monocyte Isolation kit (purity, >90%; Miltenyi Biotec). Monocytes were cultured in RPMI-1640 complete medium supplemented with 10% (vol/vol) heat-inactivated PCS, gentamicin (40 g/ml), 2-mercaptoethanol (50 μM) and L-glutamine (2 mM; all from Gibco) containing IL-4 (5 ng/ml; Sigma) and recombinant human GM-CSF (35 ng/ml; Sigma) with or without SFN (10 μM). In some experiments, DCs were exposed for 24 h to LPS (100 ng/ml) with or without SFN.

Statistical analysis. Values are shown as S.E.M. except otherwise indicated. Comparison of multiple experimental groups was performed by two-way Analysis of Variance test. A t-test was used to compare the means of two groups. P values of <0.05 were considered to be statistically significant. Sample sizes are calculated to allow significance to be reached.

### Example 1—Broccoli-Derived Nanoparticles (BDN) Prevent Mouse Colitis

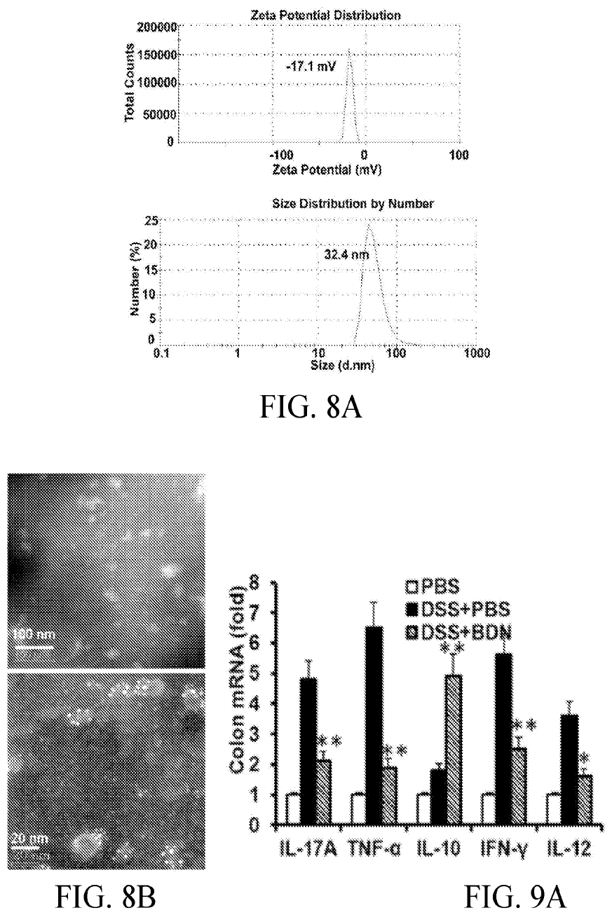

It is appreciated that broccoli has anti-inflammatory effects, and edible plant nanoparticles have been characterized in a number of plants. Whether broccoli nanoparticles have anti-inflammatory effects was not known. In the following study, broccoli-derived nanoparticles (BDN) were isolated according to a protocol previously established. In brief, after a 100,000 g centrifugation for 1 h, the supernatant was harvested from homogenized broccoli using a sequential centrifugation method. BDNs from the supernatant were isolated with a simple column filtration method as described. The size distribution of the BDNs was determined using a nanosizer (FIG. 8A) and confirmed by electron microscopy (FIG. 8B). The size distribution of the isolated BDNs ranged from 18.3 to 118.2 ran in diameter, with an average diameter of 32.4 ran. Zeta potential measurements indicated that BDNs had a negative zeta potential value ranging from −39.2 to −2.62 mV, with an average zeta potential value of −17.1 mV.

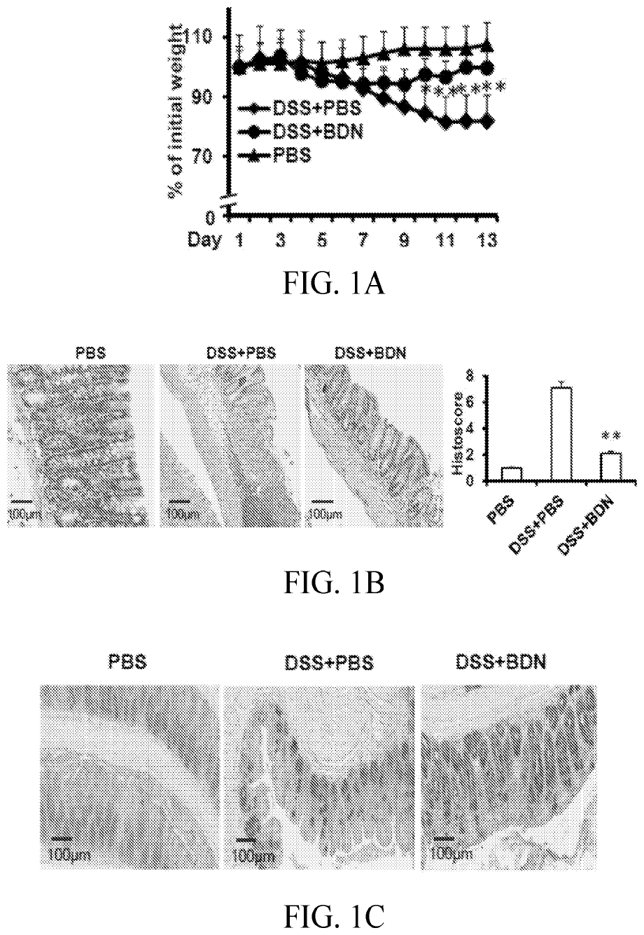

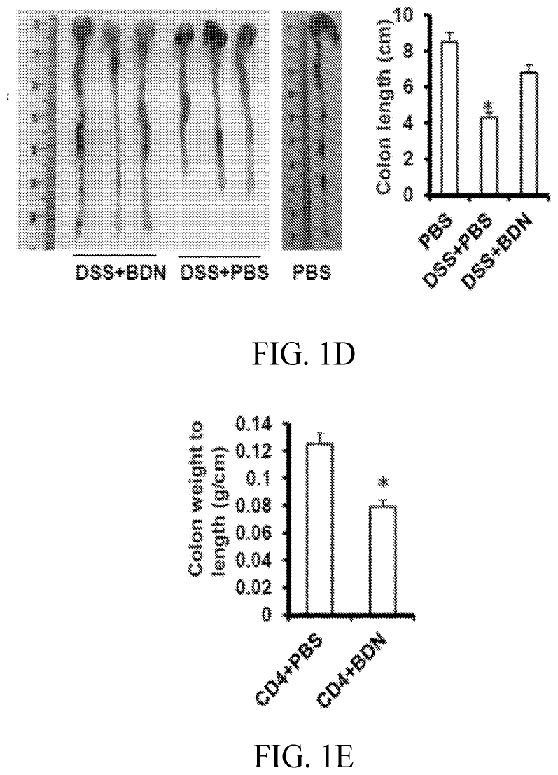

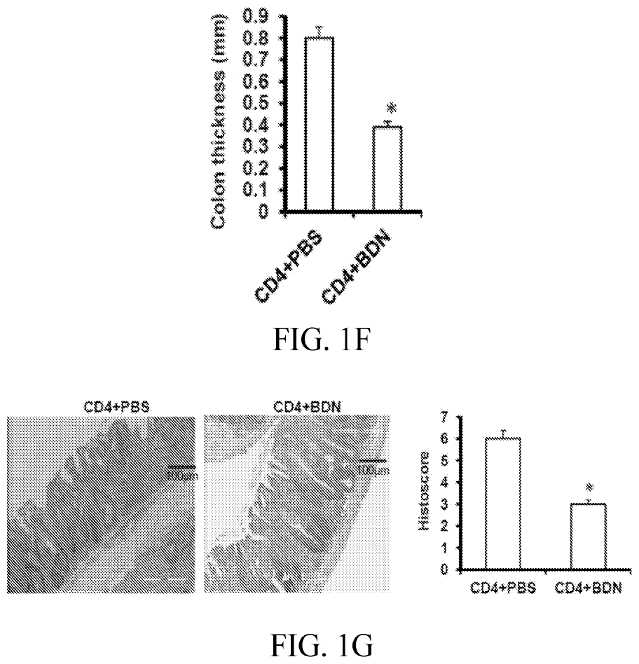

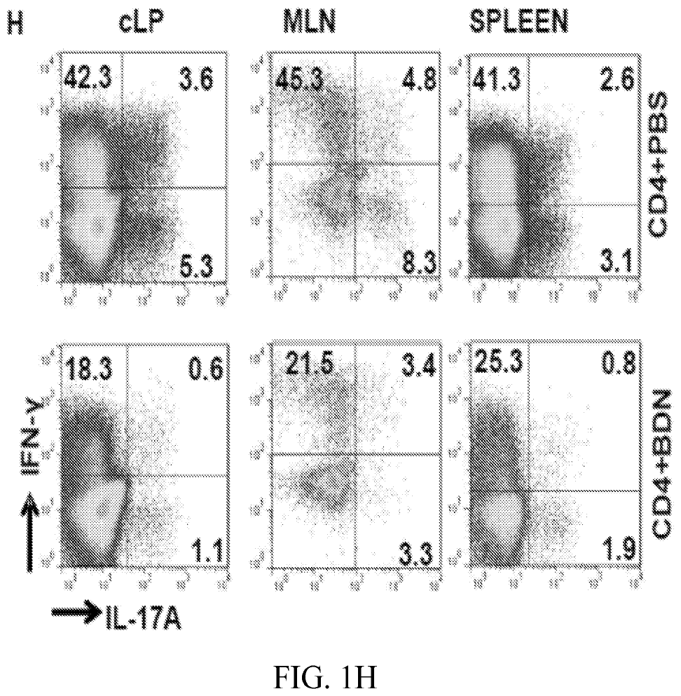

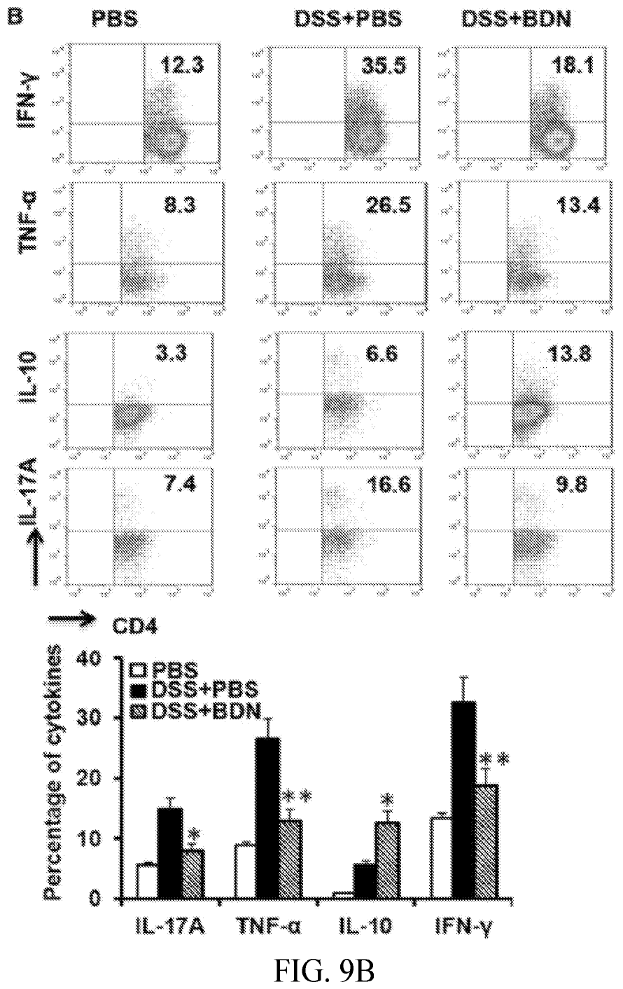

To explore whether BDNs played a role in prevention of experimental colitis, two mouse colitis models were used for testing the hypothesis. Mice were gavaged with BDNs for 10 days before 2.5% dextran sulphate sodium (DSS) was provided in drinking water. Mice treated with only DSS had a gradual weight loss (FIG. 1A); whereas BDN treatment prevented DSS-induced weight loss (FIG. 1A). BDN-treated mice also had reduced inflammatory infiltrate in the mucosa (FIG. 1B), more colonic goblet cells (FIG. 1C), and significantly less colonic shortening (FIG. 1D) when compared to mice receiving only DSS. A dysregulated cytokine profile is one of the features of DSS-induced colitis, and thus, it was further evaluated whether the BDN treatment could affect the profile of proinflammatory and anti-inflammatory cytokines. DSS administration significantly increased the expression of IFN-γ, IL-17A, and tumor necrosis factor (TNF)-α in colonic tissues (FIG. 9A). In contrast, BDN treatment led to a reduction of DSS induced INF-α, IL-17A and IFN-γ and an increased expression of IL-10 (FIG. 9A). Flow cytometry analysis of lamina propria lymphocytes (LPL) confirmed that BDNs reduced the percentages of IFN-γ, TNF-α and IL-17A-positive CD4 T cells and enhanced the percentage of IL-10-expressing cells (FIG. 9B).

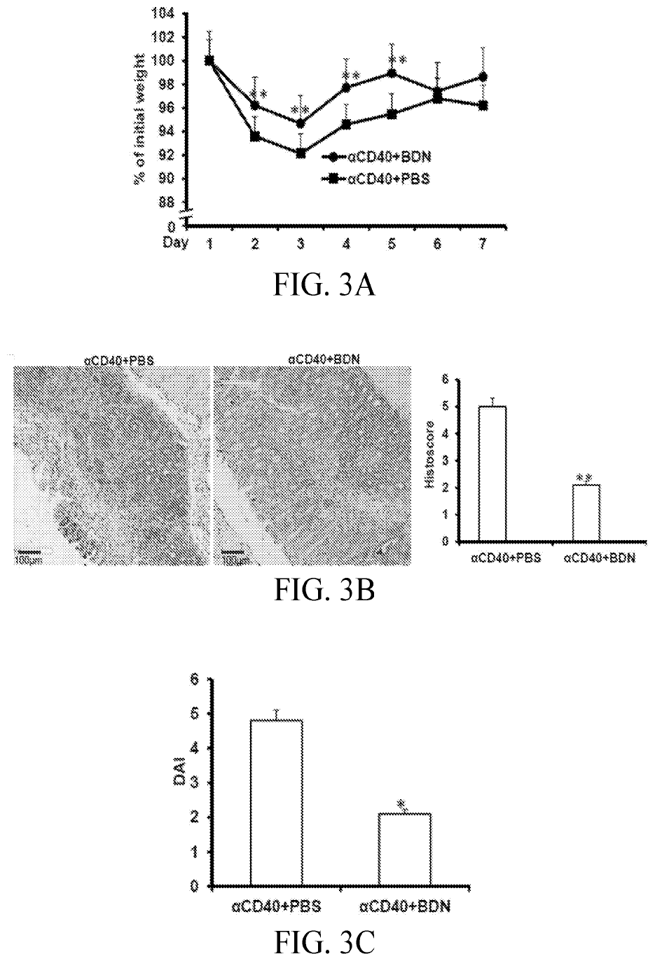

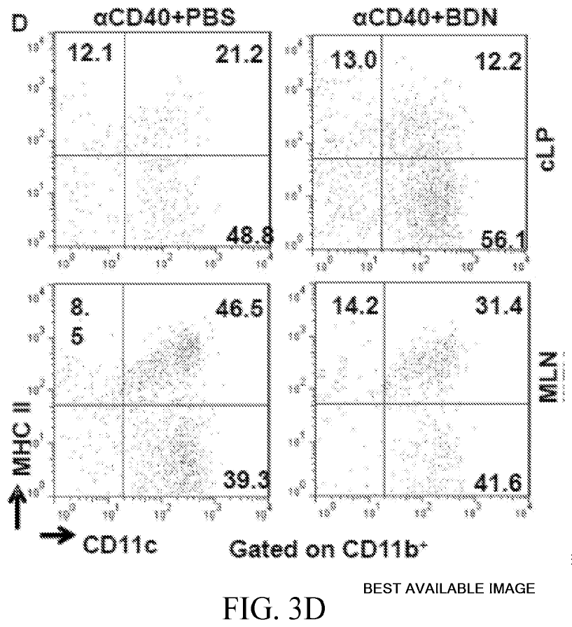

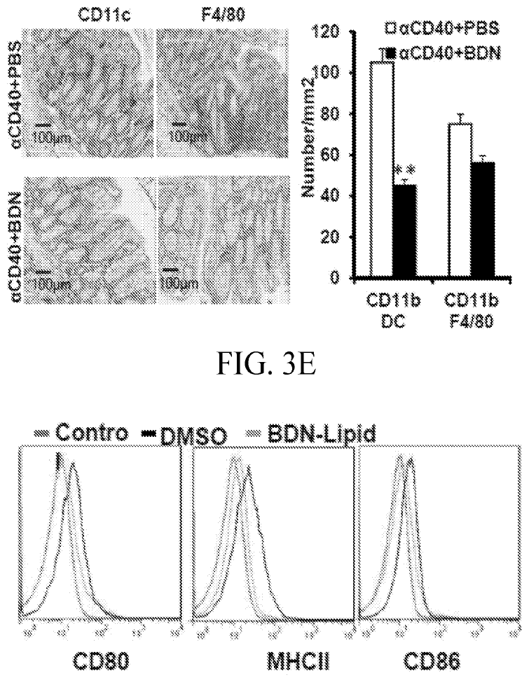

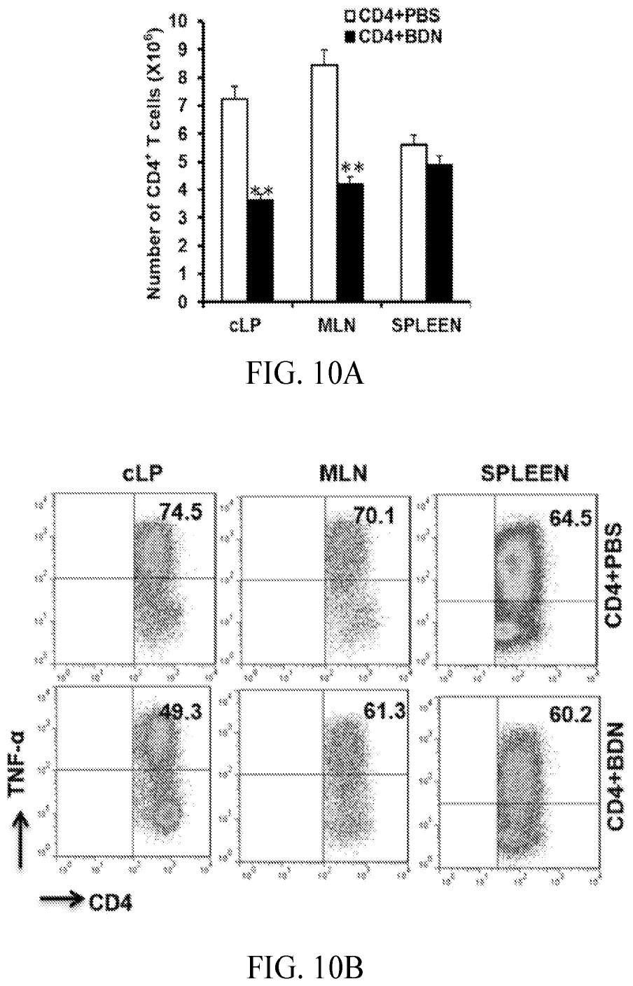

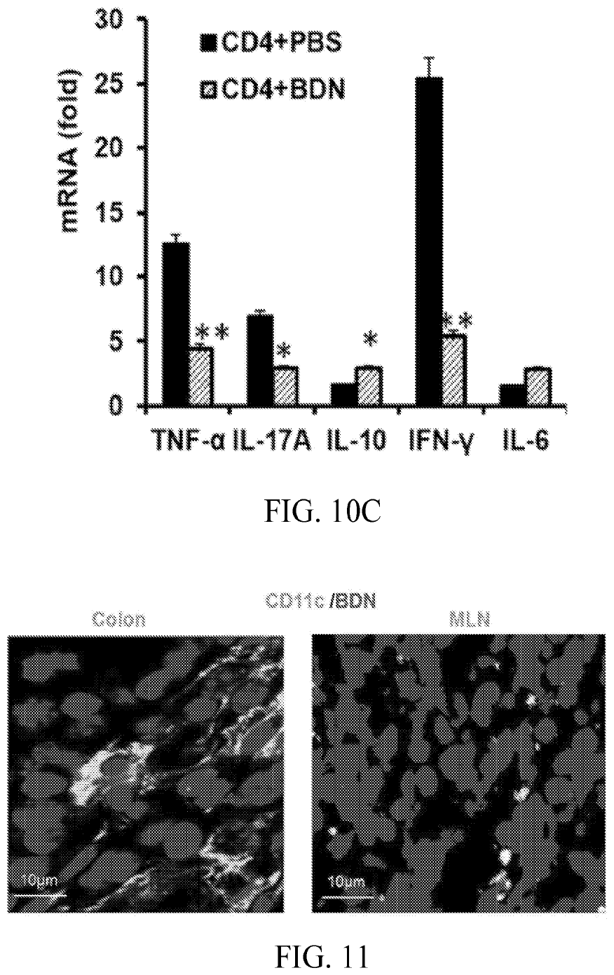

Both innate and adaptive immunity play a role in human inflammatory bowel diseases (IBD), including Crohn's disease (CD) and ulcerative colitis (UC). DSS-induced acute colitis, one of the most popular murine colitis models, is considered a T cell independent model. Dysregulated CD4⁺ T cells in adaptive immunity have been postulated to play an important role in the pathogenesis of IBD. It was next determined whether BDNs had anti-inflammatory effects in a T cell-dependent model of colitis induced by the adoptive transfer of naïve T cells into Rag1-deficient mice. BDNs were orally administered 1 week after transfer of T cells. As expected, 4 weeks after T-cell reconstitution, mice manifested clinical signs of colitis (FIGS. 1E-1G). Surprisingly, BDN treated mice failed to develop colitis evidenced by decreased ratios of colon weight/length, mucosal wall thickness and histological score (FIGS. 1E-1G). A significantly lower number of CD4⁺ T cells was also found in MLNs and LPL in BDN-treated recipient mice at day 35 after T-cell transfer (FIG. 10A). Consistent with these results, decreased percentages of TNF-α-, IFN-γ-, IL-17A-, and IFN-γ/IL-17A-T cells were detected in colonic tissue, MLNs and spleens of BDN-treated mice compared to that of PBS-treated mice (FIG. 1H, FIG. 10B). Real-time PCR results confirmed that amounts of TNF-α, IFN-γ and IL-17A mRNA in colon were decreased in BDN-treated mice compared with PBS-treated mice (Supplementary FIG. 3C). Together, the data from both colitis models indicate that BDNs prevent mouse colonic inflammation and colitis and inhibit pro-inflammatory cytokine induction in the MLNs and the colon as well as Th1 cell generation.

### Example 2—BDNs Inhibit the Activation of Intestinal DCs as Well as Recruitment of Monocytes in Mouse Colitis

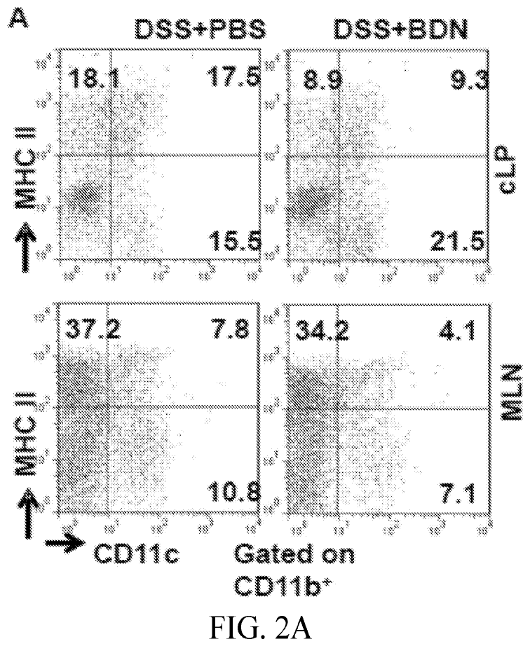

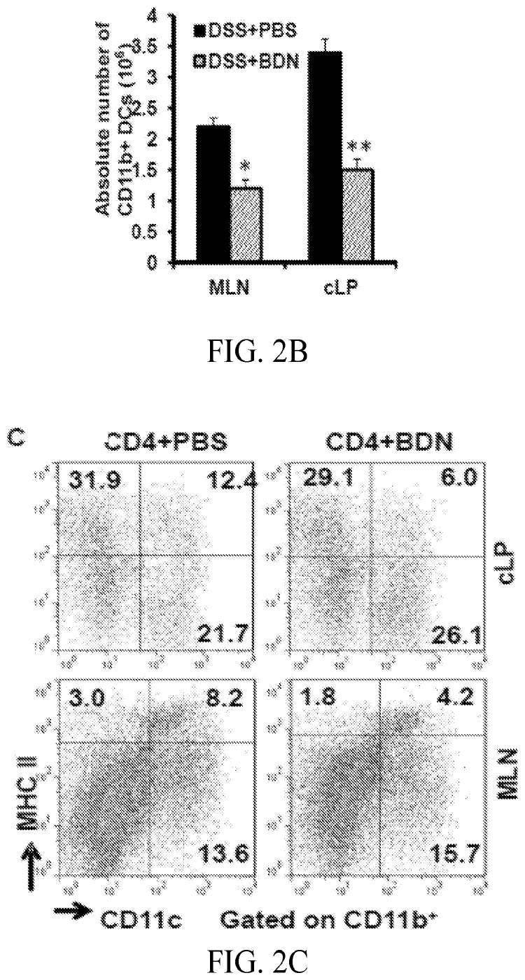

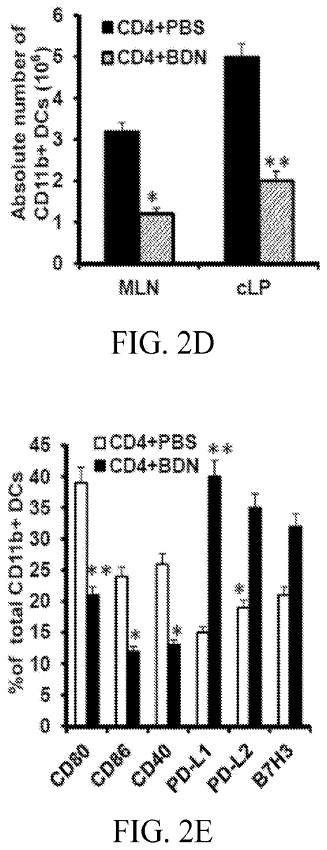

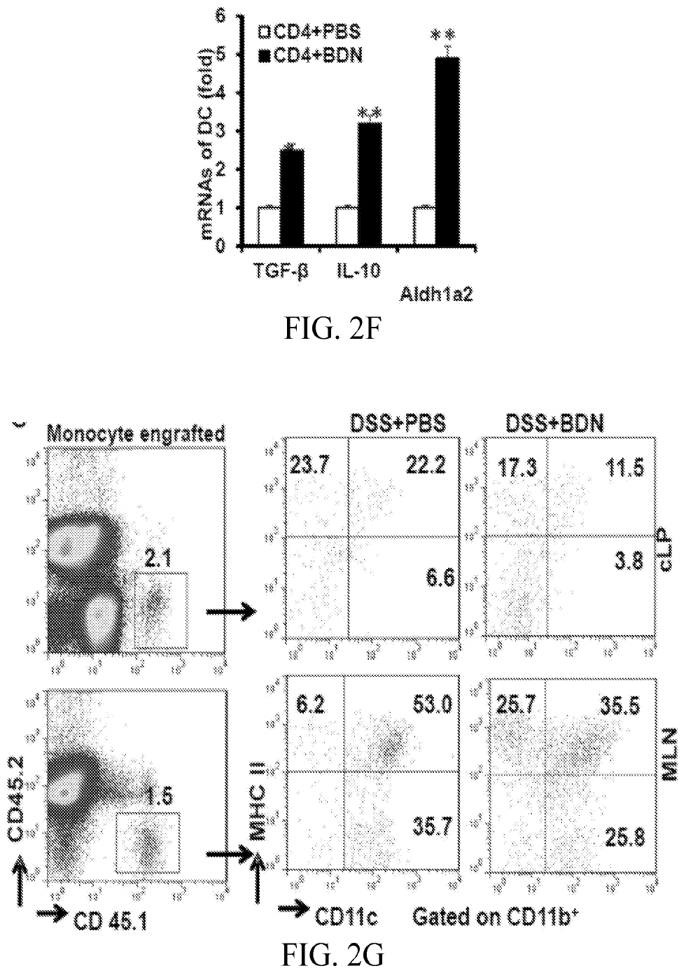

To further identify the cells targeted by BDNs, mice with colitis were orally administered PKH26 labeled BDNs and BDN presence at a cellular level was determined. It was found the BDNs were taken up by DCs in mouse MLNs and colon in experimental colitis (FIG. 11), providing the rationale for studying the effect of BDNs on DCs. In DSS-induced colitis, mice treated with PBS had a more than 2-fold increase in the frequency of CD11c⁺MHCII⁺CD11b⁺ DCs (CD11b⁺ DCs) in the colon and MLNs compared to BDN treated mice (FIG. 2A). The absolute number of CD11b⁺ DCs isolated from the inflamed MLNs and colon of BDN-treated mice was reduced significantly as compared with that from the PBS-treated mice (FIG. 2B).

In the T-cell transfer model of colitis, transfer of naïve T cells also resulted in a large accumulation of activated CD11b⁺ DCs in the MLNs and colon (FIG. 2C). Treatment with BDNs significantly decreased the frequency and the number of CD11b⁺ DCs compared to the treatment of PBS (FIGS. 2C-2D). Flow cytometric phenotypic analysis further revealed that BDNs inhibited the expression of the DC maturation marker CD86, CD80, and CD40, but upregulated the expression of the T cell inhibitory molecule PD-L1, as well as PD-L2 and B7H3 (FIG. 2E), which are molecules associated with T regulatory cells and oral tolerance development. To gain insight into the CD11b⁺ DC genes induced as a result of BDN treatment, gene expression profiling was performed by qPCR. It was found that BDN-derived CD11b⁺ DCs expressed higher amounts of TGF-β, IL-10, or Aldh1a2 (FIG. 2F), which are genes associated with a tolerogenic DC signature. Collectively, these data indicated that BDNs inhibit activation of intestinal CD11b⁺ DCs.

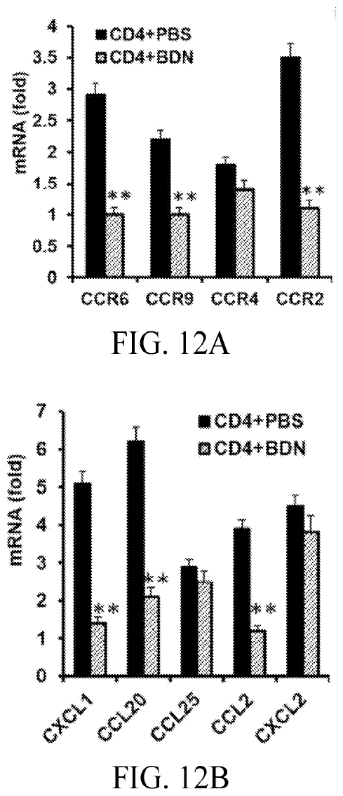

It is also appreciated that chemoattractant molecules play a role in regulating migration of monocytes to sites of inflammation and that CCR6 expression marks a subset of inflammatory DCs within the small intestine. Next, it was determined whether BDNs also inhibit the recruitment of monocytes into inflamed colon by preventing the induction of chemotactic chemokines. It was found that BDN treatment strongly decreased CCR2, CCR9 and CCR6 mRNA expression in CD11b⁺ DCs when compared to PBS treatment (FIG. 12A). To further assess whether the reduced CD11b⁺ DC infiltration after BDN administration is caused by an impaired migration of monocytes, experiments evaluated CXCL1, CXCL2, CCL2, CCL25 and CCL20, which are used by monocytes to migrate into inflamed colon tissue of mice. It was found that the levels of CCL2, CXCL1 and CCL20, but not the levels of CCL25 and CXCL2, were significantly lower in BDN-treated mice compared with PBS-treated Rag1^{-/-} mice on day 35 after induction of colitis (FIG. 12B). Since bone marrow (BM)-derived Gr-1⁺CCR2⁺CX₃CR1^{lo}CD115⁺ monocytes home to sites of inflammation where they further differentiate into inflammatory DCs, it was then determined whether BDNs also inhibited the differentiation of Gr1⁺ monocytes into inflammatory CD11b⁺ DCs in vivo using adoptive transfer experiments. Flow cytometry-sorted Gr-1⁺ BM monocytes from CD45.1 mice were injected i.v. into colitic CD45.2 recipients, which were administered BDNs or PBS. After 72 hr, CD45.1 cells present in MLNs and colon were examined for the differentiation of Gr1⁺ monocytes into CD11b⁺ DCs. Upon transfer into colitic mice that did not receive BDNs, the initial CD45.1⁺Gr-1⁺ BM monocytes mostly differentiated into CD45.1⁺CD11b⁺CD11c⁺MHCII⁺ cells. In contrast, upon transfer into colitic mice receiving BDNs, CD45.1⁺Gr-1⁺ BM monocytes failed to differentiate into CD45.1⁺CD11b⁺ DCs (FIG. 2G). These data indicated that during colitis, BDNs inhibit the activation of DCs as well as the recruitment of monocytes into inflamed colon by preventing the induction of chemotactic chemokines.

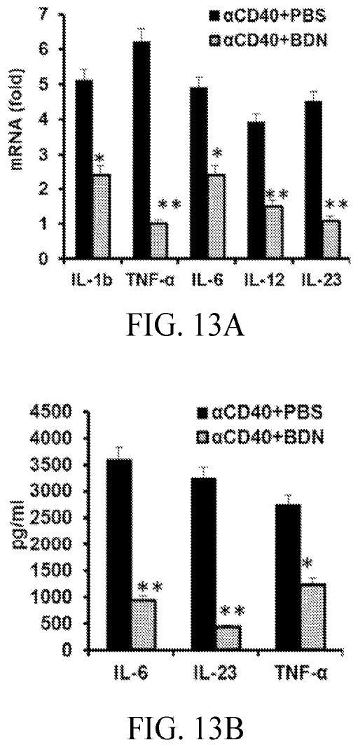

Next, it was determined whether preventing the activation of dendritic cells by BDNs was essential for inhibiting mouse colitis. To test this question, an agonistic αCD40 antibody (FGK45) was used to induce colitis in Rag1^{-/-} mice. Due to the absence of T and B lymphocytes, Rag1^{-/-} mice develop acute colitis within 1 week after □CD40 administration that is driven primarily by macrophages and DCs. Following injection of anti-CD40, PBS-treated Rag1^{-/-} mice rapidly lost weight and developed colitis as previously described. In comparison to PBS-treated Rag1^{-/-} mice, BDN treated Rag1^{-/-} mice developed considerably milder colitis as judged by weight loss, histological scores and their disease activity index (DAI) (FIGS. 3A-3C). FACS analysis revealed BDNs significantly inhibited the accumulation CD11b⁺ DCs in MLNs and LPLs isolated from the small intestine 36 hr after injection of anti-CD40 (FIG. 3D). Real-time PCR results confirmed that amounts of TNF-α, IL-6, IL-23 and IL-12 mRNA in colon were decreased in BDN-treated mice compared with PBS-treated mice (FIG. 13A). ELISA analysis results also showed that cytokines IL-6, IL-23, and TNF-α were reduced after oral treatment with BDNs (FIG. 13B). Immunohistochemistry staining of the colon tissues with monoclonal antibodies directed against the markers F4/80 and CD11b/CD11c indicated a reduced infiltration of CD11b+ DCs in BDN treated Rag1−/− mice when compared to Rag1−/− mice after □CD40 induction (FIG. 3E). However, no significant infiltration of CD11b+F4/80+ macrophages was observed in BDN treated Rag1−/− mice when compared to Rag1−/− mice after □CD40 induction (FIG. 3E). Collectively, these data indicate that preventing activation of DCs is one of the cellular mechanisms underlying BDN mediated prevention of mouse colitis.

### Example 3-BDN-Derived Lipid Induces Tolerogenic DCs

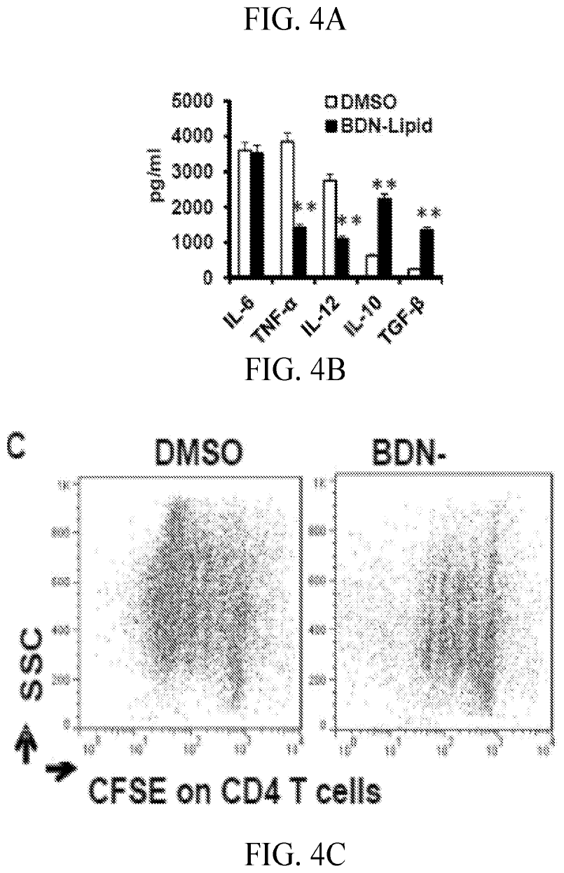

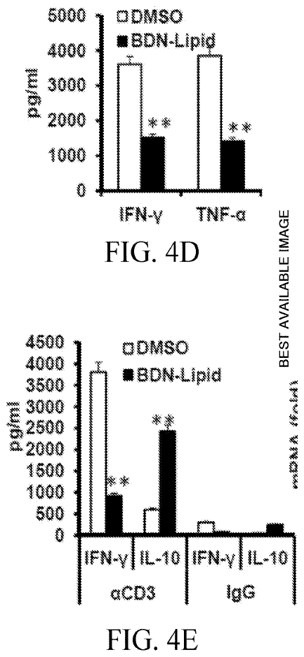

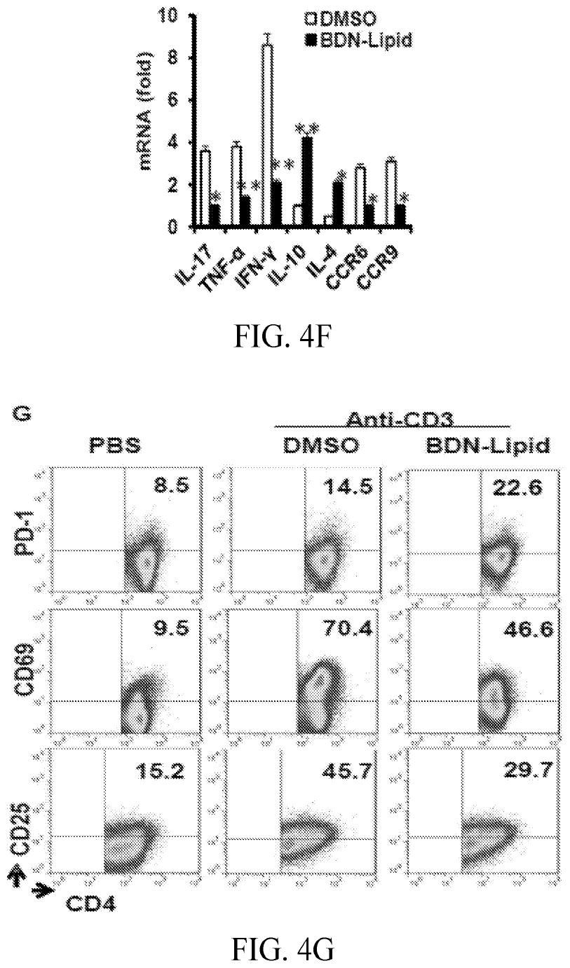

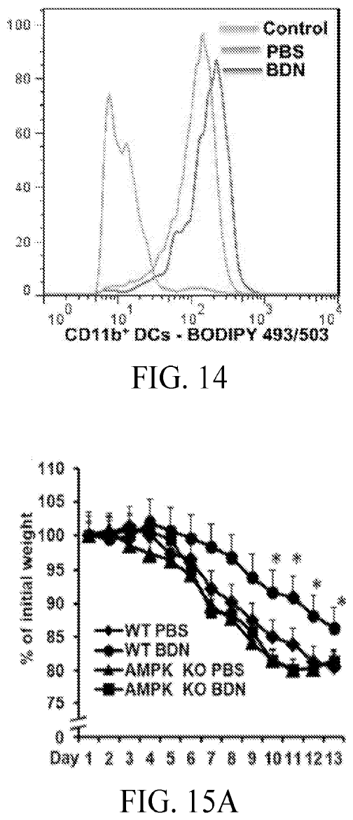

It was next tested whether BDN-derived lipid has a role in inducing DC tolerance. First, the lipophilic fluorescent dye BODIPY 493/503 was used to determine whether the amount of lipids in DCs is increased after mice were fed with BDNs. CD11b⁺ DCs isolated from the colon of colitic mice receiving BDNs showed high levels of lipid in comparison to mice receiving PBS (FIG. 14). To investigate whether BDN-derived lipid has a direct regulatory effect on DCs, the effect of BDN lipid treatment on in vitro-generated bone marrow (BM) DCs was examined. BMDCs were stimulated with LPS in vitro in the presence or absence of BDN-lipid. Exposure to BDN-derived lipid impaired the ability of DCs to respond to LPS, as BDN-lipid conditioned BMDCs exhibited down-regulation of surface expression of MHC class II and the co-stimulatory molecules CD80 and CD86 when compared with unconditioned BMDCs (FIG. 4A). Furthermore, stimulation of DCs with LPS for 24 hours led to a significantly reduced induction of IL-12 and TNF-α in BDN-treated DCs compared with control DCs. BDNs preferentially induced the expression of IL-10 and TGF-β but not IL-6 in DCs (FIG. 4B). These findings demonstrated that BDN-lipid treatment renders BMDCs non-responsive to endotoxins such as LPS. The establishment of tolerance to food antigens relies on the ability of APCs to migrate through the lymphatics to the draining lymph nodes. The mucosal immune system constitutively surveys the intestinal environment and DCs sample intestinal contents to coordinate T cell reactions. Finally, to determine the impact of BDN-lipid on the ability of DCs to present bacteria-associated antigens, lamina propria (LP) DCs (LP-DCs) were isolated and activated with bacterial antigens isolated from the cecum (CBA) in the presence or absence of BDN-lipid. LP-DCs were loaded with ovalbumin (OVA) and then co-cultured with antigen-specific, carboxyfluorescein succinimidyl ester (CFSE)-loaded OT-II naïve T cells for 4 days. OT-II T-cell priming and proliferation were evaluated by flow cytometry analysis in terms of CFSE dilution. As shown in FIGS. 4C-4D, a significant decrease in the proliferation of CD4+ T cells, as well as a decrease in the production of TNF-α and IFN-γ, was observed in cultures supplemented with BDN-lipid. IL-10 production is thought to prevent colitis by suppressing unwanted inflammation, and thus, it was further examined if BDNs promote anti-inflammatory immune responses in BMDC-T cell co-cultures. BMDCs were treated with BDNs, washed, and cultured with naïve CD4⁺ T cells. BDNs induced the expression of IL-10 during in vitro DC-T cell co-culture, while minimal IL-10 was detected from DCs alone (FIG. 4E). Addition of anti-CD3 antibody to the co-culture greatly increased IL-10 production, indicating the main source of IL-10 was from CD4⁺ T cells (FIG. 4E). The results of real-time PCR showed that CD4⁺ T cells co-cultured with BDN-lipid-treated DCs contained lower levels of IL-17A, IFN-γ, IL-6, the T-cell homing markers CCR6 and CCR9, and higher levels of IL-10 and IL-4 than did CD4⁺ T cells co-cultured with PBS (FIG. 4F). FACS analysis showed that CD4⁺ T cells co-cultured with BDN-lipid-treated DCs had a lower expression of T cell activation markers CD69 and CD25 than did CD4⁺ T cells co-cultured with PBS, but higher levels of the negative costimulatory molecules PD-1 (FIG. 4G). Collectively, these data underscored the idea that BDN lipid was driving the induction of CD11c⁺ tolerogenic DCs, which favors the induction of IL-10⁺ and PD-1⁺ T cells for inhibition of gut inflammation.

### Example 4—BDN Lipid Mediated Activation of DC AMPK Plays a Role in Protection of Mice Against Mouse Colitis

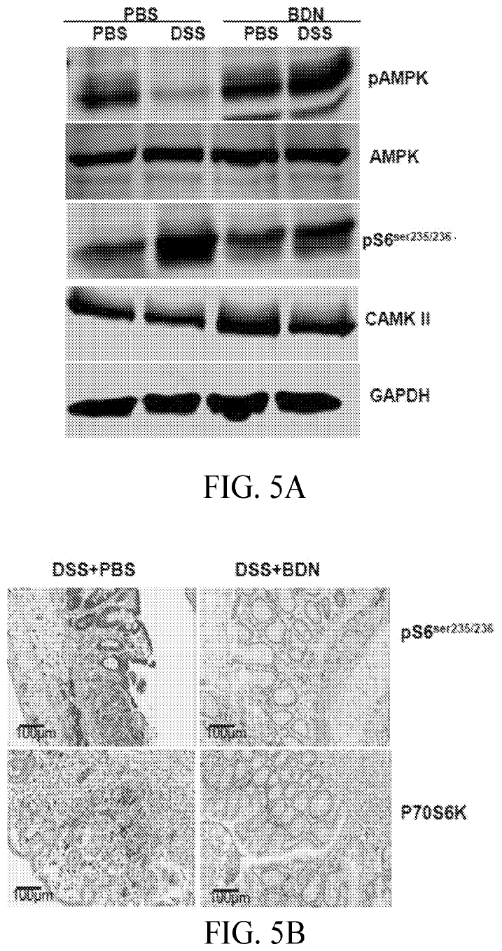

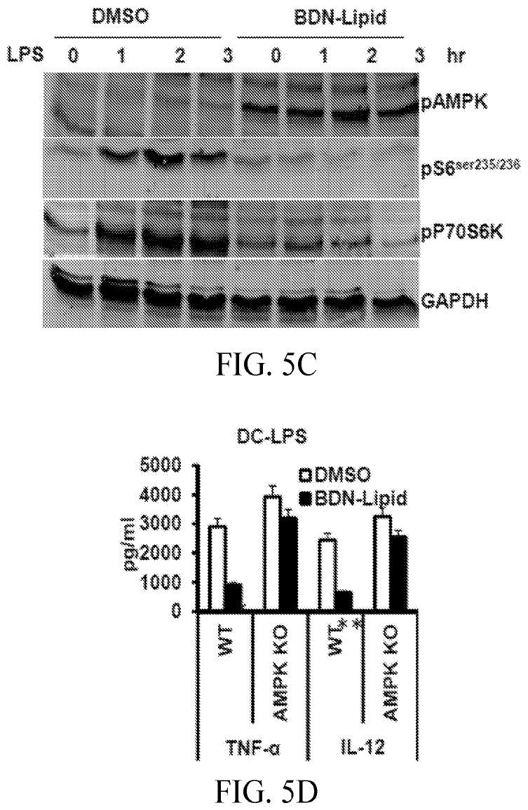

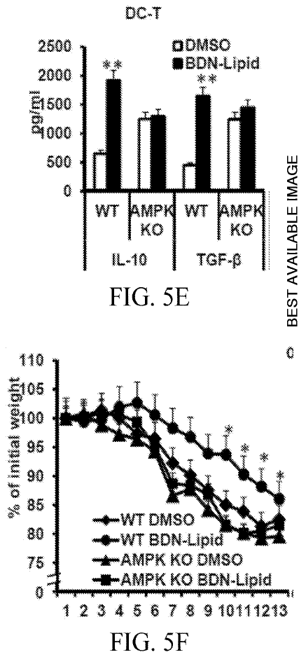

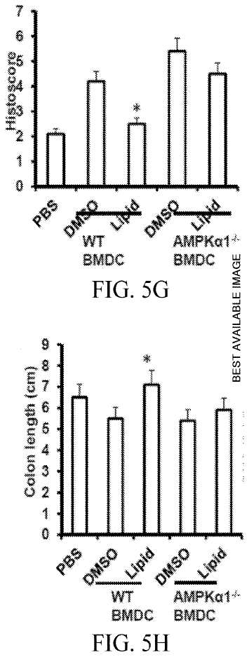

AMPK has an anti-inflammatory role. DCs generated from AMPKα1-deficient mice produce higher levels of proinflammatory cytokines and decrease production of the anti-inflammatory cytokine IL-10 in response to TLR and CD40 stimulation To decipher the molecular mechanisms that underlie BDN mediated inhibition of colitis development, it was investigated whether BDNs mediate its anti-inflammatory effects through AMPK signaling. Western blotting (WB) and confocal immune staining to detect AMPK phosphorylation revealed a higher level of activated AMPK protein in colonic tissues of BDN-treated mice compared with that in PBS-treated mice (FIG. 5A). AMPK has been reported to regulate mTOR/S6K. Western blotting of colon extracts confirmed lower levels of phosphorylated S6 kinase (S6K), a target for PI3K-mTOR signaling, in the colons of BDN-treated mice (FIG. 5A). Immunohistochemical staining further confirmed that treatment with BDNs inhibited mTOR signaling, as evidenced by decreased phosphorylation of S6 and p70S6K compared with the treatment of PBS (FIG. 5B). Collectively, these findings indicated that BDNs enhance AMPK signaling and might promote induction of AMPK-activated anti-inflammatory factors.

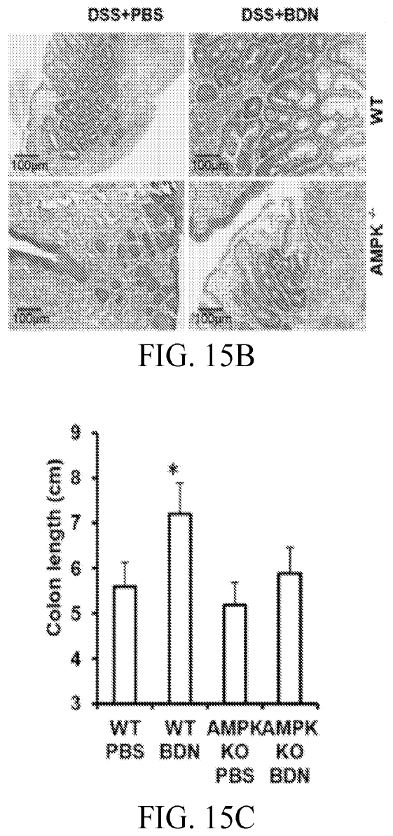

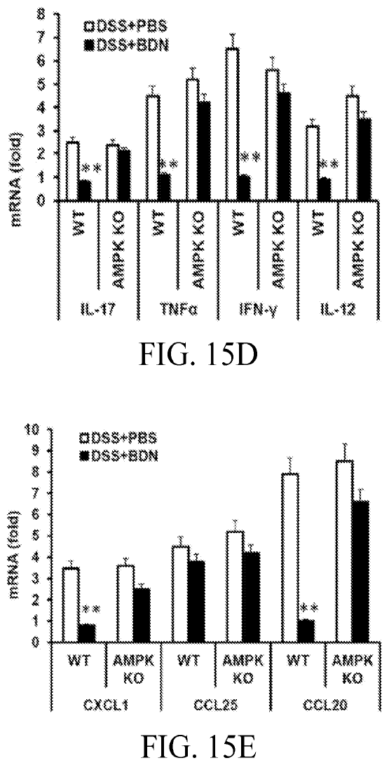

Next, it was investigated whether BDN-derived lipid mediated activation of AMPK has a role in induction of tolerogenic DCs. BDN-derived lipid induced the activation of AMPK and reduced the phosphorylation of p70S6K and S6 in BMDCs (FIG. 5C). Moreover, BDNs inhibited the secretion of TNF-α and IL-12; whereas, the ability to inhibit the secretion of TNF-α and IL-12 from AMPX-deficient DCs was lost in response to LPS stimulation (FIG. 5D). Furthermore, wild-type and AMPK^{-/-} DCs were treated with BDN-lipid and subsequently co-cultured the DCs with CD4⁺ T cells. Compared to wild-type DCs, the absence of AMPK led to an almost complete loss in the production of IL-10 and TGF-β in response to BDN-lipid in DC-T cell co-cultures (FIG. 5E). Collectively, these results showed that BDN-derived lipid has a direct effect on the induction of tolerogenic DCs via the enhancing of AMPK activity. Finally, it was tested whether AMPK was required for BDN-mediated protection from mouse colitis. The effect of BDN on DSS colitis was first tested in AMPKα1^{-/-} animals. While BDNs restored the body weight, colon damage and length (FIGS. 15A-15C), and suppressed the production of pro-inflammatory cytokine and chemokines (FIGS. 15D-15E) in AMPK wild-type littermate controls, AMPKα1^{-/-} mice were not protected (FIGS. 15A-15E). To demonstrate whether DC AMPK was required for BDN lipid mediated induction of tolerogenic DCs, BDN-lipid-treated BMDCs were transferred into mice with DSS colitis. BDN-lipid-treated WT DCs protected animals from disease compared to control (DMSO) treated cells (FIG. 5F). However, BDN-lipid-treated AMPKα1^{-/-} DCs failed to protect animals from disease (FIG. 5F), demonstrating that DC AMPK was required for BDN-lipid-mediated protection. Accordingly, less colon tissue damage and less reduction in colon length were evidenced in mice that received BDN-lipid-treated WT DCs, but not AMPK-deficient DCs (FIGS. 5G-5H. Pro-inflammatory molecules were also analyzed within intestinal tissues of DSS induced colitis following DC transfer. The cytokines IFN-γ, IL-17A, and TNF-α were reduced in the colon following transfer of WT DCs incubated with BDN-lipid (FIG. 5I). Transfer of BDN-lipid-treated AMPKα1^{-/-} DCs into animals resulted in cytokine production as high as that detected in untreated AMPKα1^{-/-} DCs (FIG. 5I). Collectively, these findings showed that AMPK was a factor required for BDN-lipid mediated induction of tolerogenic DCs.

### Example 5—BDN Sulforaphane (SFN) Induces Tolerogenic DCs Via AMPK Signaling

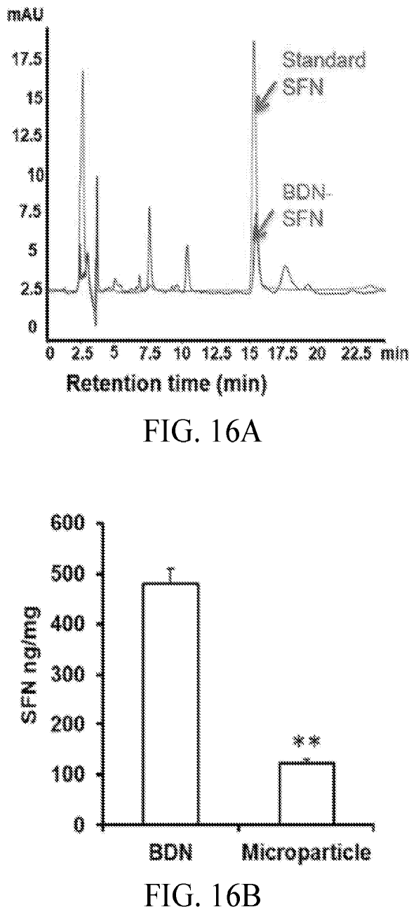

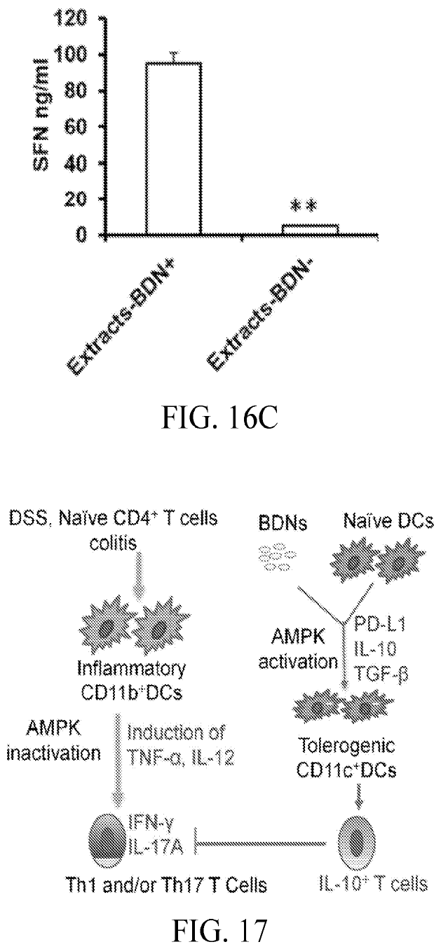

Experiments were further undertaken to identify which BDN lipid(s) contributes to induction of tolerogenic DCs. It is appreciated that SFN is related to the anti-inflammatory effects of broccoli. To date, however, most of the data have been derived from using SFN enriched broccoli extracts. The biological effect of SFN in the context of broccoli has not been investigated. HPLC analysis of BDN lipids indicated that SFN was enriched in nanoparticles compared to that in microparticles isolated from broccoli extracts and very little SFN was present in free form in broccoli extracts (FIG. 16).

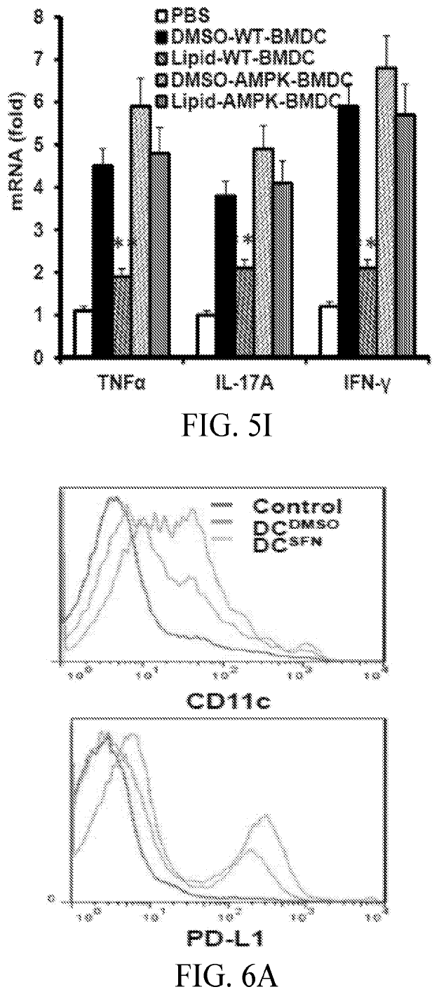

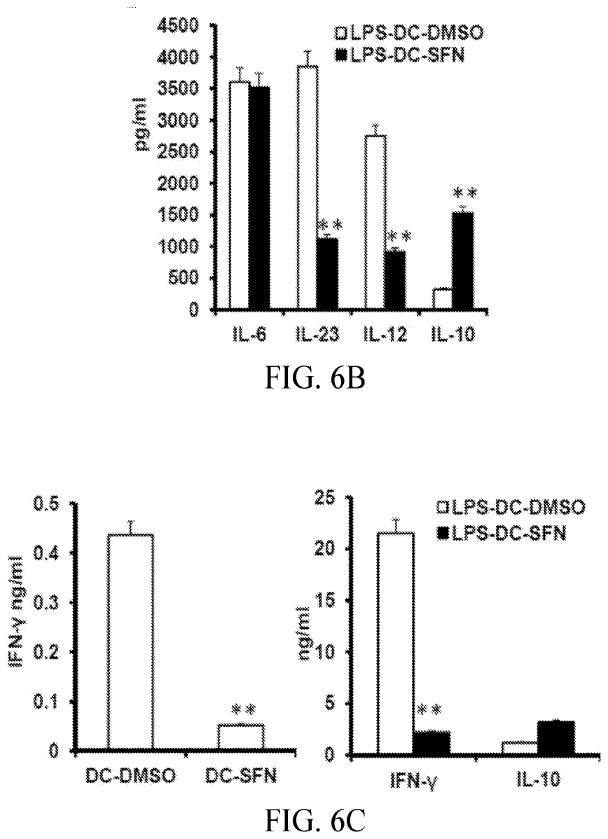

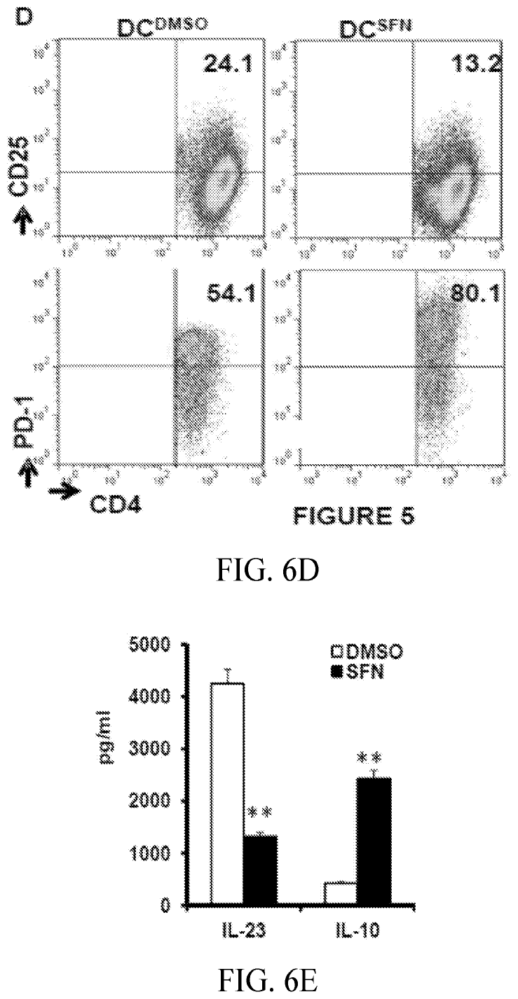

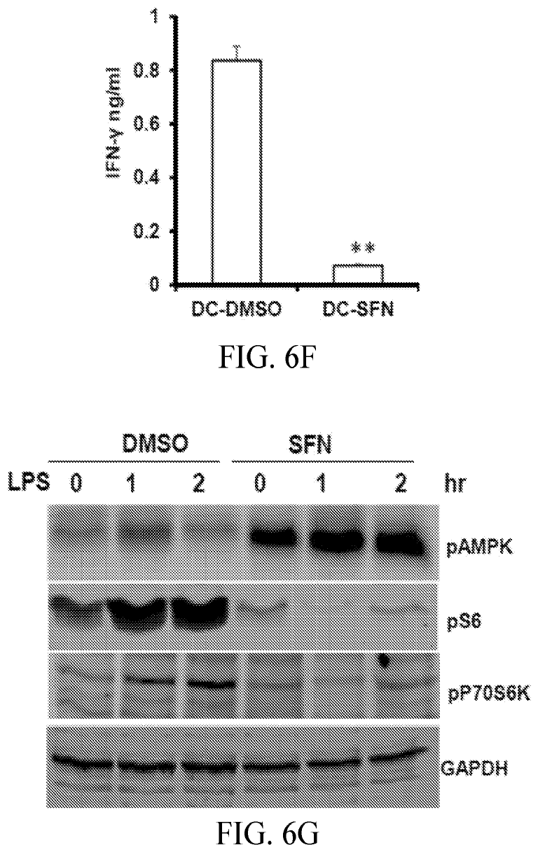

Monocyte-derived immature DC differentiated for 7 d with granulocyte-macrophage colony-stimulating factor (GM-CSF) and IL-4 in the absence or presence of SFN were next assessed. Addition of SFN and GM-CSF led to the differentiation of bone marrow cells with a lower expression of CD11c and a higher expression of PD-L1 (a marker associated with regulatory DCs) than addition of GM-CSF alone (FIG. 6A). The effect of SFN on LPS stimulated DC activation was then assessed. Although DCs stimulated with LPS showed similar upregulation of maturation markers in the presence or absence of SFN, SFN-exposed LPS-stimulated DCs (LPS-DC^{SFN}) produced less IL-12, IL-23 and more IL-10 than did DCs matured with LPS alone (LPS-DC^{DMSO}) (FIG. 6B).

To further examine the effect of SFN treated DCs on priming T cells, SFN-treated DCs (DC^{SFN}) or control DCs (DC^{DMSO}) were cultured together with CD4 T cells. Priming with DC^{SFN} resulted in negligible production of IFN-γ compared with that of DC^{DMSO} (FIG. 6C, left). Furthermore, CD4⁺ T cells primed with LPS-DC^{SFN} also produced less IFN-γ and more IL-10 than did CD4⁺ T cells primed with LPS-treated DC^{DMSO} (FIG. 6C, right). Moreover, DC^{SFN} induced CD4⁺ T cells expressed higher levels of the negative costimulatory molecules PD-1 and a lower expression of T cell activation marker CD25 than did DC^{DMSO} (FIG. 6D). Thus, SFN impaired the activation and stimulatory capacity of monocyte-derived DCs. Using identical experimental approaches except that an alloreactive CD4 T cell assay was employed, a similar inhibitory effect of SFN treated human monocyte derived DCs on priming alloreactive CD4 T cells was observed (FIGS. 6E-6F). These studies were extended to address whether SFN-derived DCs have an enhanced immunoregulatory potential by activating AMPK. To this end, human monocyte-derived DCs were stimulated with SFN and/or with LPS. LPS stimulation resulted in a significant reduction of AMPK phosphorylation; however, the treatment of SFN increased phosphorylation of AMPK at Thr172, which reflects AMPK activity (FIG. 6G). Enhanced AMPK phosphorylation was paralleled by decreased S6 phosphorylation and another well-characterized mTORC1 target, p70S6K (FIG. 6G). These results indicated that SFN activates human DC AMPK. Moreover, these data generated from human monocyte-derived DCs could be applied to the clinical setting, particularly human tissue transplantation.

### Example 6—Sulforaphane-Enriched BDNs Ameliorate Murine Colitis by Inducing Regulatory DCs

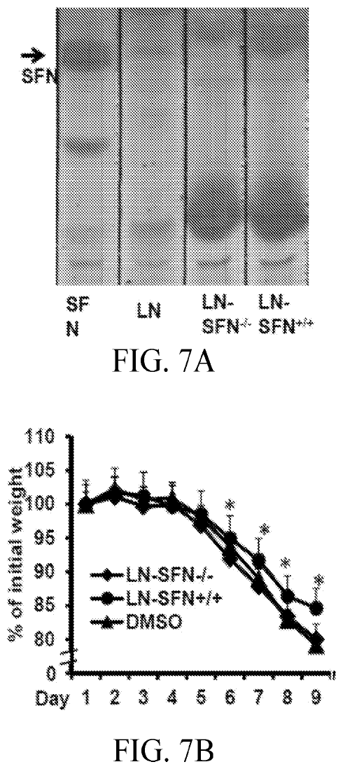

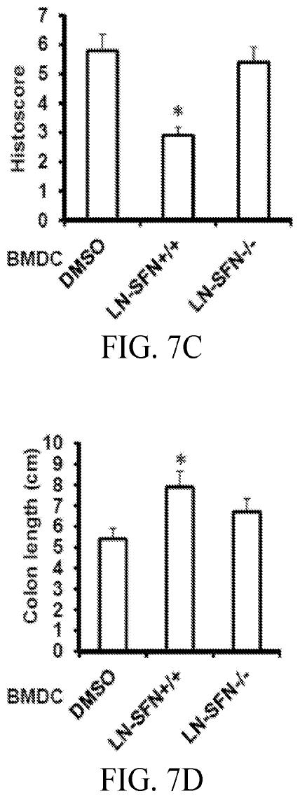

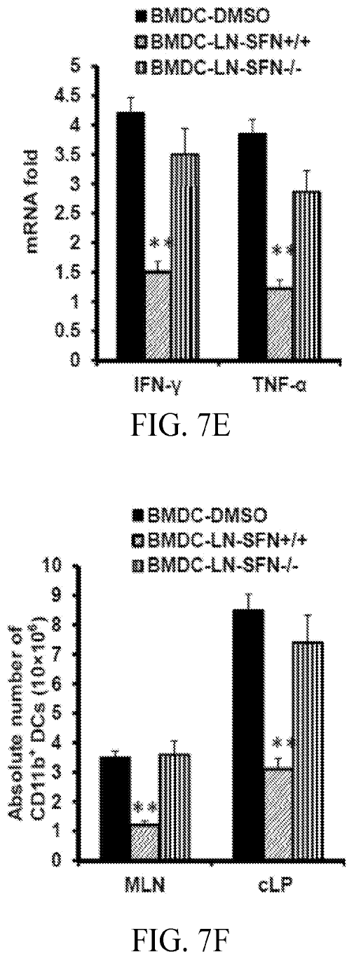

The capability of SFN to induce tolerogenic DCs in vitro prompted examination of the in vivo tolerogenic effect of BDN associated SFN in DSS-induced colitis. Using knock out and knock in strategies, it was next determined whether BDN associated SFN plays a role in DSS-induced colitis in the context of lipids extracted from BDNs. For knockout, lipids of BDNs without SFN were carefully recovered from TLC silica gel plates, then liposome-like nanoparticles (LN) with SFN knock-out (LN^{SFN-/-}) were generated using previously described technology. For knock in, commercial SFN was added to SFN knock-out lipid to make knock-in liposome-like nanoparticles (LN^{SFN+/+}) (FIG. 7A).

The effects) of knock-out of SFN was further evaluated in terms of inhibition of DSS-induced colitis. Adaptively transferring LN^{SFN+/+}-treated DCs to mice protects animals from disease compared to LN^{SFN-/-} treated DCs (FIG. 7B). Collectively, these results demonstrated that BDN lipids play a role in BDN-mediated protection from DSS induced colitis and BDN SFN contributes to BDN-mediated protection. This conclusion was also supported by the results showing that less colon tissue damage and less reduction in colon length were evidenced in mice adoptively receiving LN^{SFN+/+} treated DCs compared to mice receiving LN^{SFN-/-} treated DCs (FIGS. 7C-7D). A reduction of the cytokines IFN-γ, IL-17A, and TNF-α in the colon following transfer of LN^{SFN-/-} treated DCs compared to LN^{SFN+/+} treated DCs (FIG. 7E) also supports this conclusion. Moreover, lower numbers of BMDC^{SFN} reached the MLNs and colon of treated mice (FIG. 7F), which suggests that SFN has an effect on the in vivo migratory ability of DCs. Collectively, these data support the idea that BDN SFN contributes to BDN-mediated protection from mouse colitis.

### Discussion of Examples 1-7

Whether nanoparticles present in edible plants have a direct beneficial effect on human health has not been proven. It was hypothesized that edible nanoparticles regulate intestinal immune homeostasis by targeting DCs. In the foregoing studies, broccoli-derived nanoparticles (BDN) were used. The data indicated that BDNs mediating activation of DC AMPK is one mechanism underlying BDN induced tolerogenic DCs (FIG. 17). Furthermore, as suggested by the data from AMPK knockout mice, the mechanisms by which BDN-derived SFN affects homeostasis or the functions) of DCs are demonstrated in these studies.

Moreover, the biological effects of edible nanoparticles on gut immune homeostasis could be further amplified through induction of other immune cells induced by edible nanoparticle educated DCs, such as induction of CD4⁺IL-10⁺ T cells as was demonstrated in these studies (FIG. 2F and FIG. 4F). Unlike the free form of SFN which cannot be target-delivered to DCs, the above-described findings highlight that SFN carried by BDNs is delivered to LP DCs and then induces tolerogenic DCs. This finding provides a new avenue for not only further studying how SFN leads to DC AMPK activation in vivo, but also identifying additional BDN-derived factors housed in the same compartment (edible nanoparticles) with SFN that may also regulate the AMPK activity of DCs. The above-described findings on the mechanism of action of BDN SFN have implications for other known edible nanoparticles such as nanoparticles from grape, grapefruit, ginger, and tomatoes. Like BDN SFN, nanoparticles from these edible plants may carry functionally similar molecules that induce gut immune tolerance.

Induction and maintenance of tolerogenic DCs is important for oral food tolerance. Although there is evidence that DCs play an important role in the induction of tolerance to soluble antigens, how inert particles like edible nanoparticles gain access to these DCs through the supposedly impermeable epithelial barrier remains a mystery. In the above study, it was shows that BDNs can be taken up efficiently by CD11c⁺ DCs that are capable of inducing tolerance in recipient mice. This finding provides a foundation for further studying whether edible nanoparticles that present food to the gut may carry plant species-specific antigens for induction of food specific oral tolerance. An improved understanding of the interplay among food we eat, DCs, and luminal signals may thus provide new therapeutic strategies for the treatment and/or prevention of diseases due to dysregulation of gut immune homeostasis.

It is appreciated that intestinal DCs can be differentiated and matured in to tolerogenic or immunogenic DCs depending on the stimuli they receive. Daily, people eat a variety of vegetables and fruits all of which contain nanoparticles. In the above study, although it was show that BDN is DC tolerogenic, it was speculated that other edible nanoparticles could be DC immunogenic. It was conceivable that eating both tolerogenic and immunogenic edible nanoparticles presented in the food will be beneficial for maintaining gut immune homeostasis (Yin-Yang balance). Therefore, the finding in the above-described study established a foundation for establishing edible nanoparticle profiles and further classifying them based on their immune regulatory role.

### Materials and Methods for Examples 8-12

Animals and treatments. The CSN8-floxed mouse model (CSN8^{flox/flox}) was originally created as described previously. To delete CSN8 in IECs, Villin-Cre⁺/CSN8^{flox/flox} mice (termed CSN8^{ΔIEC}) were generated by crossing CSN8^{flox/flox} mice with Villin-Cre transgenic mice. The Villin-Cre mice were in C57BL/6 background. Littermate Villin-Cre⁻/CSN8^{flox/flox} mice (termed CSN8^{ΔIEC}) were obtained and used as control. The CSN8^{flox/flox}/Villin-Cre⁺ animals were further bred with APC^{Min/+} mice (Jackson Laboratory, Bar Harbor, Me.) to generate APC^{Min/+}Villin-cre⁺CSN8^{flox/flox} mice (termed APC^{Min/+}CSN8^{ΔIEC}). Littermate APC^{Min/+}Villin-Cre⁻ CSN8^{flox/flox} mice, APC^{Min/+}Villin-Cre⁺CSN8^{+/+}mice, or APC^{Min/+}Villin-Cre⁺CSN8^{flox/+} mice were used as control (termed APC^{Min/+}CSN8^{fl/fl}). All animal procedures were approved by the University of Louisville Institutional Animal Care and Use Committee. For antibiotic treatment, a combination of ampicillin (1 g/L), gentamicin (1 g/L), vancomycin (500 mg/L), neomycin sulfate (1 g/L), and metronidazole (1 g/L) was added to drinking water. For fecal transfer experiments, Fecal pellets from APC^{Min/+}CSN8^{fl/fl} and APC^{Min/+}CSN8^{ΔIEC} mice were suspended in 1 ml of phosphate-buffered saline (PBS). The suspension from each fecal pellet was used for oral gavage (100 μl) of two recipient mice once a week for four weeks. For BDNs treatment, 8-week-old APC^{Min/+}CSN8^{fl/fl} and APC^{Min/+}CSN8^{ΔIEC} mice were given BDNs orally (250 μg/mouse in PBS) twice every week for 14 weeks. The University of Louisville IACUC approved all animal procedures.

Chemically induced colitis models. Colitis was induced in 8-week-old CSN8^{fl/fl} or CSN8^{ΔIEC} mice by the addition of 2% (wt/vol) DSS (36-50 KD molecular weight, MP Biomedicals, OH) in their drinking water. To induce colorectal tumors, a combination of the carcinogen AOM with repeated administration of DSS in the drinking water was used. Mice (8-10 weeks old) were injected intraperitoneally with a single dose of AOM (10 mg/kg; Sigma, # A2853). After 5 days, 2% DSS was given in the drinking water for 5 days, followed by 14 days of regular drinking water. The DSS treatment was repeated for two additional cycles, and mice were sacrificed 90-100 days after the AOM injection. Body weights, stool consistency, and GI bleeding were recorded during DSS treatment. Colons were collected immediately after sacrifice and fixed as “swiss-rolls” in 10% formalin solution at room temperature overnight, and paraffin embedded. Clinical scores and colonic damage scores were estimated as detailed previously. Tumor size measurements were performed using a digital caliper in a blinded fashion.

Disease monitoring and scoring. Mice were weighed daily and monitored for appearance and signs of soft stool and diarrhea. A combinatorial index of disease, or disease activity index (DAI), defined as stool blood, stool form, and weight loss was used to analyze the degree of colitis. Histologic grades and inflammation was assessed with a modified scoring system. Each sample was graded semi-quantitatively from 0 to 3 for the four following criteria: degree of goblet cell depletion and epithelial hyperplasia; leukocyte infiltration in the lamina propria; area of tissue affected and the presence of markers of severe inflammation such as crypt abscesses, submucosal inflammation, and ulcers. Scores for each criterion were added to give an overall inflammation score for each sample of 0-8.

Reagents, antibodies and flow cytometry. Sulforaphane was purchased from Sigma-Aldrich. For analysis of surface markers, cells were stained in PBS containing 2% (wt/vol) BSA. Intracellular staining of the transcription factors Foxp3 was performed using the Foxp3 Fix/Perm Buffer Set (eBioscience). For detection of intracellular cytokines, cells were first stimulated for 4 h with 50 ng/ml PMA (phorbol 12-myristate 13-acetate, Sigma) and 1 μg/ml ionomycin (Sigma) in the presence of Brefeldin A (5 μg/ml; All obtained from Sigma), followed by staining for surface markers. Cells were then fixed and permeabilized using the Foxp3 Fix/Perm Buffer Set (eBioscience) and stained for intracellular cytokines. Cells were subjected to FACS analysis with the following antibodies at a dilution of 1/200-1/600: PE- or APC-labeled anti-IL-4 (11B11, eBioscience), PE-labeled anti-Foxp3 (FJK-16s, eBioscience), PerCP-Cy5.5, PE-, FITC- or APC-labeled anti-IL-17A (TC11-18H10.1), PE- or APC-labelled anti-IL-10 (JES5-16E3), APC- or PE-Cy7-labeled anti-IFN (XMG1.2), PE-, FITC- or APC-labeled anti-CD11b (M1/70), PE-, FITC- or APC-labeled anti-CD4 (RM4-5), PE-Cy7-labeled anti-CD3 (145-2C11), PE-anti-Gr-1 (RB6-8C5), PE- or FITC-labeled anti-mouse Ly6G (1A8), APC-conjugated CD45.2 (104), PE-conjugated anti-CD45.1 (A20), FITC-, PerCP-Cy5.5 or Pacific Blue-labelled anti-CD45 (30-F11), PE-anti-CCR4 (2G12), PE- or FITC-labelled anti-CCR9 (CW-1.2). All antibodies were obtained from Biolegend unless otherwise noted. Flow cytometry data were acquired on a 5-color FACScan (Becton Dickinson) and analyzed using FlowJo software (Treestar). Cell sorting was performed using a FACSAria II.

Histology and immunohistochemistry. Tissue specimens were fixed in 10% formalin, dehydrated, and then embedded in paraffin. Tissue samples were cut at 5 μm thicknesses and stained with hematoxylin and eosin. For immunofluorescence analysis, tissue sections were subjected to antigen retrieval by boiling the slides in Antigen Unmasking Solution (Vector Laboratories) for 10 minutes according to instructions. Sections were then blocked for 1 hour at 22° C. with 5% BS A in PBS and incubated overnight at 4° C. with the primary antibodies, i.e., rabbit polyclonal Ki67 (1:200, Thermo Scientific), anti-Lysozyme (Abeam), anti-Chromogranin A (Abeam), E-cadherin, anti-cleaved caspase3 and phospho-StaG (SerTyr705) antibody (Cell Signaling) used at a dilution of 1/250, anti-CSN8 (1:200, provided by Dr. Ning Wei), mouse monoclonal anti-SMOX, anti-Brdu (1:200, AbD seroTec), anti-SSAT and ODC (1:100, Santa Cruz Biotechnology). Primary antibodies were detected by Alexa Fluor 488,594 or 647 conjugated goat anti-mouse, anti-rabbit IgG and anti-rat (1:600, Invitrogen). For Alcian blue staining, deparaffinized and rehydrated slides were incubated for 30 min in Alcian blue solution, pH 2.5, and were counterstained with nuclear Fast Red. To monitor intestinal epithelial proliferation and migration, mice were injected intraperitoneally (i.p.) with 50 mg/kg of 5′-bromo-2′-deoxyuridine (BrdU, Sigma Aldrich) in PBS 24 h or 48 h before organ harvest. Tissues were counterstained with DAPI and images were captured on a Zeiss LSM 510 confocal microscope equipped with a digital image analysis system (Pixera). For immunohistochemistry analysis of macrophages and polyamines enzyme, frozen sections were stained with F4/80 (BM8, ebioscience). OCT (Sakura Finetek)-embedded tissue cryosections (9 μm-thick) were also stained with anti-SMOX, or anti-ODC (Santa Cruz Biotechnology) antibody as primary antibody, followed by staining with horseradish peroxidase-conjugated anti-IgG second antibodies. Antigens were then visualized with 3,3′-diaminobenzidine substrate (Vector Laboratories) and scanned using an Aperio Image scope.

RNA extraction and PCR. Total RNA was isolated from the small intestine and colon or tumor tissue using the Qiagen RNeasy RNA isolation Kit and was used to synthesize cDNA. RNA (1 μg) was reverse-transcribed with Superscript III and random primers (Invitrogen). For quantitation of genes of interest, cDNA samples were amplified in a CFX96 Realtime System (Bio-Rad Laboratories, Hercules, Calif., USA) using SYBR Green Master Mix (Invitrogen) and specific primers (Table 2) according to the manufacturer's instructions. Fold changes in mRNA expression between treatments and controls were determined by the 5CT method as described. Results for each sample were normalized to the concentration of GAPDH mRNA measured in the same samples and expressed as fold increase over baseline levels, which are set at a value of 1. Differences between groups were determined using a two-sided Student's t-test and one-way ANOVA. Error bars on plots represent±SEM, unless otherwise noted. All primers were purchased from Eurofins MWG Operon.

TABLE 2
Primers used for Real-time PCR

| Gene name | Forward primer | Reverse primer |
|---|---|---|
| IL-17F | AATTCCAGAACCGCTCCAG | TTGATGCAGCCTGAGTGTCT |
| | (SEQ ID NO: 1) | (SEQ ID NO: 2) |
| GAPDH | AGGTCATCCCAGAGCTGAACG | ACCCTGTTGCTGTAGCCGTAT |
| | (SEQ ID NO: 3) | (SEQ ID NO: 4) |
| β-Actin | ACGGCCAGGTCATCACTATTC | AGGAAGGCTGGAAAAGAGCC |
| | (SEQ ID NO: 5) | (SEQ ID NO: 6) |
| TNF-α | TCTATGGCCCAGACCCTCAC | GACGGCAGAGAGGAGGTTGA |
| | (SEQ ID NO: 7) | (SEQ ID NO: 8) |
| IL-22 | TTGAGGTCGTCCAACTTCCAGCA | AGCCGGACGTCGTGTTGTTA |
| | (SEQ ID NO: 9) | (SEQ ID NO: 10) |
| IL-17A | TTTAACTCCCTTGGCGCAAAA | CTTTCCCTCCGCATTGACAC |
| | (SEQ ID NO: 11) | (SEQ ID NO: 12) |
| CXCL1 | AGCCACCCGCTCGCTTCTCTGTG | AGCCTCGCGATTCTTGAGTGTGG |
| | (SEQ ID NO: 13) | (SEQ ID NO: 14) |
| SDF-1α | CTGTGCCCTTCAGATTGTTG | TCAGCCTTCCTCGGGGGTCT |
| | (SEQ ID NO: 15) | (SEQ ID NO: 16) |
| CCL2 | GTTGGCTCAGCCAGATGCA | AGCCTACTCATTGGGATCATCTTG |
| | (SEQ ID NO: 17) | (SEQ ID NO: 18) |
| IFN-γ | TCAGCAACAGCAAGGCGAAAAAGG | CCACCCCGAATCAGCAGCGA |
| | (SEQ ID NO: 21) | (SEQ ID NO: 22) |
| CCL20 | GTGGGTTTCACAAGACAGATG | TTTTCACCCAGTTCTGCTTTG |
| | (SEQ ID NO: 23) | (SEQ ID NO: 24) |
| CCL25 | GCTTTTTGCCTGCCTGGTTG | TCAGTCTGAGAGTCTGAGGC |
| | (SEQ ID NO: 25) | (SEQ ID NO: 26) |
| IL-10 | TTTGAATTCCCTGGGTGAGAA | GGAGAAATCGATGACAGCGC |
| | (SEQ ID NO: 19) | (SEQ ID NO: 20) |
| IL-6 | GAGAGGAGACTTCACAGAGGATAC | GTACTCCAGAAGACCAGAGG |
| | (SEQ ID NO: 27) | (SEQ ID NO: 28) |
| IL-4 | GAGACTCTTTCGGGCTTTTC | TGATGCTCTTTAGGCTTTCCA |
| | (SEQ ID NO: 29) | (SEQ ID NO: 30) |
| IL-23p19 | GGTGGCTCAGGGAAATGT | GACAGAGCAGGCAGGTACAG |
| | (SEQ ID NO: 31) | (SEQ ID NO: 32) |
| CXCL2 | CGCTGTCAATGCCTGAAG | GGCGTCACACTCAAGCTCT |
| | (SEQ ID NO: 33) | (SEQ ID NO: 34) |
| CXCL10 | CCCACGTGTTGAGATCATTG | TCCATCACAGCACCGGG |
| | (SEQ ID NO: 35) | (SEQ ID NO: 36) |
| IL-12 p35 | TACTAGAGAGACTTCTTCCACAACA | TCTGGTACATCTTCAAGTCCTCAT |
| | AGAG (SEQ ID NO: 37) | AGA (SEQ ID NO: 38) |
| IL-12 p40 | GACCATCACTGTCAAAGAGTTTCTA | AGGAAAGTCTTGTTTTTGAAATTTT |
| | GAT (SEQ ID NO: 39) | TTAA (SEQ ID NO: 40) |
| CCR6 | CCTCACATTCTTAGGACTGGAGC | GGCAATCAGAGCTCTCGGA |
| | (SEQ ID NO: 41) | (SEQ ID NO: 42) |
| CCR9 | CACAGACTTCACAAGCCCTA | GTACAAGGGTGGGAGGAAAT |
| | (SEQ ID NO: 43) | (SEQ ID NO: 44) |
| TGF-β1 | TGACGTCACTGGAGTTGTACGG | GGTTCATGTCATGGATGGTGC |
| | (SEQ ID NO: 45) | (SEQ ID NO: 46) |
| Aldh1a2 | ATGCGGATTGCCAAGGAGG | TGAAGACAGCTGCTACAAGTCC |
| | (SEQ ID NO: 47) | (SEQ ID NO: 48) |
| E. rectale/ | ACTCCTACGGGAGGCAGC | GCTTCTTAGTCAGGTACCGTCAT |
| C. coccoides | (SEQ ID NO: 53) | (SEQ ID NO: 54) |
| group (Erec) | | |
| Segmented | GACGCTGAGGCATGAGAGCAT | GACGGCACGGATTGTTATTCA |
| Filamentous Bacteria | (SEQ ID NO: 55) | (SEQ ID NO: 56) |
| (SFB) | | |
| Lactobacillus/ | AGCAGTAGGGAATCTTCCA | CACCGCTACACATGGAG |
| Enterococcus | (SEQ ID NO: 57) | (SEQ ID NO: 58) |
| group (Lact) | | |
| Mouse Intestinal | CCAGCAGCCGCGGTAATA | CGCATTCCGCATACTTCTC |
| Bacteria (MIB) | (SEQ ID NO: 59) | (SEQ ID NO: 60) |
| Bacteroides (Bact) | GGTTCTGAGAGGAGGTCCC | CTGCCTCCCGTAGGAGT |
| | (SEQ ID NO: 61) | (SEQ ID NO: 62) |
| Eubacteria | ACTCCTACGGGAGGCAGCAGT | ATTACCGCGGCTGCTGGC |
| | (SEQ ID NO: 63) | (SEQ ID NO: 64) |
| Actinobacteria | CGCGGCCTATCAGCTTGTTG | ATTACCGCGGCTGCTGG |
| | (SEQ ID NO: 65) | (SEQ ID NO: 66) |
| Firmicutes | GCTGCTAATACCGCATGATATGTC | CAGACGCGAGTCCATCTCAGA |
| | (SEQ ID NO: 67) | (SEQ ID NO: 68) |
| Proteobacteria | CATGACGTTACCCGCAGAAGAAG | CTCTACGAGACTCAAGCTTGC |
| | (SEQ ID NO: 69) | (SEQ ID NO: 70) |
| Alphaproteobacteria | ACTCCTACGGGAGGCAGCAG | TCTACGRATTTCACCYCTAC |
| | (SEQ ID NO: 71) | (SEQ ID NO: 72) |
| Betaproteobacteria | ACTCCTACGGGAGGCAGCAG | TCACTGCTACACGYG |
| | (SEQ ID NO: 71) | (SEQ ID NO: 73) |
| Epslionproteobacteria | TGGCGSACGGGTGAGTAATRTATAG | GGAGTTTACRCWCCGAAAWGYGT |
| | (SEQ ID NO: 74) | C (SEQ ID NO: 75) |
| Gammaproteobacteria | CMATGCCGCGTGTGTGAA | ACTCCCCAGGCGGTCDACTTA |
| | (SEQ ID NO: 76) | (SEQ ID NO: 77) |
| Bifidobacterium | CGGGTGAGTAATGCGTGACC | TGATAGGACGCGACCCCA |
| | (SEQ ID NO: 78) | (SEQ ID NO: 79) |
| Clostridial cluster | GCACAAGCAGTGGAGT | CTTCCTCCGTTTTGTCAA |
| IV | (SEQ ID NO: 80) | (SEQ ID NO: 81) |
| Bacteroidetes | CATGTGGTTTAATTCGATGAT | AGCTGACGACAACCATGCAG |
| | (SEQ ID NO: 82) | (SEQ ID NO: 83) |

ELISA. The quantity of TNF-α, IL-1β, IL-17A, IL-6, IL-10 and IFN-γ (eBioscience) were determined in culture supernatants, serum and tissue using ELISA kits according to the manufacturer's instructions. The sensitivity of the assay was less than 20 μg/ml.

Western blot analysis. Tissue or cells were disrupted in lysis buffer containing 1% Triton X-100, 0.1% SDS, 150 mM NaCl, 50 mM Tris-HCl, 1 mM EDTA, 1 mM EGTA, 5 mM sodium molybdate and 20 mM phenylphosphate with protease and phosphatase inhibitors (1 mM PMSF, 10 μg ml⁻¹ aprotinin, 20 μg ml⁻¹ leupeptin, 20 μg ml⁻¹ pepstatin A, 50 mM NaF and 1 mM sodium orthovanadate) for 30 min on ice. The samples were centrifuged (16,000 g, 10 min, 4° C.) and the resulting supernatants transferred to fresh tubes. Protein lysates were quantitated using a Bio-Rad protein kit (Bio-Rad) and 50-100 μg of lysates were separated on 10% SDS polyacrylamide gels and transferred to a nitrocellulose membrane. Antibodies against the following proteins were used: Rabbit anti-phospho-StaG (Ser727), phospho-Stat3 (Tyr705), phospho-AKT (ser473), β-Catenin, Cyclin D1, CyclinD2, BAX, c-myc, p21, p27, Caspase-3, Cleaved Caspase-3 or STAT3 and β-actin were purchased from Cell Signaling Technology (Danvers, Mass.) and used at a dilution of 1/1,000. ODC, SMOX, SSAT, Cullin 1, Cullin3, CSNS/Jab1, Nrf2 (C-20), BCL-2, BCL-XL, HSP70 and BAK were obtained from Santa Cruz. Membranes were probed with specific antibodies and protein quantity visualized using an Odyssey instrument (Li-CoR Bioscience). Images have been cropped for presentation.

Isolation of crypts, lamina propria lymphocytes (LPLs) and flow cytometry analysis. Small intestinal crypts were isolated as described. In brief, the small intestine was removed and lumen was rinsed with ice-cold PBS (Mg⁻/Ca⁻). Using a microscope cover slide, firmly scrape the gut lumen to remove villi and mucus, of 2-3 mm were shaken in Hank's balanced salt solution (HBSS) two times and then placed in HBSS containing 10 mM EDTA on ice for 30 min. Tissue pieces were shaken and contents collected were filtered twice through a 70 μm strainer (BD) to remove villous material. At this point, the suspension was mainly composed of crypts and the number of crypts was estimated by hemocytometry. The method used for isolation of LPLs has been previously described. In brief, fat tissues and Peyer's patches (PPs) were removed from small intestine. The intestine was open and cut in pieces 1-cm long and incubated in an HBSS solution containing 5 mM EDTA and 10 mM Hepes) for 30 min at 37° C. with slow rotation (180 r.p.m. min⁻¹). Pieces were then further cut and incubated in an HBSS solution containing 0.5 mg ml⁻¹ DNase I (Roche) and 1 mg ml⁻¹ Collagenase type IV (Worthington). A discontinuous Percoll separation method (40 and 75%) was used to purify immune cells. Cell suspensions were centrifuged and the pellet was resuspended in 40% of Percoll layered by 75% of Percoll (GE Healthcare). The cells concentrated at the interface were collected and washed in cold PBS solutions. For flow cytometry analysis, the cells were labelled using standard procedures described above.

Microarray Analysis. Total RNA from crypt was isolated using the RNeasy Mini Kit (Qiagen, Hilden, Germany) according to the manufacturer's protocol (n=3 RNA samples per genotype). For microarray whole transcript expression analysis, 250 ng of total RNA was amplified and labeled following the GeneChip® WT PLUS Reagent kit protocol from Asymetrix (ThermoFisher, Waltham, Mass.), followed by hybridization to Asymetrix Mouse Clariom™ S arrays. The arrays were processed following the manufacturer recommended wash and stain protocol on an Asymetrix FS-450 fluidics station and scanned on an Asymetrix GeneChip® 7G scanner using Command Console 4.0. The resulting .cel files were imported into Partek Genomics Suite 6.6 and transcripts were normalized on a gene level using RMA as normalization and background correction method. A 1-way ANOVA was set up to compare CSN8^{flox/flox} and CSN8^{ΔIEC}. Step-Up False Discovery Rate was chosen as multiple test correction for the resulting p-values. Genes whose fold-change was over 1.5 in both comparisons were inputted into The Database for Annotation, Visualization and Integrated Discovery (DAVID) v6.7 for pathway analysis. Genes found to be significantly enriched (p<0.05) for Paneth cells function were then plotted in a heatmap using R software.

Electron microscopy. Two-millimeter ileum pieces were fixed in 2.5% glutaraldehyde in 0.1 M Sorenson's phosphate buffer and kept at 4° C. After several thorough washes in 0.1 M Sorenson's phosphate buffer, the blocks were postfixed in 1% osmium tetroxide and washed in water before staining with uranyl acetate. After inclusion in epoxy resin, 300-400 ran semithin sections were first realized to control the good orientation of the samples; then 80-90 ran sections were cut on a Reichert Ultracut E ultramicrotome. The ultrathin sections were transferred onto 150-mesh copper grids before staining with uranyl acetate and lead citrate. The sections were then viewed under a JEOL 1011 transmission electron microscope with a GATAN Erlangshen CCD camera.

Bacterial culture. Colonic mucosa and fecal pellets were removed and suspended in the 1 ml PBS by vortexing and by bashing with a sterile bacteriological loop. The suspension was then diluted and plated on MacConkey II agar and Columbia CNA with 5% sheep blood agar. The aerobic agar plates were incubated in 37° C. for 24 hours. Total anaerobic numbers were determined by culturing diluted samples on Brain Heart Infusion (BHI) agar plates (BD Biosciences) supplemented with L-cystine (0.5%), vitamin K (0.5 mg/L) and hemin (0.5 mg/L). Agar plates were incubated anaerobically (10% H₂, 80% N₂, and 10% CO₂) for 96 hr to enumerate total anaerobic bacteria. After incubation the numbers of colonies on the plates were counted and the number of bacteria per mg of feces was calculated.

16S rRNA Gene Analysis of Bacteria. Total DNA was extracted from murine fecal pellets and mucosal samples using QIAamp DNA Stool Mini Kit (Qiagen). DNA concentration was determined spectrophotometrically by using a Nanodrop instrument (Thermo Scientific) and analyzed by quantitative real time PCR using the bacterial specific primers 28 listed (Table 2). QPCR was run using the BioRad CFX96 qPCR System with each reaction run in triplicate. Analysis and fold-change were normalized to the amount of total 16S rRNA in the sample using the comparative threshold cycle (Ct) method. For 16S rRNA gene sequencing, bacterial DNA from fecal samples was isolated with QIAamp DNA Stool Mini Kits (Qiagen). 15 ng of DNA was used as template to amplify 16S rRNA gene using High Fidelity PCR system kit (Roche). The v1-v3 regions of 16S ribosomal RNA gene was amplified using 27f (AGAGTTTGATCCTGGCTCAG; (SEQ ID NO: 84) and 534r (ATTACCGCGGCTGCTGG; SEQ ID NO: 66) primers (1 μM). The primers were anchored with adaptor (adopter A: 5′ CCATCTCATCCCTGCGTGTCTCCGACTCAG 3′ (SEQ ID NO: 85) and adopter B: 5′ CCTATCCCCTGTGTGCCTTGGCAGTCTCAG 3′ (SEQ ID NO: 86)) and Multiplex Identifiers (MIDs; 10 bp long). The following PCR protocol were used for amplifying fragments: 95C for 3 min, followed by 27 cycles of 95C for 15 s, 58C for 15 s, 72C for 15 s, and a final extension at 72C for 5 min. PCRs products were pooled and purified on a 1% TAE ultrapure agarose gel using purification columns (Qiagen) for the generation of Illumina libraries. The amplicon sequence was conducted using the 454 Jr. Sequencing platform. The 16S rRNA gene sequences were analyzed using QIIME platform scripts (www.qiime.org). The sequences were verified at randomly selected 1500 sequences/sample and downstream analysis was performed. The microbial classification was performed using GreenGenes reference data base (gg_otus-13_8) using QIIME tools. The sequences reference picked into Operational Taxonomic Units (OTUs) by clustering 97% sequence similarity (uclust) and classified at various taxonomic ranks (phylum, order, class, family, genus, and species). The beta diversity principle co-ordinate plots were generated using phylogenetic metrics of UniFrac distances. The phylogenetic analysis was performed using Figtree with default parameter. The evolutional tree and percentage for each bacterial species were virtualized by Interactive Tree Of Life (iTOL) software. The LEfSe (linear discriminant analysis effect size) algorithm was applied for discovery of high-dimensional biomarkers that discriminate between APC^{Min/+}CSN8^{fl/fl} and APC^{Min/+}CSN8^{ΔIEC} mice.

Polyamine pools and Enzyme analyses. Tumor tissue was removed from intestine and was snap-frozen and stored at −70° C. Following removal of tumors, the normal small intestinal and colonic mucosa was scraped with a glass slide and snap-frozen. Frozen tissues were crushed into a fine powder. Specimens were homogenized in 1.2N perchloric acid and centrifuged to obtain the soluble supernatant extracts for polyamine analyses. Intracellular polyamine pools and acetylated polyamine pools were extracted with 1.2N perchloric acid, dansylated with dansyl chloride. The derivatized polyamines were separated on and analyzed using high-performance liquid chromatography on 150×4.6 mm ZORBAX SB-C18 column (Agilent) using methods described elsewhere. Polyamines were also quantitated by ELISA kit (Mybiosource, # MBS094198). For analysis of the enzyme activity, intestinal mucosal samples (4 mm²) were immediately transferred to vials containing 0.5 ml of an ice-cold buffer [50 mM Tris-HCl (pH 7.5), 0.1 mM EDTA, and 0.1 mM pyridoxal phosphate)] and then homogenized and centrifuged. SSAT and ODC activities were determined by using C-labeled substrates and scintillation counting of end products produced as described previously (31) (or Cancer research SSAT mice).

### Example 8—Deletion of CSN8 in IEC Leads to Higher Susceptibility to DSS-Induced Intestinal Inflammation

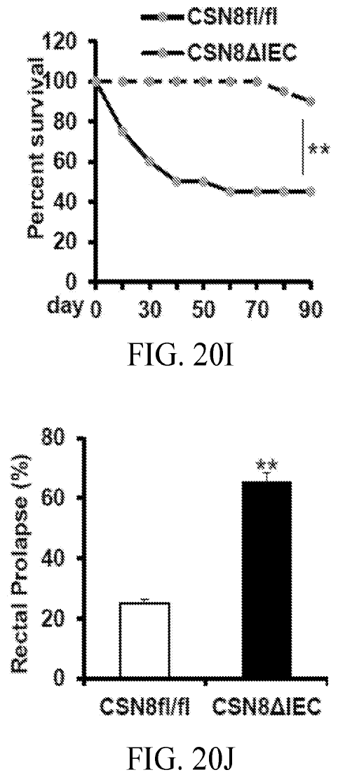

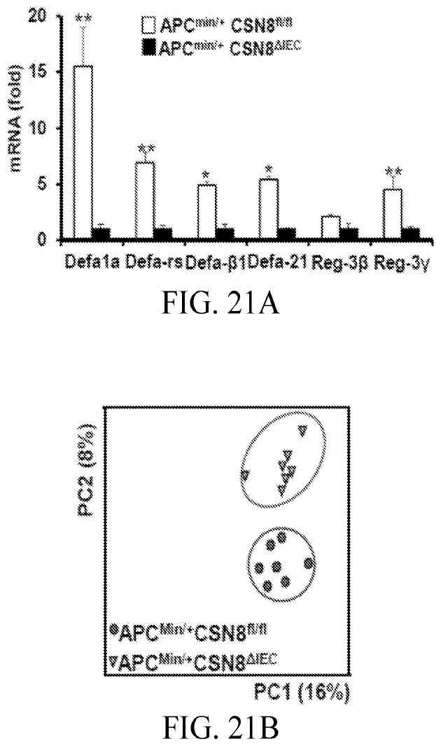

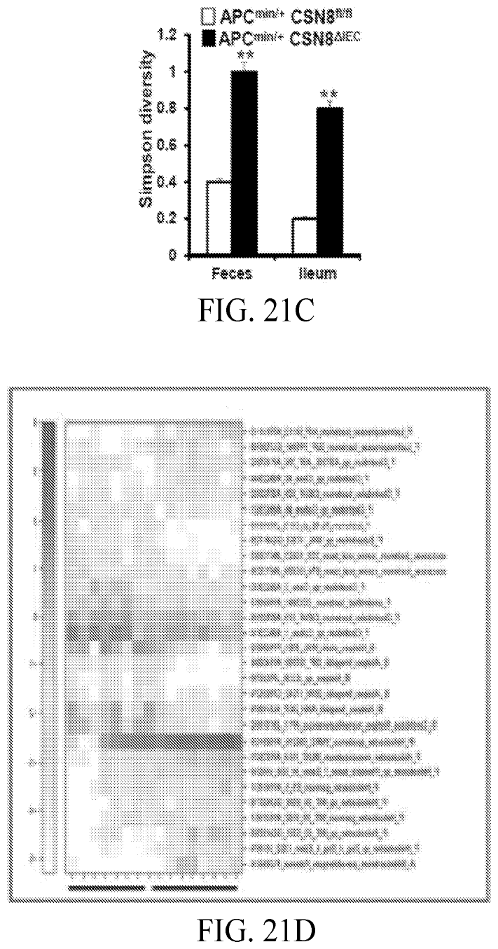

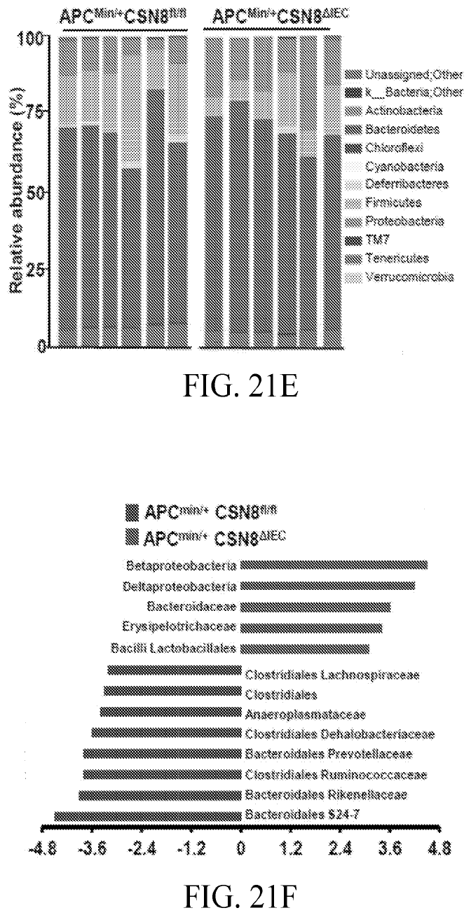

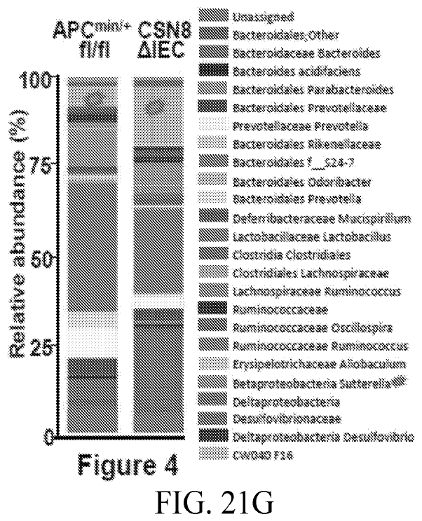

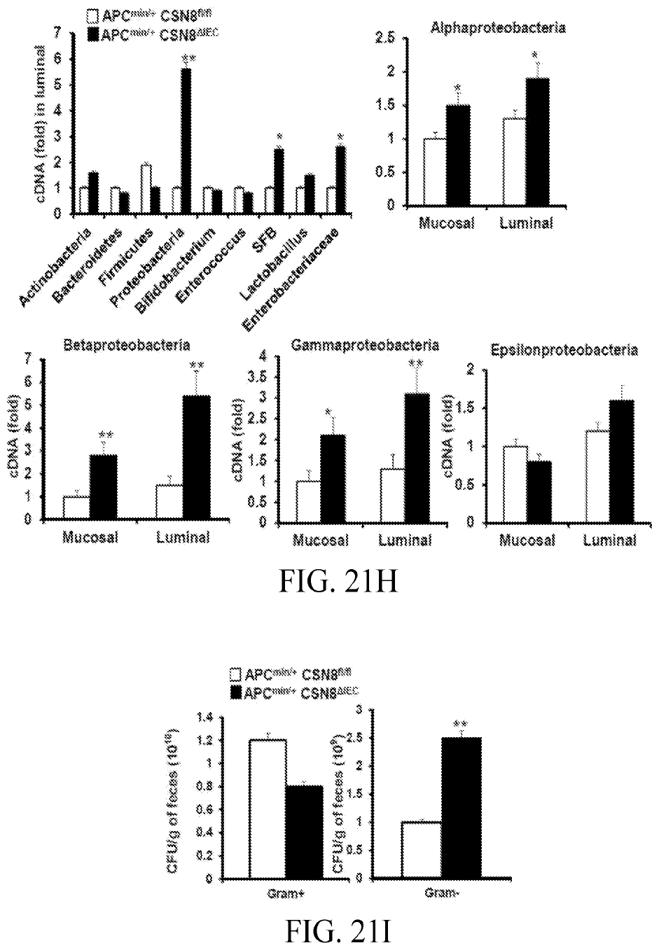

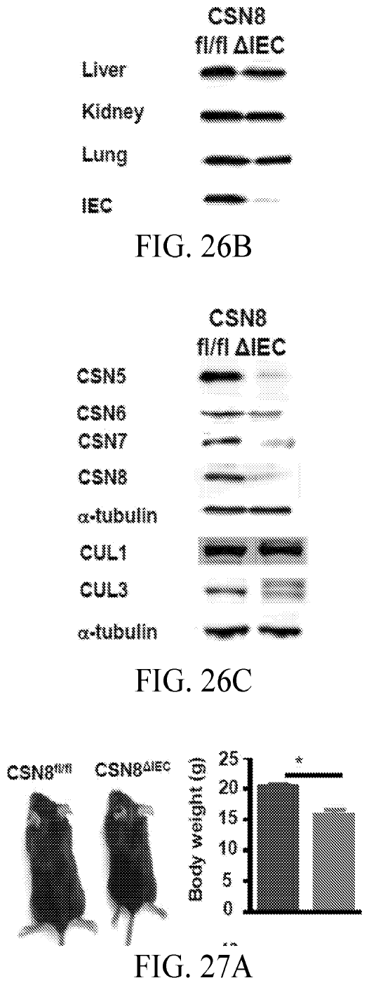

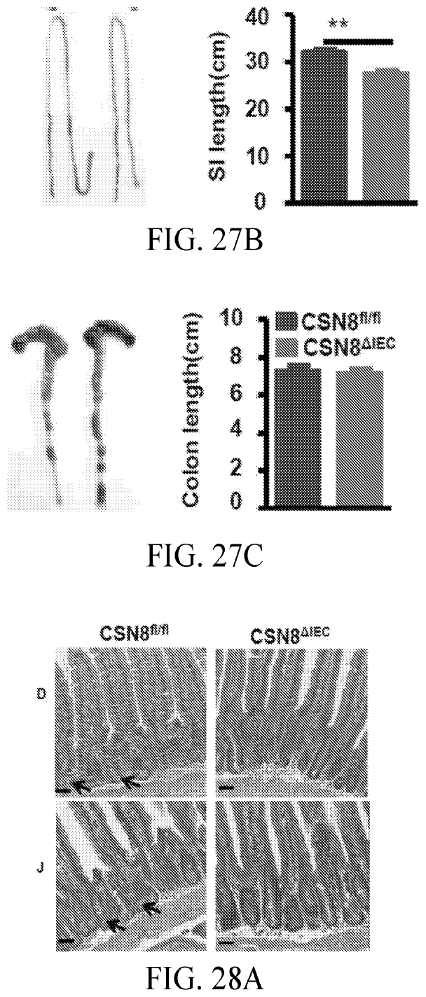

Cop9 signal some (CSN) regulates cell cycle and proliferation, and gut epithelial cell renewal takes place every 7 days in mice and 10 days in human. To address the in vivo role of CSN8, which is one of subunit of COP9 in the intestinal epithelium, mice expressing Villin-Cre and CSN8-lox alleles (GSM8^{fl/fl}) were generated to knockout CSN8 in intestinal epithelial cells (IEC) (CSN8^{ΔIEC}). To verify the cell-type specificity of CSN8 knockout, immunofluorescence staining (IF) was performed for CSN8 along the anterior-posterior axis of the mouse small and large intestine. In control CSN8^{fl/fl} intestine, CSN8 is expressed in both intestinal epithelial cells and interstitial cells (FIG. 26A). In CSN8^{ΔIEC} intestine, however, the majority of crypt/villus units are negative for CSN8 while all the interstitial cells remain positive for CSN8 (FIG. 26A). The efficiency of CSN8 deletion in isolated IEC was confirmed by western blotting (FIG. 26B). In addition, CSN8 protein levels were examined in other organs and no changes were detected in liver, kidney and lung of CSN8^{ΔIEC} mice, compared with the controls (FIG. 26B). These results showed that the deletion of CSN8 is specific to intestinal epithelial cells. It has been reported that CSN8 deletion or knockdown can cause instability of other CSN subunits. It was found that CSN5, CSN6 and CSN7 protein amounts were also decreased to various degrees (FIG. 21C), indicating the essential role of CSN8 for the COP9 complex integrity in IECs. The best known function of COP9 complex is the regulation of Cullin-Ring-E3 ubiquitin ligase activity through deneddylation. Consistent with the loss-of-function mutants of individual CSN subunit, CSN8-deficient IECs displayed a marked increase of neddylated cullins (FIG. 26C), indicating that the cullin deneddylation activity was compromised. In addition, CSN8^{ΔIEC} mice displayed a significant reduction in body weight (FIG. 27A) and small intestinal length while colon length remained the same (FIG. 27B-27C).

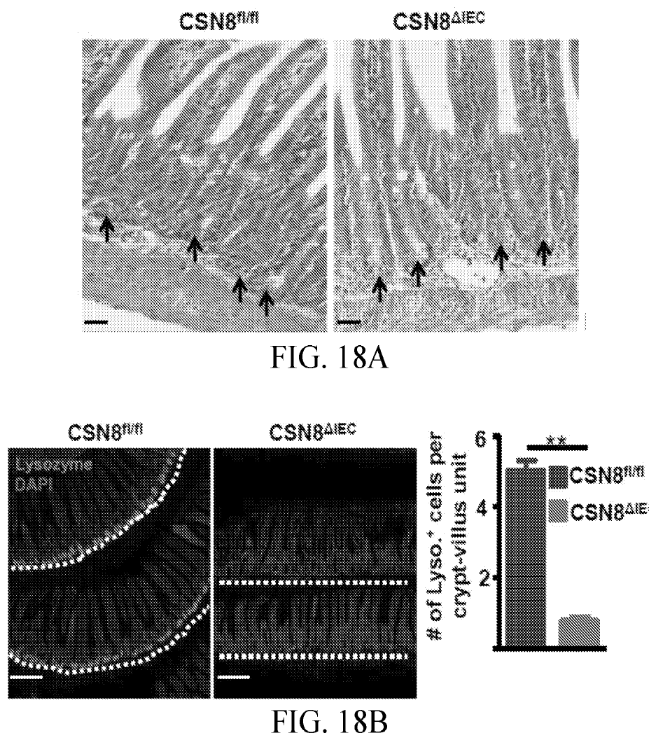

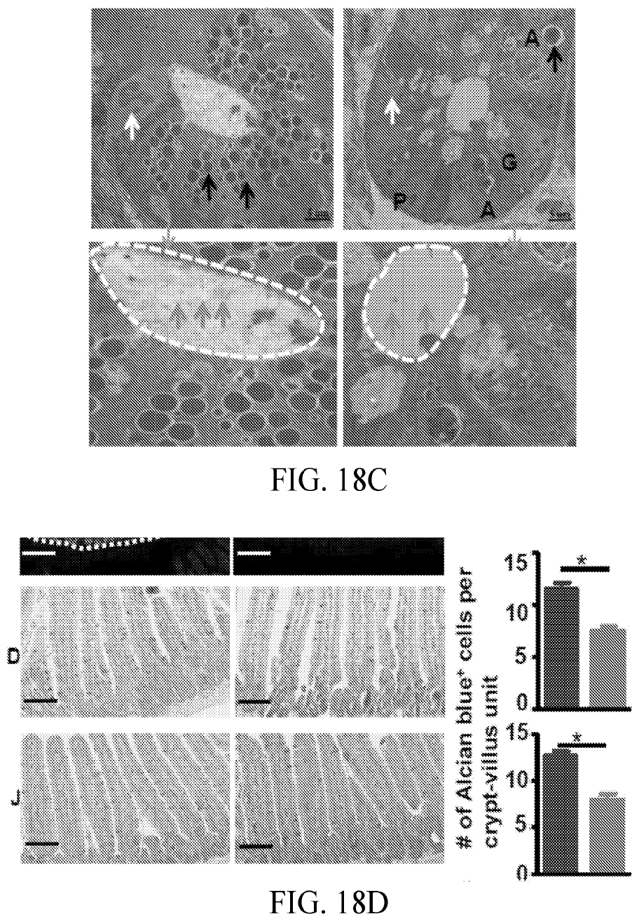

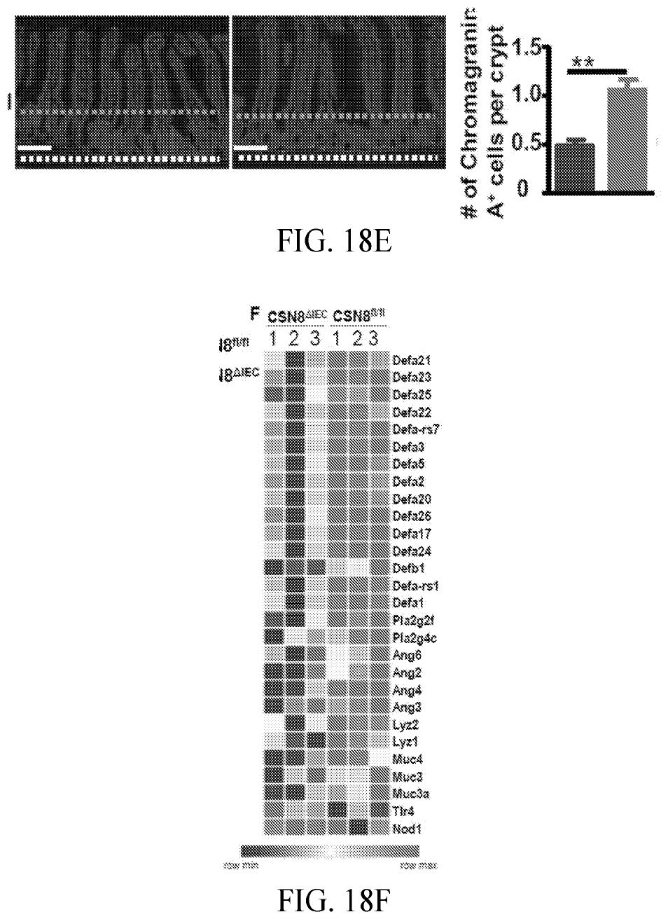

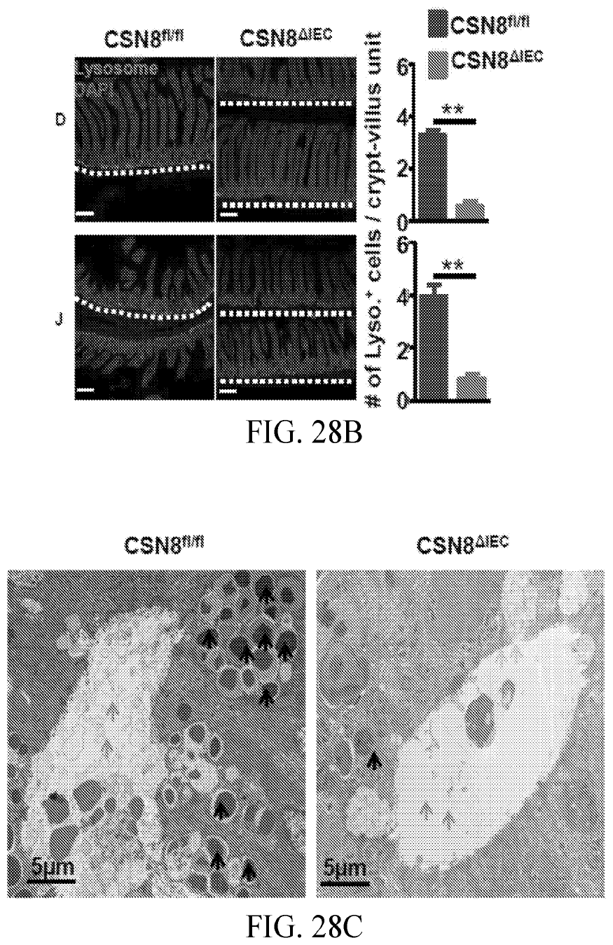

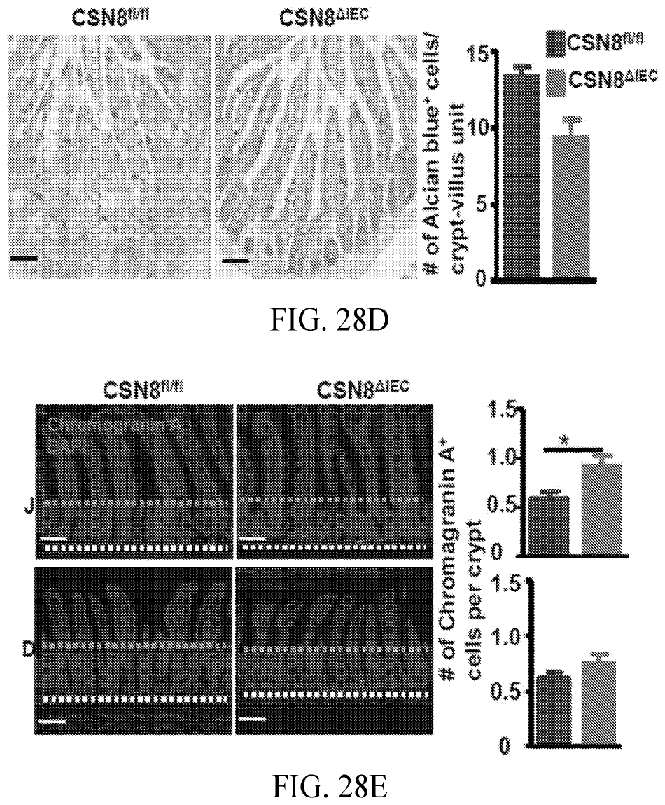

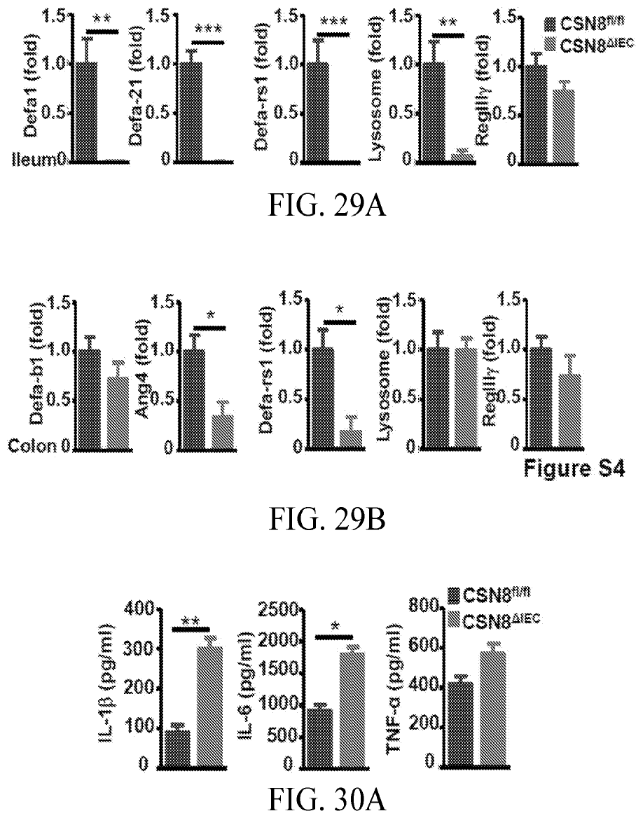

Histological analysis revealed a sharp reduction of Paneth cells throughout the length of the small intestine (FIG. 18A and FIG. 28A). Paneth cell granules store lysozyme, which were barely detectable in CSN8^{ΔIEC} crypts (FIG. 18B and FIG. 28B). Electronic microscopy (EM) showed that the presence of degenerating organelle membranes and loss of granules in Paneth cells of CSN8^{ΔIEC} mice (Black arrows in FIG. 18C). Although numerous secretory granules are apically located, secreted into the cryptic lumen of CSN8^{fl/fl} mice, a complete absence of secretory granules in the lumen of CSN8^{ΔIEC} mice was observed (Blue arrows in FIG. 18C and FIG. 28C). EM analysis also revealed microvilli were greatly lined in the inner surfaces of cryptic lumina in CSN8^{fl/fl} mice, but markedly reduced in CSN8^{ΔIEC} mice (Yellow arrows in FIG. 28C). These data indicate that CSN8 contributes to Paneth cell homeostasis. EM exhibited smaller cytoplasmic mucin droplets and a contracted ER in CSN8^{ΔIEC} goblet cells (white arrow in FIG. 18C). The number of alcian blue⁺ goblet cells mildly reduced in duodenum and jejunum (FIG. 18D) but not in ileum (FIG. 28D). Though the number of enteroendocrine cells remained unaffected, chromogranin A⁺ (ChgA⁺) staining revealed a clear mislocalization of ChgA⁺ cells (FIG. 18E and FIG. 28E). Instead of migrating upwards to the villi as in control mice, the majority of ChgA⁺ enteroendocrine cells migrated downwards to the bottom of crypts where stem cells and Paneth cells reside in CSN8^{ΔIEC} mice (FIG. 18E and FIG. 28E). Paneth cells are one of the types of cells releasing antimicrobial peptides (AMPs) which play a protective role against the pathogenesis of IBD. To test whether CSN8 deficiency triggers dysbiosis via defects in the production of AMPs, the global gene expression profiles were examined in crypt IECs by Clariom S sDNA array. mRNAs panel of AMPs produced in CSN8^{ΔIEC} and CSN8^{fl/fl} mice were analyzed. Consistent with the marked reduction of Paneth cells, the expression of defensins was barely detectable and the expression of lysozyme was also dramatically reduced in IECs of distal ileum of CSN8^{ΔIEC} (FIG. 18F). Real-time PCR confirmed that the expressions of antimicrobial peptides Defa-1, Defa-21, Defa-b1, Defa-rs1 and Ang4 were reduced significantly in ileum and colon (FIG. 29A and FIG. 29B).

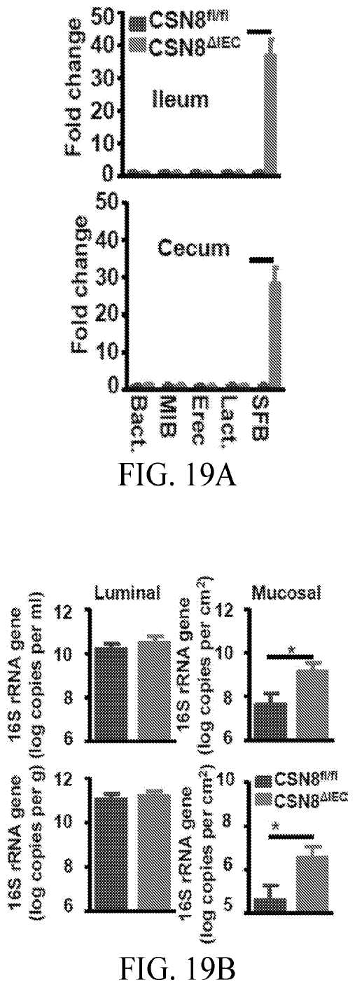

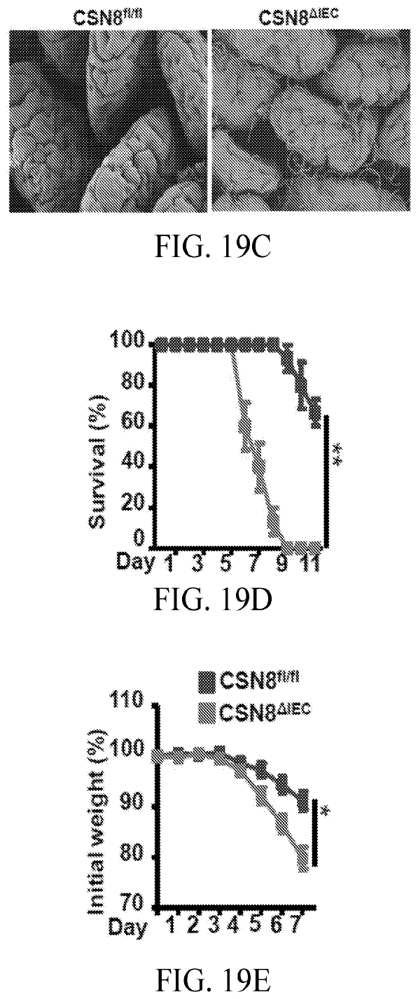

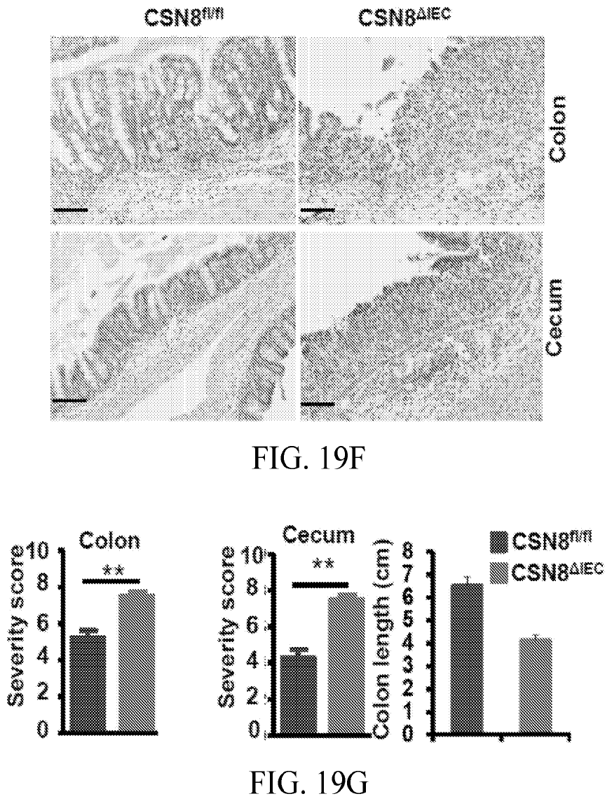

Susceptibility to intestinal inflammation is often associated with alterations in commensal bacterial populations. Using qPCR with 16S rRNA gene primers targeting groups covering the dominant bacterial populations in the mouse intestinal tract, the relative abundance of individual bacterial groups in distal ileum and cecum of CSN8^{ΔIEC} mice and their single housed controls was compared. These included members of the gram-positive firmicutes phylum (Eubacterium rectale, Lactobacillus, and Segmented Filamentous Bacteria (SFB) groups) and the gram-negative bacteroidetes phylum (Bacteroides and Mouse Intestinal Bacteroides (MIB) groups). Surprisingly, though the relative number of the gram-negative Bacteroides and MIB and gram-positive Eubacterium rectale and Lactobacillus remained similar, there was a dramatic increase of SFB (FIG. 19A). The altered composition of bacteria species as a result of knockout of CSN8 in gut epithelial cells may be due to their different spatial distribution, luminal vs. anchoring the apical surface of epithelia. Bacteria in the mucus versus lumen was quantitatively analyzed using qPCR. Although the luminal bacterial loads didn't show significant difference, the bacteria recovered from the intestinal surface showed a consistent higher trend in CSN8^{ΔIEC} mice as compared with CSN8^{fl/fl} mice (FIG. 19B). Indeed, Scanning electron microscopy also revealed a thicker network of bacteria in the distal ileum of CSN8^{fl/fl} mice comparing to that of CSN8^{fl/fl} mice (FIG. 19C). These results indicated that knockout of CSN8 in gut epithelial cells leads to more bacteria accessing the gut mucus, and higher dense mucus associated bacteria due to knockout CSN8 is agreeable with the fact that reduction of expression of the genes encoding for antimicrobial peptides secreted by Paneth cells.

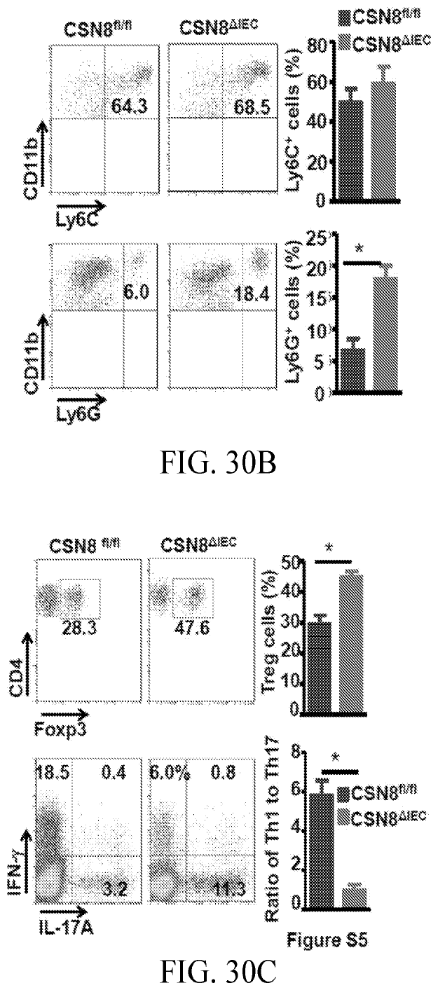

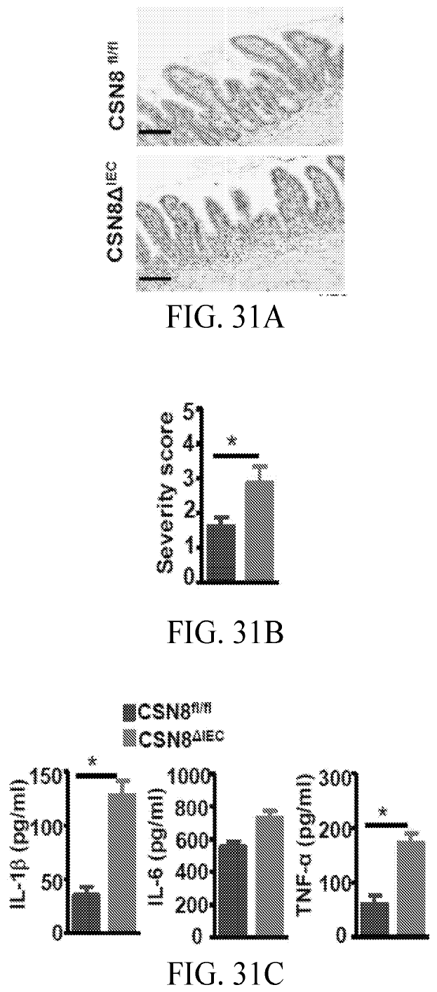

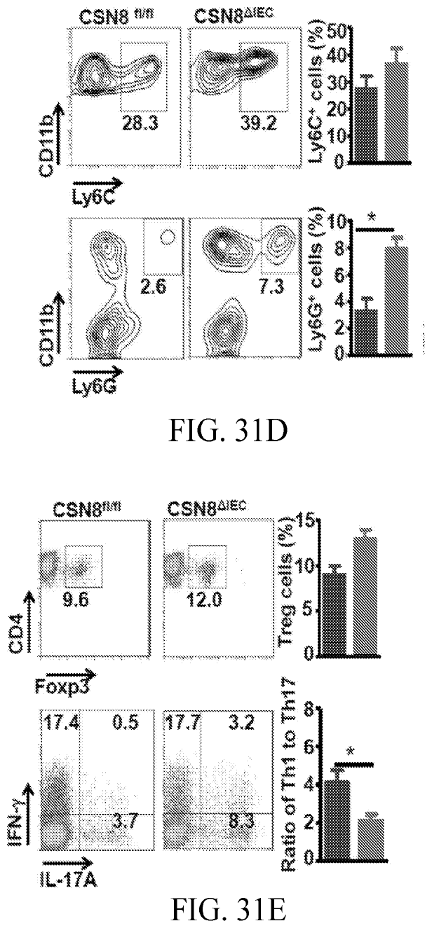

Increasing the mucus associated bacteria play a role in gut inflammation. To assess the significance of IEC-intrinsic CSN8 expression in the context of intestinal damage and inflammation, DSS induced mouse colitis model was used. After administration of lower concentration of DSS (2%) for 7 days, CSN8^{fl/fl} mice showed significantly increased mortality and severe weight loss (FIG. 19D and FIG. 19E) compared to CSN8^{fl/fl} mice. Histological examination of CSN8^{ΔIEC} large intestine revealed severe inflammation with massive cellular infiltration into the mucosa and loss of crypts and surface epithelium, particularly in cecum (FIG. 19F), which is in a sharp contrast to the minimally affected large intestine of CSN8^{fl/fl} mice. The severity of the colitis in CSN8^{fl/fl} mice was also reflected by the markedly increased colitis score and the shorten length of colon (FIG. 19G). Consistent with the increased inflammation, ELISA analysis of the CSN8^{ΔIEC} colon showed significantly increased secretion of proinflammatory cytokine IL-1β and IL-6 compared with control mice (IL-1β: 90.3±18.0 μg/ml vs. 299.9±26.4 μg/ml, P<0.01; IL-6:910.39±98.91 μg/ml vs. 1793.24±118.84 μg/ml, P<0.05) (FIG. 30A). The extent and types of cell infiltrating the inflamed large intestine were analyzed by flow cytometry. There were significantly more CD11b⁺Ly6G⁺ neutrophils in the colon of CSN8^{ΔIEC} mice (FIG. 30B). Correlated to severe inflammation, higher Treg frequency were observed LI-LP of CSN8^{ΔIEC} mice than that of control mice (28.3±2.8% vs. 47.6±1.8%, P<0.05) (FIG. 30C). The ratio of Th1 against Th17 cells, as indicated by the respective production of IFN-γ and IL-17 in CD4⁺ T cells, was much lower in CSN8^{ΔIEC} mice (FIG. 30C). Moreover, histological analysis of the distal ileum demonstrated notably loss of crypts at large area and increased cellularity in the lamina propria (FIGS. 31A-31B). The increased susceptibility to ileitis in the absence of CSN8 in IECs was further supported by increased production of proinflammatory cytokines IL-1β (35.30±7.93 μg/ml vs. 127.91±13.62 μg/ml, P<0.05) and TNF-α (60.19±15.39 μg/ml vs. 172.65±17.95 μg/ml, P<0.05) (FIG. 31C) and by elevated infiltration of the lamina propria with neutrophils (FIG. 31D) and Th17 cells (Th1/Th17 ratio: 4.11±0.24 vs. 2.11±0.18, P<0.05) (FIG. 31E). These data indicated that CSN8^{ΔIEC} mice were more susceptible than CSN8^{fl/fl} mice to intestinal inflammation, which is accompanied with deficiency in Paneth cells.

### Example 9—CSN8 Deletion Results in Inhibition of Tumor Development in Spite of Gut Inflammation

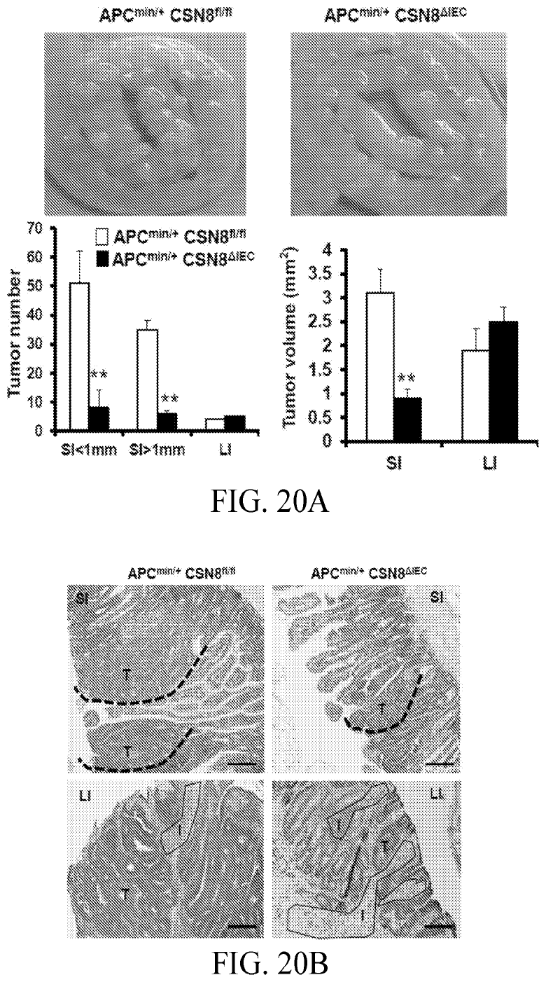

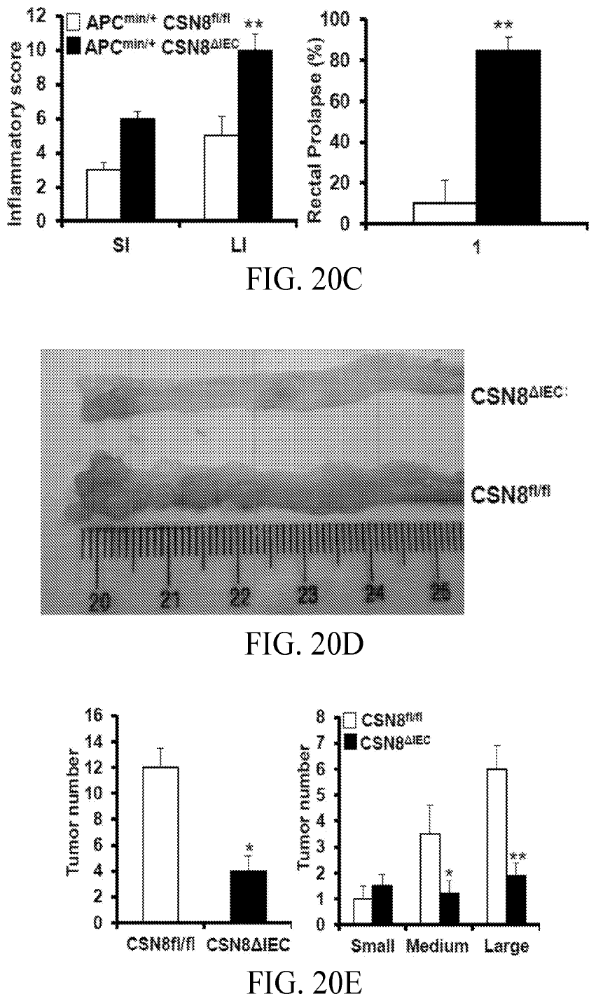

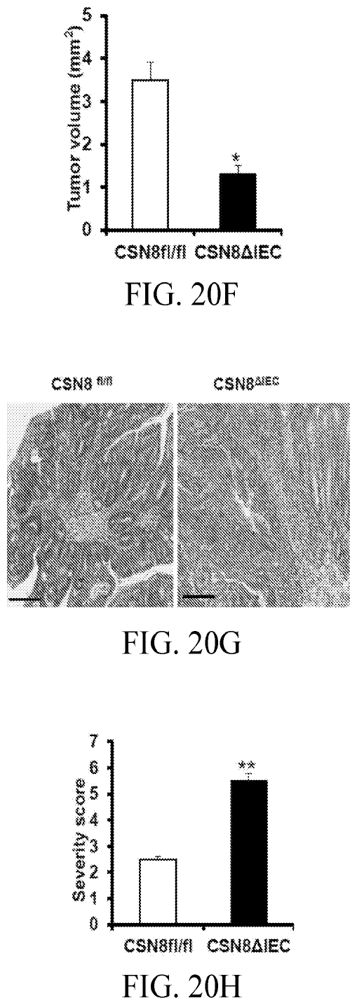

It is appreciated that altered expression of COP9 signalosome leads to promotion of tumor growth under sterile inflammation. Inflammation in general promotes tumor progression. In the above study, it was shown that CSN8 knockout leads to induction of gut inflammation. Next, it was tested whether CSN8 knockout has an effect on the development of mouse colon tumor with two colon tumor models. For the first model, CSN8^{ΔIEC} mice were bred into the tumor-prone genetic background of APC^{Min/+} mice. APC^{Min/+}CSN8^{fl/fl} mice showed clear signs of illness including loss of pale feet, body weight and hunching, and developed a large increase in the number and size of polyps in the small intestine when compared to APC^{Min/+}CSN8^{ΔIEC} sex-matched littermates (FIG. 20A). Total tumor counts in the small intestine were reduced by about 80% in APC^{Min/+}CSN8^{ΔIEC} mice; such a decrease was observed for both small (<1 mm diameter) and large (>1 mm diameter) tumors. Interestingly, there were no significant effects on the number or size of colonic polyps (FIG. 20A). Histopathological analysis demonstrated that the percentage of high-grade tumors was decreased in the tumors of APC^{Min/+}CSN8^{ΔIEC} mice (FIG. 20B). However, like the phenotype that was observed in CSN8 knockout (CSN8^{ΔIEC}) mice, APC^{Min/+}CSN8^{ΔIEC} mice displayed more severe mucosal inflammation, multifocal flat low-grade intraepithelial neoplasias in colon and frequent rectal prolapse than observed in APC^{Min/+}CSN8^{fl/fl} mice (FIGS. 20B-20C). It was then further tested whether the results generated from APC^{Min/+}CSN8^{ΔIEC} mouse model could be replicated in a carcinogen induced colon cancer model. To investigate the role of CSN8 in CAC, WT and CSN8^{ΔIEC} mice were exposed to the carcinogen azoxymethane (AOM) with dextran sodium sulfate (DSS). After the AOM/DSS challenge, CSN8^{ΔIEC} mice had both decreased tumor numbers and reduced tumor size in the distal to middle colon compared with WT mice (FIGS. 20D-20F). The average tumor number per mouse in WT mice was about three times higher than that in CSN8^{ΔIEC} mice (FIG. 20D). The large tumors (>2 mm diameter) in WT mice was about two folds lager than that in CSN8^{ΔIEC} mice (FIG. 20E). In contrast, the small tumors (<1 mm diameter) has no significant difference between the two groups (FIG. 20E). However, despite the decreased tumor growth, CSN8^{ΔIEC} mice display a significant extent of inflammation throughout the mucosa, loss of crypts, significantly elevated histopathological score, significant lower survival and severe relapse compared with WT mice (FIG. 20G-20K).

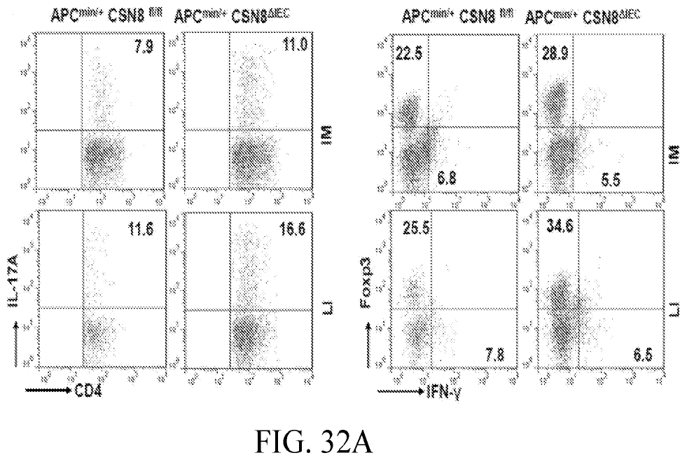

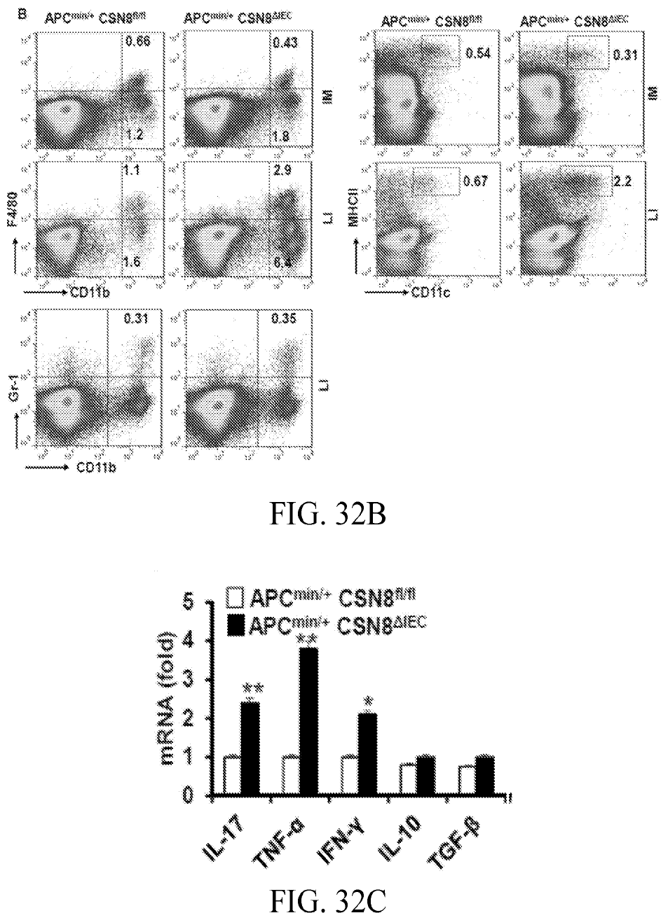

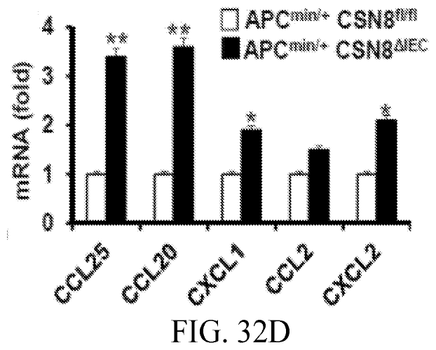

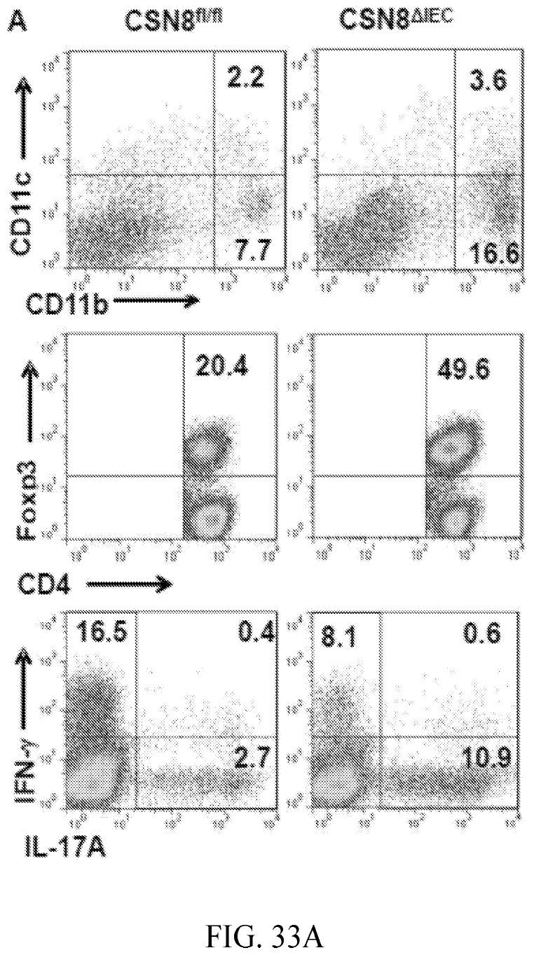

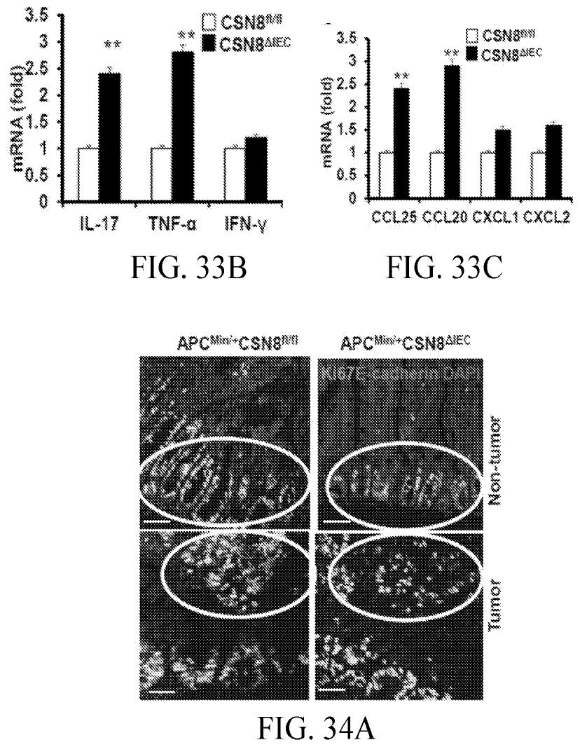

Both gut associated immune cells and cytokines play a crucial role in colon tumor development. To understand the role of CSN8 mediated inflammation, the immunological tumor milieu of APC^{Min/+}CSN8^{ΔIEC} was analyzed and the immunological analysis of AOM/DSS colitis was revisited in depth. Flow cytometric analysis revealed that the percentage of T regulatory and Th17 cells in the ileum and colon was higher in APC^{Min/+}CSN8^{ΔIEC} than in APC^{Min/+}CSN8^{fl/fl} mice, but IFN-γ-positive Th1 cells was similar (FIG. 32A). There were high percentages of myeloid cells including macrophages and dendritic cells in the colonic lamina propria of APC^{Min/+}CSN8^{ΔIEC} mice compared to APC^{Min/+}CSN8^{fl/fl} mice (FIG. 32B). The analysis of the chemokine milieu of the inflamed colon revealed a strong upregulation of various cytokines and chemokines such as IL-17A, CCL1, CCL25, CXCL1, CXCL2, and CCL20 in APC^{Min/+}CSN8^{ΔIEC} mice (FIGS. 32C-32D). To better assess the underlying inflammatory responses, an analysis of AOM/DSS colitis was further performed. In late phases of azoxymethane (AOM)/DSS model of CAC, there was an influx of CD11b⁺ cells and lymphocytes in the intestinal mucosa of CSN8^{ΔIEC} mice associated with higher levels of inflammatory cytokines within the lamina propria of CSN8^{ΔIEC} than in CSN8^{fl/fl} mice (FIGS. 33A-33B). In addition, it was observed that the transcript levels of the genes associated with chemokines CCL20 and CXCL1 in the intestine were higher in CSN8^{ΔIEC} than in CSN8^{fl/fl} mice (FIG. 33C). In summary, these results indicated that intestinal epithelial CSN8 deficiency diminishes tumor development and growth but induces intestinal inflammation.

### Example 10—Induced Intestinal Inflammation is Accompanied with Altered Fecal Gut Microbiota Composition in APC^{Min/+}CSN8^{ΔIEC} Mice

Paneth cells are a source for releasing antimicrobial peptides and preventing dysbiome. CSN8 knockout leads to reduction of generation of Paneth cells. Next, it was analyzed whether Paneth cell dysfunction contributes to the changes in the expression of antimicrobial peptides and in the composition of the intestinal microbiome in the context of APC^{Min/+}CSN8^{ΔIEC} gut microenvironment. The results of real-time PCR confirmed that expression of multiple antimicrobial peptides was also markedly reduced in isolated gut ileum epithelial cells from APC^{Min/+}CSN8^{ΔIEC} mice (FIG. 21A). A 16S rRNA gene-based microbiota sequencing analysis showed that the fecal microbiota derived from APC^{Min/+}CSN8^{ΔIEC} mice clustered apart from those of APC^{Min/+}CSN8^{fl/fl} control (FIG. 21B). The microbial composition was striking different between APC^{Min/+}CSN8^{fl/fl} and APC^{Min/+}CSN8^{ΔIEC} mice and significantly altered in APC^{Min/+}CSN8^{ΔIEC} mice (FIGS. 21C-21D). Out of 11,158 operational taxonomic units, 58 bacterial species were differentially represented in faeces between APC^{Min/+}CSN8^{fl/fl} and APC^{Min/+}CSN8^{ΔIEC} mice (FIG. 21D). Intriguingly, metagenomic analysis of gut microbiota revealed that the abundance of Bacteriodetes is similar in APC^{Min/+}CSN8^{fl/fl} mice and APC^{Min/+}CSN8^{ΔIEC} mice. However, CSN8 deficiency led to a high abundance of Gram⁻ bacteria related to Proteobacteria and lower abundance of Gram⁻ bacteria related to Firmicutes in faeces (FIG. 21E). Notably, linear discriminant analysis effect size (LEfSe) analysis identified beta-proteobacteria mostly from Gram⁻ proteobacteria were specific of the microbiota of APC^{Min/+}CSN8^{ΔIEC} mice (FIG. 21F), whereas the particular Clostridiales related to Gram⁺ Clostridia were relatively higher in APC^{Min/+}CSN8^{fl/fl} mice (FIG. 21F). In addition, the faecal samples of APC^{Min/+}CSN8^{ΔIEC} mice were mainly colonized with microbial communities related to the beta-proteobacteria Sutterella (FIG. 21G). 16S rDNA real-time PCR further confirmed that the bacterial numbers of the enterobacteriaceae and SFB were slightly increased in the luminal specimens. Notably, the number of Proteobacteria phylum bacteria, particular Betaproteobacteria, was dramatically increased both in the luminal specimens and at the mucosal surfaces in APC^{Min/+}CSN8^{ΔIEC} mice (FIG. 21H). Gut bacteria culture analysis of colonic mucosa samples confirmed that the Gram⁻ bacteria was lower in APC^{Min/+}CSN8^{fl/fl} mice than in APC^{Min/+}CSN8^{ΔIEC} mice (FIG. 21I).

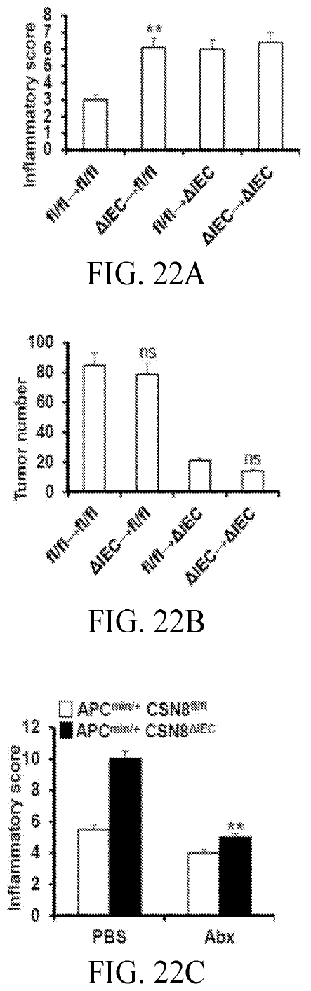

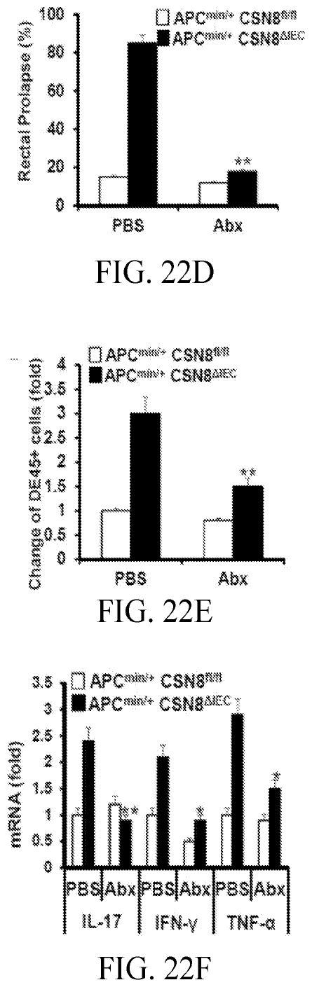

To determine whether bacterial dysbiosis play a causal role in the exacerbated intestinal inflammation and/or reduced tumorigenesis in APC^{Min/+}CSN8^{ΔIEC} mice, fecal transfer experiments were performed with APC^{Min/+}CSN8^{ΔIEC} and APC^{Min/+}CSN8^{fl/fl} mice to study the alterations in inflammation and tumor growth. APC^{Min/+}CSN8^{fl/fl} CSN8^{fl/fl} mice that were received with microbiota from APC^{Min/+}CSN8^{fl/fl} CSN8^{ΔIEC} mice showed more intestinal inflammation, but similar tumor growth compared with the mice only received with APC^{Min/+}CSN8^{fl/fl} microbiota, as unveiled by a higher histological score (FIGS. 22A-22B). Despite these differences in induction of an inflammatory response, transfer of fecal from APC^{Min/+}CSN8^{fl/fl} mice had no effect on intestinal inflammation and tumor growth in APC^{Min/+}CSN8^{ΔIEC} mice (FIGS. 22A-22B). To test whether microbiota directly affected intestinal inflammation in APC^{Min/+}CSN8^{ΔIEC} mice, mice were treated by antibiotics. Antibiotic treatment led to a large decrease in both inflammation scores and rectal prolapse in the colon of APC^{Min/+}CSN8^{ΔIEC} mice (FIGS. 22C-22D). Strikingly, antibiotic suppressed both immune cells infiltration (FIG. 22E) and pro-inflammatory cytokines production (FIG. 22F) in the ileum of APC^{Min/+}CSN8^{ΔIEC} mice. This data indicated that the more severe intestinal inflammation in APC^{Min/+}CSN8^{ΔIEC} mice is likely due to dysbiosis gut microbiota induced intestinal inflammation. However, gut microbiota from APC^{Min/+}CSN8^{ΔIEC} mice do not seem contribution to promoting colon tumor development.

### Example 11—Decreased Tumor Growth is Associated with Altered Polyamine Metabolism in APC^{Min/+}CSN8^{ΔIEC} Mice

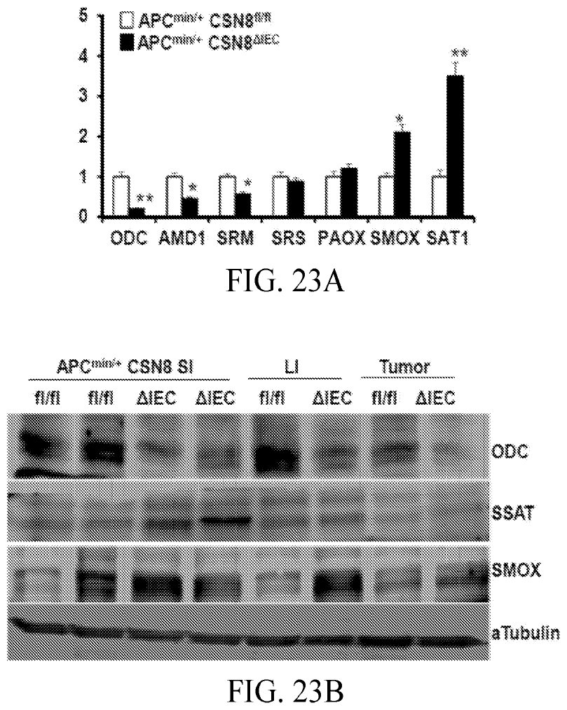

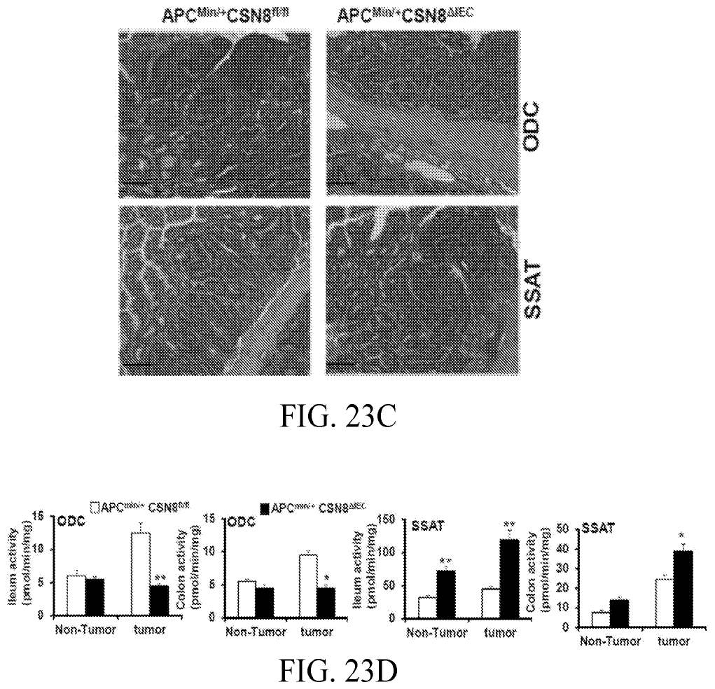

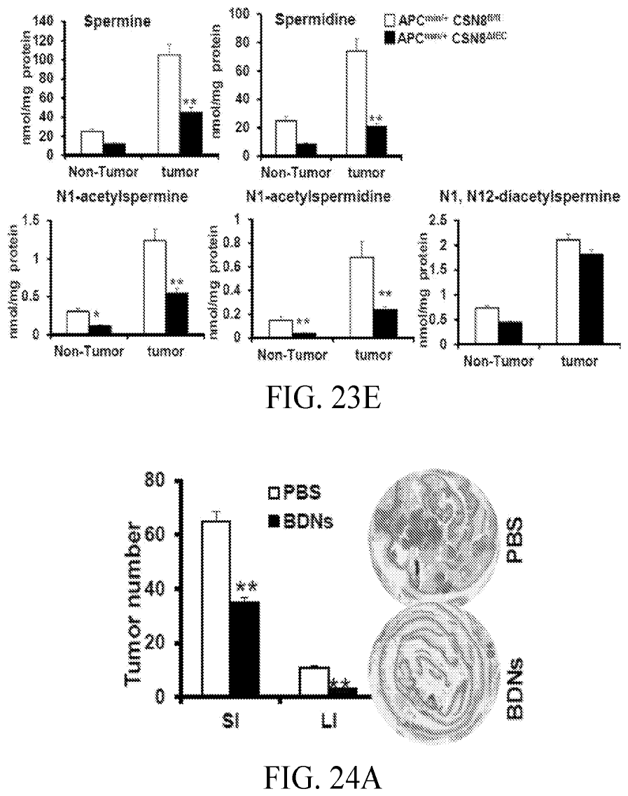

COP9 regulates cell cycling, growth, and immune response via regulation of a number of pathways at transitionally and posttranscriptional levels. The present data indicated that knockout of CSN8 causes instability of COP9. To systematically understand the role of gut CSN8 in intestinal inflammation and tumor growth, the global gene expression profiles were further examined in crypt IECs. The most interesting finding is that IECs from APC^{Min/+}CSN8^{ΔIEC} mice have a decreased numbers of genes associated with the polyamines pathway which is under control of ODC and feedback from polyamines. Remarkably, changing polyamines enzymes involved biosynthetic and catabolic pathways, including ODC, AMD1, SRM and SAT1 were observed in the data generated from both protein array and cDNA array data. Real-time PCR analysis of IECs in ileum showed that the expression of ODC, AMD1, SRM and spermine synthase (SMS) were generally repressed in APC^{Min/+}CSN8^{ΔIEC} mice, whereas mRNAs encoding enzymes in the catabolic arm of the pathway, spermine/spermidine N-acetyltransferase (SAT1) and spermine oxidase (SMOX) were elevated in APC^{Min/+}CSN8^{ΔIEC} mice (FIG. 23A). Ileal nuclear extracts from APC^{Min/+}CSN8^{fl/fl} mice and APC^{Min/+}CSN8^{ΔIEC} mice were also analyzed by Western blotting to detect differences in ODC, SSAT and SMOX expression. In the ileum, decreased ODC protein and increased nuclear SSAT protein levels were found in APC^{Min/+}CSN8^{ΔIEC} compared with APC^{Min/+}CSN8^{fl/fl} (FIG. 23B). In addition, immunohistochemical (IHC) staining showed that a significant difference was detected in mucosal SSAT and ODC expression between APC^{Min/+}CSN8^{fl/fl} mice and APC^{Min/+}CSN8^{ΔIEC} (FIG. 23C). The activities of the two key polyamine enzymes ODC and SSAT were further measured in non-tumor mucosa and in tumors of the small intestine. ODC levels in APC^{Min/+}CSN8^{ΔIEC} mice were similar in non-tumor mucosa of both the small intestine and the colon relative to the APC^{Min/+}CSN8^{fl/fl} mice (FIG. 23D). Significant decreases were observed in tumors of the small intestine and the colon (FIG. 23D). However, SSAT activities in APC^{Min/+}CSN8^{ΔIEC} mice were significantly increased relative to APC^{Min/+}CSN8^{fl/fl} mice in the normal mucosa of the small intestine. SSAT activities were also found to be dramatically different in tumors between APC^{Min/+}CSN8^{fl/fl} mice and APC^{Min/+}CSN8^{ΔIEC} (FIG. 23D). To examine the effects of decreased CSN8 expression on polyamine pools, the primary polyamines, putrescine, spermidine, and spermine as well as the concentrations of the SSAT product N1-acetylspermidine, were measured and compared in APC^{Min/+}CSN8^{fl/fl} and APC^{Min/+}CSN8^{ΔIEC} mice (FIG. 23E). In normal small intestinal tissues, APC^{Min/+}CSN8^{ΔIEC} mice exhibited a>3-fold decrease in spermidine levels, and a 2-fold decrease in spermine and putrescine relative to APC^{Min/+}CSN8^{fl/fl} tissues (FIG. 23E). Even larger increases in both spermidine and spermine levels were observed in tumors of APC^{Min/+}CSN8^{fl/fl} mice relative to APC^{Min/+}CSN8^{ΔIEC} mice (FIG. 23E). However, no significant differences were found in levels of N1, N12-diacetylspermine spermidine in the normal intestine tissue or in tumor tissues between APC^{Min/+}CSN8^{fl/fl} mice and APC^{Min/+}CSN8^{ΔIEC}. By comparison, N1-acetylspermidine levels were significantly decreased in normal tissues and tumor in APC^{Min/+}CSN8^{ΔIEC} mice compared to APC^{Min/+}CSN8^{fl/fl} mice (FIG. 23E).

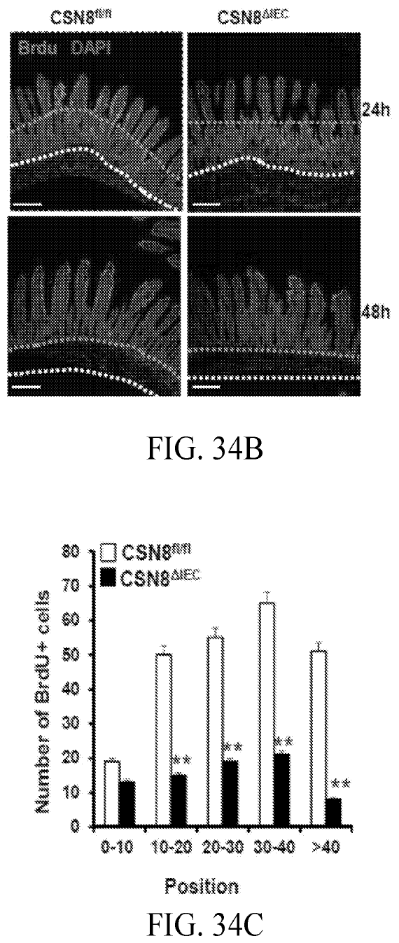

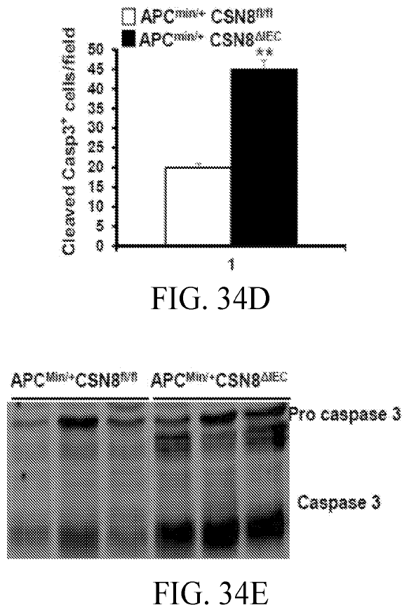

Both of the COP9 family and polyamines are considered important for normal cell cycle progression and dysregulation of COP9 and polyamines-mediated pathways promote cancer development. First, to determine whether APC^{Min/+}CSN8^{ΔIEC} mice have defect in cell proliferation, the levels of cell proliferation and apoptosis were analyzed. Strikingly, Ki-67 expression was reduced in the tumor and non-tumor tissue in the ileum of APC^{Min/+}CSN8^{ΔIEC} mice relative to controls (FIG. 34A). The effect of CSN8 deletion on the rate of proliferation of intestinal epithelial cells in vivo was further examined. At 3 weeks of age, mice were pulse-labeled with bromodeoxyuridine (BrdU) and sacrificed at 24 h and 48 h later. 24-hour pulse BrdU labeling revealed a significant decreased BrdU⁺ cell number in the small intestine of CSN8^{ΔIEC} mice compared to the control littermates (FIGS. 34B-34C). The rate of migration of epithelial cells as defined by the cumulative frequency of BrdU-positive cells along the crypt-villus axis was also lower in the small intestine of CSN8^{ΔIEC} mice compared to the control littermates at 24 h (FIGS. 34B-34C), indicating that CSN8 deficiency alone is associated with abnormal cellular proliferation. Strikingly, apoptosis analyses revealed a dramatically increased apoptotic cell number in CSN8-deficient tissue, indicating that cell division defects might have triggered increased cell apoptosis (FIGS. 34D-34E). Taken together, these data showed that APC^{Min/+}CSN8^{ΔIEC} IECs hypoproliferate relative to controls.

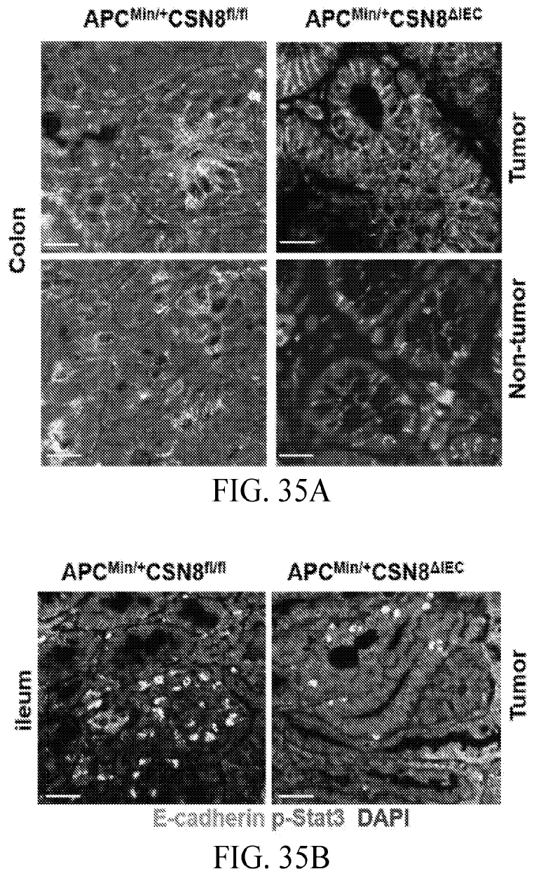

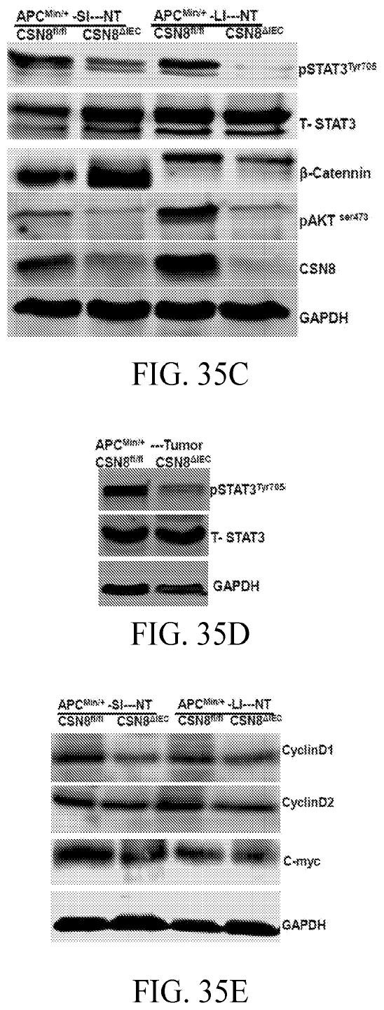

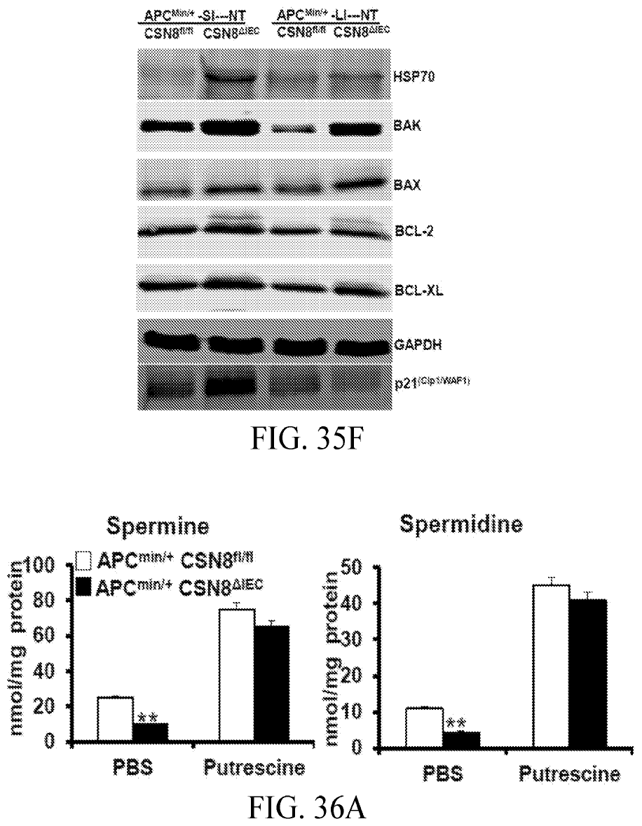

Because cellular polyamines are required in proliferating IECs during healing of damaged mucosa and STAT3 regulates the pathways associated with wound healing in IECs and acts as a key player linking inflammation and cancer during tumor development, the status of the polyamine-StaG signaling pathway in CSN8-deficient IECs was then further assessed. The high accumulation of nuclear Stat3 was observed in the non-tumor and tumor tissue of colon and ileum from APC^{Min/+}CSN8^{fl/fl} mice compared to that of APC^{Min/+}CSN8^{ΔIEC} mice (FIGS. 35A-35B). The results of western blotting also showed that activated STAT3 protein was reduced in intestinal epithelial cells and tumors in APC^{Min/+}CSN8^{ΔIEC} mice (FIGS. 35C-35D). Furthermore, the expression of the regulators of proliferation, cell-cycle progression (c-Myc, Cyclin D1, Cyclin D2), and apoptosis (Bax, Bak, Hsp70, Bcl-2 and Bcl-xL), which are downstream targets of Stat3 and polyamines pathway, were examined. It was found that the expression of c-Myc, cyclin D1 and Cyclin D2 was decreased in APC^{Min/+}CSN8^{ΔIEC} mice than in APC^{Min/+}CSN8^{fl/fl} mice (FIG. 35E). Analysis of other downstream targets of Stat3, revealed that the proapoptotic protein Bak and Bax and was significantly upregulated in APC^{Min/+}CSN8^{ΔIEC} mice, whereas the antiapoptotic proteins Bcl-2, and Bcl-XL were expressed at similar levels in APC^{Min/+}CSN8^{fl/fl} and APC^{Min/+}CSN8^{ΔIEC} mice (FIG. 35F). Indeed, an increased expression levels of the cyclin-dependent kinase (Cdk) inhibitor p21^{(Cip1/WAF1)}, a protein that governs cell-cycle arrest and differentiation, was found in the crypts of zileum from APC^{Min/+}CSN8^{ΔIEC} mice. By comparison, p21^{(Cip1/WAF1)} expression was similar in colon between APC^{Min/+}CSN8^{fl/fl} mice and APC^{Min/+}CSN8^{ΔIEC} mice (FIG. 35F). These results correlated well with the expression of Ki-67, suggesting that decreased Stat3 activity leads to hypoproliferation of CSN8-deficient epithelial cells. Collectively, these data show that knockout CSN8 in gut epithelial cells leads to reduction of the polyamine mediated metabolic pathways.

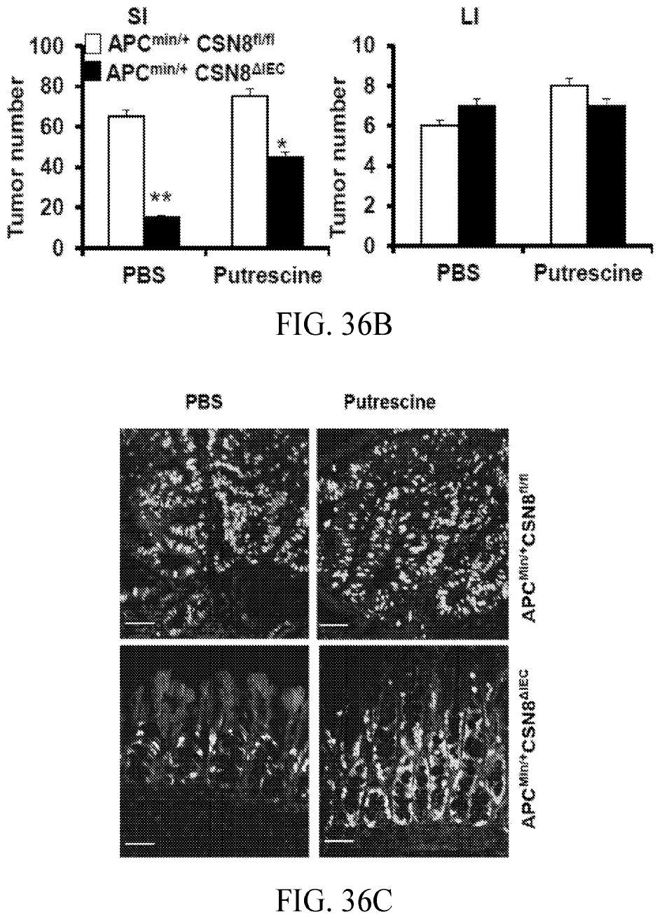

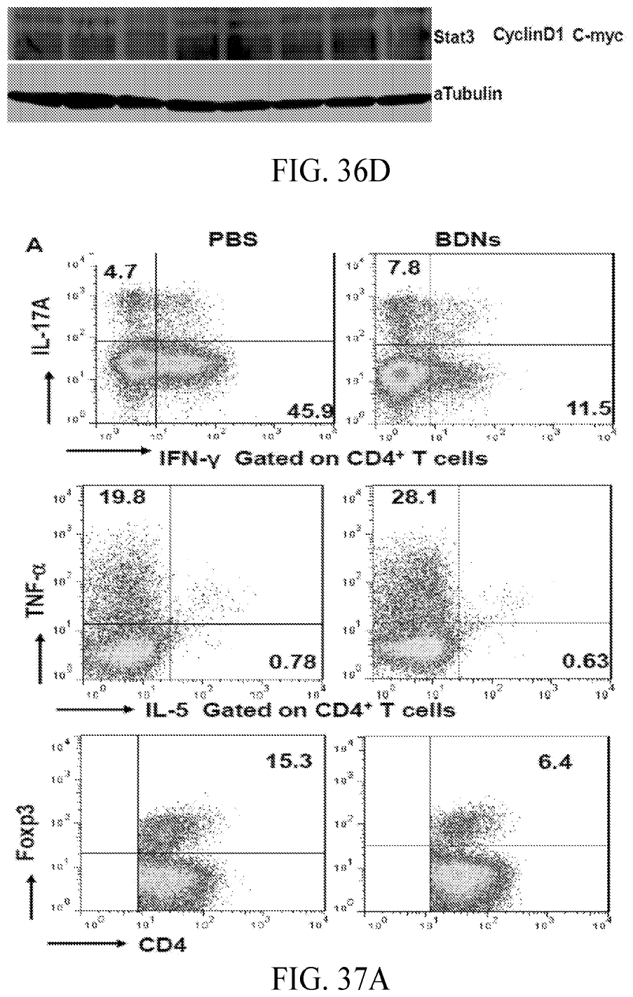

Finally, to test whether polyamines directly affects polyp formation in APC^{Min/+}CSN8^{ΔIEC} mice, mice were supplied with a 1% putrescine solution in the drinking water. Feeding with putrescine led to a large increase in polyamines pool in the small intestine (FIG. 36A). Putrescine-fed APC^{Min/+}CSN8^{fl/fl} mice exhibited an increase, but not statistically significant increase in tumor number compared with control mice. However, feeding with putrescine strikingly increased the polyp numbers in the small intestines of APC^{Min/+}CSN8^{ΔIEC} (FIG. 36B). Putrescine also stimulated epithelial cell proliferation (FIG. 36C) in the small intestines of APC^{Min/+}CSN8^{ΔIEC}. However, putrescine had no effect on polyp number (FIG. 36B) in the colon. Polyamines diet restored the expression of nuclear Stat3, c-Myc and p21^{(Cip1/WAF1)} in APC^{Min/+}CSN8^{ΔIEC} mice to that seen in APC^{Min/+}CSN8^{fl/fl} mice (FIG. 36D). Therefore, the delay in tumor development was associated with specific differences in polyamines pool and the expression of polyamine-related cell cycle markers. Taken together, these data indicated that polyamine metabolites regulated by CSN8 drive cell proliferation and CRC development in APC^{Min/+} mice. Although it is appreciated that both COP9 and polyamine mediated metabolic pathways regulate cell apoptosis, proliferation, and cellular DNA damage, the finding in this study indicated that CSN8 regulate genes associated with polyamine mediated metabolic pathways.

### Example 12—SFN-Rich BDNs Suppress Tumor Growth Through the Reduction of Intestinal Polyamines Levels, and Reduction of Gut Inflammation Via Restoring the Homeostasis of Gut Microbiota

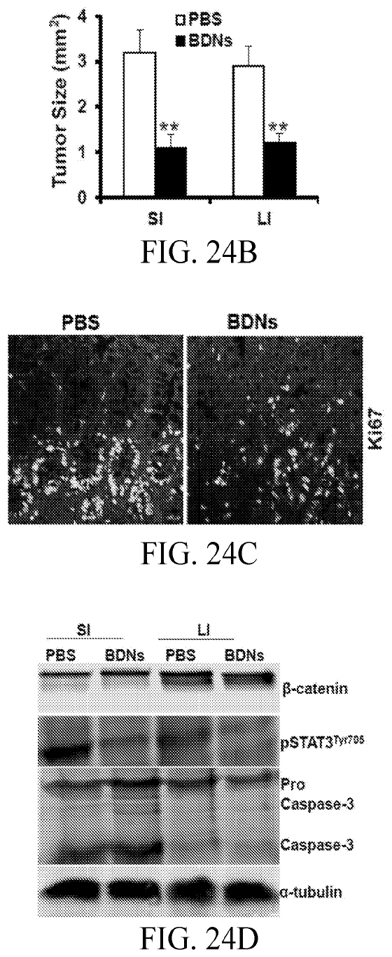

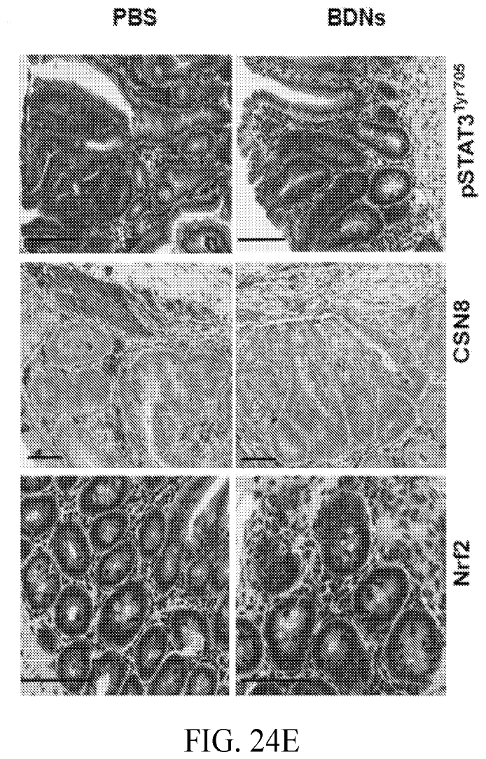

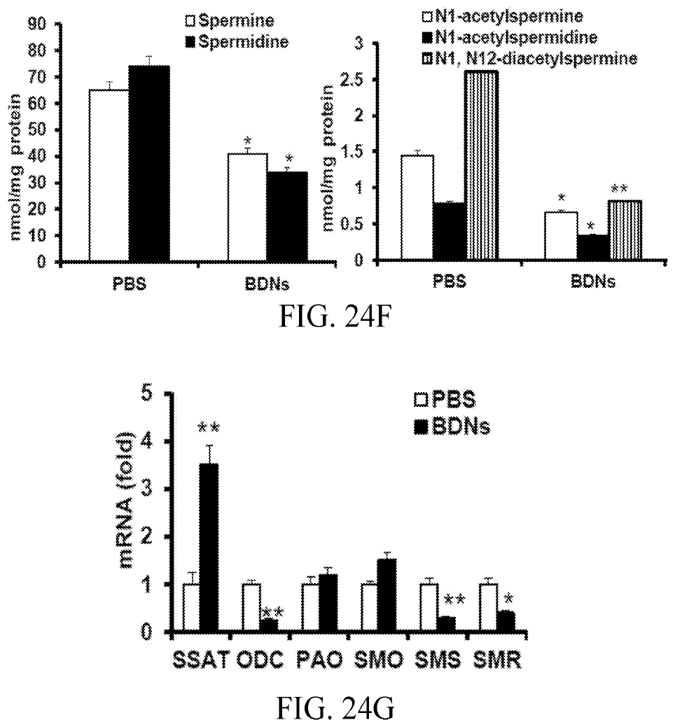

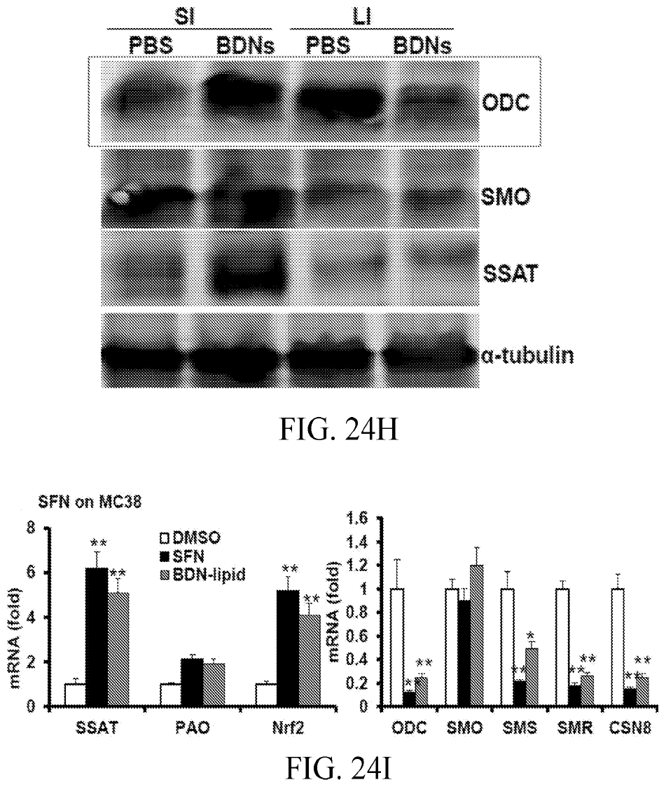

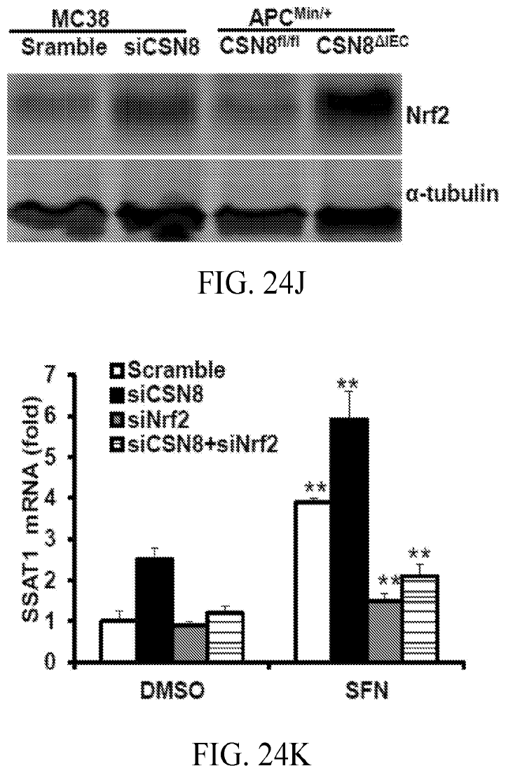

Next, we hypothesize that inhibition of colon tumor growth by blocking induction of polyamine would lead to better strategy for treatment of colon cancer. We recently reported sulforaphane-rich nanoparticles derived from broccoli (SFN-BDNs) suppress the development of colitis when administered orally to mice, but it was not known if SFN-rich BDNs inhibit the polyamine-regulated tumor growth. Five-week-old APC^{Min/+} mice were given PBS or BDNs for 10 weeks. Strikingly, the treatment of BDNs was associated with a significant reduction in polyp number compared with control mice in the small intestine and colon (FIG. 24A). More importantly, the intestinal and colonic polyps that developed in BDNs-treated mice were significantly smaller than those in PBS-treated mice (FIG. 24B). These findings demonstrate that oral delivery of BDNs reduce polyp number and size in an APC^{Min/+} mouse model. Ki67 staining revealed decreased proliferation of epithelial cells in tumors from the ileum of APC^{Min/+} mice after BDNs treatment (FIG. 24C). An increase in apoptosis was also observed in ileum epithelial cells from BDNs-treated APC^{Min/+} mice (FIG. 24D). Importantly, expression levels of activated STAT3 were reduced, in the small intestine of in APC^{Min/+} mice treated with BDNs compared with mice treated with vehicle (FIGS. 24D-24E). Ileum polyamine levels in APC^{Min/+} mice treated with BDNs were next evaluated. It was found that polyamines levels were reduced after 12 weeks of BDNs feeding (FIG. 24F). Western blot and qPCR analysis indicated that that polyamine metabolic pathway in the ileum was decreased, as revealed by a significant increase in the expression of SSAT and a decrease in the expression of ODC (FIGS. 24G-24H). Conversely, BDNs treatment didn't affect the activity of SMO in the ileum (FIG. 24H). Next, it was investigated whether the BDNs mediated inhibition of intestinal and colonic polyps in APC^{Min/+} mice was through the CSN8 regulated polyamine metabolic pathway. SSAT1 has been reported to be transcriptionally regulated through the interaction of two trans-acting transcription factors, nuclear factor erythroid-derived 2 (NF-E2)-related factor 2 (Nrf2) and polyamine modulated factor-1 (PMF-1). It was also recently demonstrated that the COP9 complex appears to play a role in the stability of Nrf2 via regulation of the Cul3-Keap1-E3 ligase and COP9 has been reported to interact with PMF-1. Sulforaphane is known to be a potent Nrf2 activator. Indeed, SFN-rich BDNs supplements strongly induce the expression of Nrf2 and suppress the expression of CSN8 in the ileum of in APC^{min/+} mice (FIGS. 24D-24E). The effect of SFN or BDN-derived lipids on the expression of Nrf2 and CSN8 in colon cancer cells was further measured. Treatment with BDN-derived lipids or SFN strongly activated the expression of Nrf2 and SSAT1 and inhibited the expression of CSN8 and ODC in MC38 colon cancer cells (FIG. 24I). Indeed, deletion of CSN8 did increase the Nrf2 protein level in APC^{min/+} mice and MC38 cells and (FIG. 24J). Moreover, knockdown of both Nrf2 and CSN8 significantly inhibited the increase in SSAT1 caused by SFN (FIG. 24K), indicating that Nrf2 is a downstream of CSN8 to regulate SFN induced SSAT1. These results indicated that SFN treatments mainly suppress tumor growth by decreasing polyamines levels through CSN8-Nrf2 pathway.

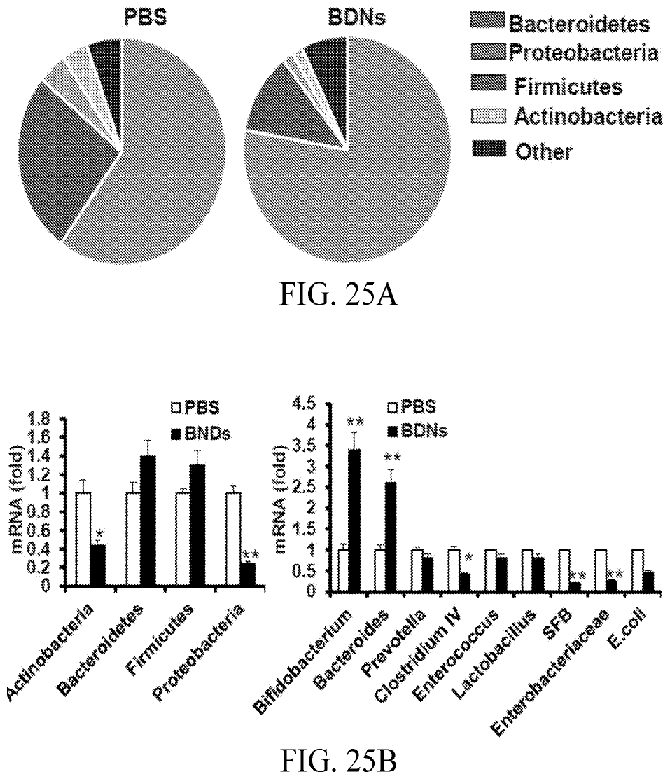

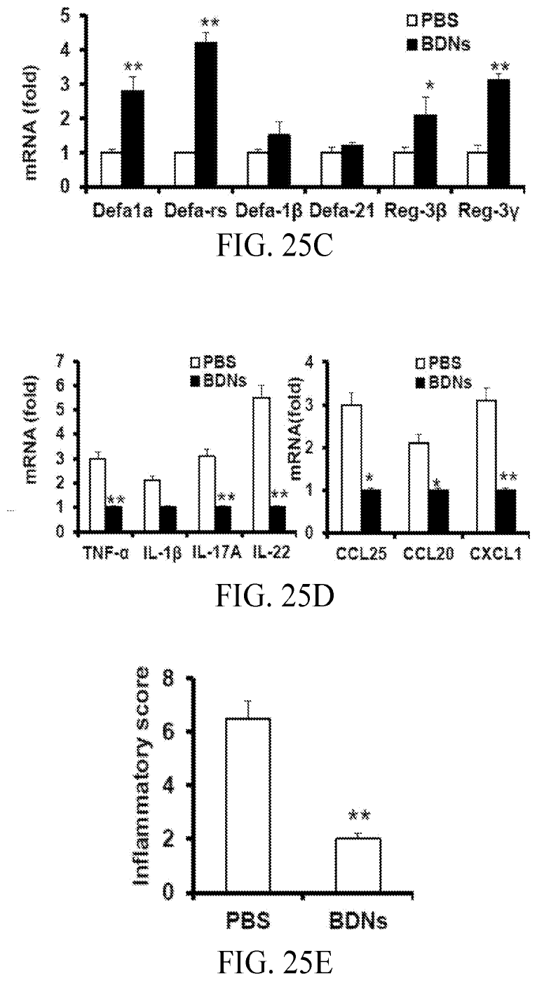

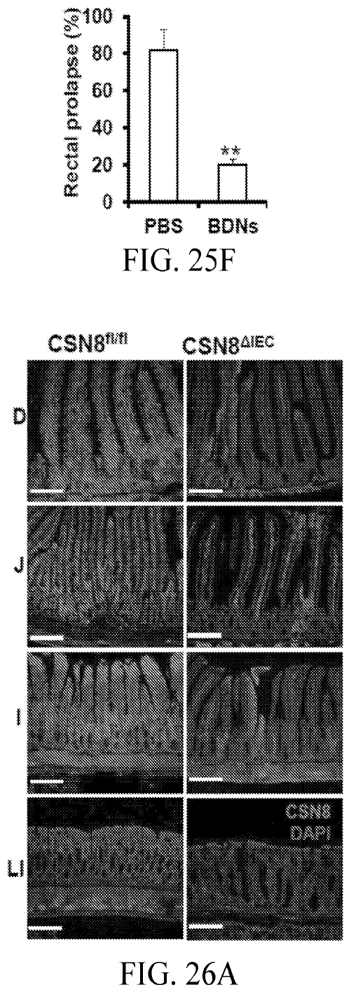

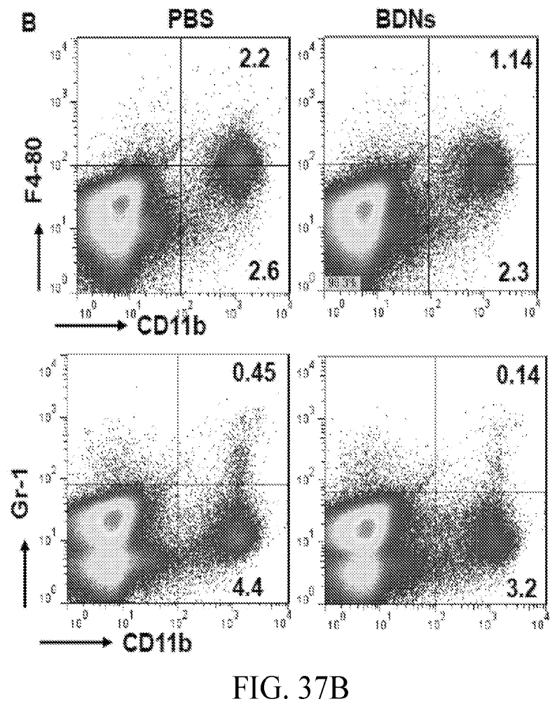

Next, it was investigated whether BDN has an effect of gut microbiota. Although BDN had no effect on total bacterial abundance analyzed by 16S rRNA gene sequencing, significant phylum-level shifts from Firmicutes to Bacteroidetes in the gut microbiome composition (FIG. 25A). RT-PCR analysis showed that BDNs treatment also dramatically decreases the family Actinobacteria and robustly reduces the endotoxin producing Proteobacteria (FIG. 25A-25B). In addition, the levels of the Bifidobacterium and Bacteroides were also substantially increased in BDNs-treated mice, whereas the levels of the Firmicutes remained similar (FIG. 25B). BDNs treatment also led to the induction of antimicrobial peptides in intestinal epithelial cells from APC^{Min/+} mice (FIG. 25C). The impact of BDNs on cytokines in APC^{Min/+} mice was next evaluated. BDNs treatment suppressed cytokine levels (including TNF-α, IL-17A, and IL-22) and chemokine levels (CCL20, CXCL1 and CCL25) compared with the vehicle treatment (FIG. 25D). Strikingly, the BDNs treatment reduced inflammation (FIG. 25E) and the infiltration of immune cells (FIG. 37) in the small intestines and colons of APC^{Min/+} mice and APC^{Min/+}CSN8^{ΔIEC} mice. Of note, BDNs treatment also resulted in a significant decrease of rectal prolapse in APC^{Min/+}CSN8^{ΔIEC} mice (FIG. 25F).

### Discussion of Examples 8-12

Central to the development of cancer are genetic changes that endow these cancer cells with many of the hallmarks of cancer including limitless replicative potential (dysregulated cell cycle) and chronic inflammation in tumor microenvironment. Whether blocking expression of genes that have a role in regulation of a number of processes that are relevant to cancer development and progression, which is one of major therapeutic strategies for chemotherapy, has effect on the level of inflammation which is the major side-effect due to chemotherapy had not been fully investigated. COP9 signalosome (CSN) plays a significant role in the regulation of multiple cancers under sterile environment through regulation of genes that play a role in cell cycle. The role of epithelial COP9 under non-sterile environment with gut enriched microbiota instructions for its intestinal tumor growth was unknown.

In the foregoing study, it was demonstrated that COP9 CSN8 plays a key role in promoting colon tumor development via oncogenic polyamine metabolism pathway and in regulating the expression of an array of genes encoded for antimicrobial peptides secreted by Paneth cells. Utilizing multiple animal models, and gut epithelial specific knockout CSN8, the studies described herein made the findings that: 1) knockout of CSN8 inhibits colon tumor development but induces gut inflammation; 2) gut inflammation induced by knockout (KO) of CSN8 does not promote tumor growth; 3) CSN8 promotes intestinal tumor development via oncogenic polyamine metabolism pathway; 4) as a result of CSN8 KO, significant reduction of an array of genes encoded for antimicrobial peptides leads to intestinal inflammation accompanied with altered fecal gut microbiota composition; and 5) most of chemo drugs on the market target to cell cycle related genes and cause toxicity. The findings suggest that therapeutic targeting those genes such as COP9 CSN8 that play a role in the regulation of cell cycle may cause toxicity such as chronic inflammation. As such, and alternative approach can be to take edible nanoparticles, such as the broccoli nanoparticles as described in the present study to inhibit the oncogenic polyamine metabolism pathway as well as restore gut microbiome homeostasis.

Collectively, the data generated in the studies described herein indicate that the intestinal tumorigenesis pathways) regulated by COP9 CSN8 is separated from the inflammation pathway in microbiota enriched intestinal environment for its intestinal tumor development and growth. This pro-intestinal tumor developmental environment can be turned into anti-tumor developmental environment by giving healthy diet derived nanoparticles such as broccoli nanoparticles. Nanoparticles from diet can modulate the intestinal microenvironment. Since edible nanoparticles are present in the different types of diet, this finding provides a foundation for selecting personalized edible nanoparticles for chemoprevention with no or minimal off-target effects. The findings should also provide a rationale for further studying the mechanisms underlying how nanoparticles from diet cross-talk with gut microbiota to modulate the multiple steps of tumor development.

COP9 signalosome interacts with multiple signaling molecules. COP9 signalosome is an evolutionarily conserved multi-protein complex involved in tumorigenesis, signal transduction, cell cycle, and transcriptional activation. CSN8, the one subunit of COP9, is required for T cell homeostasis and normal postnatal cardiac development. The detailed biological functions of CSN8 remain largely unclear. Here, we reveal that gut epithelial cell CSN8 has role in the promoting colon tumorigenesis through the specific upregulation of polyamine mediated pathway and inhibiting gut inflammation through regulation of expression of antimicrobial peptides. This conclusion is supported by the fact that gut epithelial cell specific knockout of CSN8 led to inhibition of tumor development and spontaneously inflammation induced.

The foregoing data indicate that CSN8 KO leads to the reduction of expression of lysozyme, which is a marker of Paneth cells and induced chronic intestinal inflammation. Paneth cells are secretory cells in the epithelium of the small intestine and large secretory granules in these cells contain antimicrobials. The antimicrobial-rich granules are discharged into the crypt lumen and prevent microbial invasion of the crypt. Here, it was found that Paneth cell CSN8 is an essential factor for maintaining homeostasis of lysozyme⁺ Paneth cells and expression of antimicrobials. Knockout of CSN8 in gut epithelial leads to dysregulation of gut microbiome, which cause gut chronic inflammation. Surprisingly, gut inflammatory microenvironment induced by knockout CSN8 does not contribute to tumor development in both and APC^{Min/+} and AOM plus DSS induced mouse colon cancer models demonstrated in this study.

A number of studies highlight the role of microbes to elicit their oncogenic effects through induction of inflammation. However, polyp incidence is not reduced in germ-free APC^{Min/+} mice, and the etiology of most mouse models does not have an inflammatory component at the initiation stage during tumor development. Furthermore, although identification of composition of microbial changes is associated with colon cancer development, the changes is not sufficient to fully understand the role of the microbiota in health and disease since many factors such as healthy diet also cause the composition of changes of gut microbiome. Therefore, a question is whether the inflammation induced due to composition of changes of these microorganisms actively drive the process of carcinogenesis has not been clearly addressed. Appropriate dissection of the different gut inflammatory microenvironment induced as a result from the interaction of tumor cells derived factors) such as CSN8 with microbial community is still in its infancy, and largely unknown. The above findings suggest that gut inflammatory microenvironment provided the “CSN8 defect soil” is not suitable for pro-tumor development.

On the other hand, the foregoing data demonstrate that CSN8 has a property in promotion of tumor development through regulation of activity of polyamine mediated metabolic pathway. The metabolism of polyamine is frequently dysregulated in neoplastic disease. Although the requirement for polyamines in cell growth is recognized, CSN8 mediated pathway that regulates the activity of the metabolism of polyamines has not been investigated. Here, it was discovered that CSN8 regulated the metabolism of polyamines. The results demonstrate the knockout of CSN8 reduces activity of polyamines metabolic pathways, providing a foundation for developing strategies for targeting CSN8 mediated downstream of tumorigenic pathway and preventing colon cancer development. However, knockout of CSN8 also induces gut inflammation, which is not desirable for therapeutic application.

In human populations, epidemiological studies have shown that environmental factors, especially diet, plays an important role in colon cancer susceptibility. It would be ideal to have healthy diet derived agent that can down-regulate the expression of CSN8 as well as inhibition of gut inflammation for treatment or prevention of colon cancer. In the present study, this feature was examined in APC^{Min/+} mice by placing them on broccoli exosomes-like nanoparticles educated gut microenvironment. The data show that mice fed with broccoli-derived nanoparticles had reduced incidence of polyp formation in both small and large intestine. The molecular mechanism underlying this phenotype was further provided by the data. BDN is enriched with sulforaphane and has anti-inflammatory effect. Sulforaphane is known to be a potent Nrf2 activator and Nrf2 has been reported to suppress activity of polyamines metabolic pathways. However, whether BDN enriched with sulforaphane can inhibit activity of polyamines metabolic pathways was not known. In this study, the results generated from both in vitro and in vivo mouse models indicated that treatment with sulforaphane enriched BDN lipids or SFN strongly activates the expression of Nrf2 and inhibits the expression of CSN8. Polyamines levels in the gut epithelium were reduced and APC^{Min/+} mice were treated with BDNs for 12 weeks. These results agreed with a significant reduction in polyp number and size in the small intestine and colon of and APC^{Min/+} mice treated with BDNs.

Recent findings have suggested possible links between polyamine catabolism and tumor development. In particular, the polyamine catabolic enzymes spermine oxidase (SMO), spermidine/spermine N1-acetyltransferase (SSAT), and N1-acetylpolyamine oxidase (APAO) are considered as potential sources of oxidative stress capable of damaging crucial cellular machinery or potentially contributing to oxidative DNA damage or chromatin instability. Therefore, SMO and SSAT provide new targets for chemoprevention and/or chemotherapy. However, polyamines, including spermine, are required for eukaryotic cell growth, differentiation, and survival. This absolute requirement for polyamines and the need to maintain intracellular levels within specific ranges require a highly regulated metabolic pathway primed for rapid changes in response to cellular growth signals, environmental changes, and stress. Therefore, unregulated inhibition of activity of polyamines by giving sulforaphane may cause unpredicted side effects. The present finding that food derived nanoparticles, such as BDNs with enriched sulforaphane, regulate the activity of polyamine catabolism pathway will open a new avenue for treatment of cancer due to dysregulated polyamine catabolism pathways.

BDNs treatment also resulted in a significant decrease of rectal prolapse in APC^{Min/+}CSN8^{ΔIEC} mice and restored gut microbiota homeostasis. Collectively, BDN was used as an example to show that gut microenvironment can be altered from pro- to anti-tumor development as well as inhibition of gut inflammation. Exosomes-like nanoparticles (ELNs) are present in a number of edible plants, perhaps all different types of food we daily eat, and the composition of one type of ELNs is different from others. Therefore, personalized and healthier gut microenvironment can be created by taking customized ELNs. Therefore, the results generated from this study provide a foundation for selecting use of edible exosomes-like nanoparticles based on individual needs for prevention/treatment disease by targeting gut microbiota and epithelial cells for restoring gut homeostasis. Although there is great promise in chemoprevention, the major impediment is identifying appropriate targets and then developing agents that can be safely administered over the lifetime of an individual with no or minimal off-target effects. ELNs from food we daily eat, not only do we not need to consider safety, but each type of ELNs which have unique molecular profiles and preferential targets. Therefore, ELNs could be developed as a safe and targetable chemoprevention agent.

All publications, patents, and patent applications mentioned in this specification are herein incorporated by reference, including the references set forth in the following list:

### REFERENCES

1. Rossi, M. & Bot, A. The Th17 cell population and the immune homeostasis of the gastrointestinal tract. International reviews of immunology 32, 471-474, doi: 10.3109/08830185.2013.843983 (2013).
2. Shan, M. et al. Mucus enhances gut homeostasis and oral tolerance by delivering immunoregulatory signals. Science 342, 447-453, doi: 10.1126/science. 1237910 (2013).
3. Magrone, T. & Jirillo, E. The interplay between the gut immune system and microbiota in health and disease: nutraceutical intervention for restoring intestinal homeostasis. Current pharmaceutical design 19, 1329-1342 (2013).
4. Devkota, S. et al. Dietary-fat-induced taurocholic acid promotes pathobiont expansion and colitis in I110−/− mice. Nature 487, 104-108, doi:10.1038/nature11225 (2012).
5. Jyonouchi, H. Non-IgE mediated food allergy-update of recent progress in mucosal immunity. Inflammation & allergy drug targets 11, 382-396 (2012).
6. Kim, K. S. et al. Dietary antigens limit mucosal immunity by inducing regulatory T cells in the small intestine. Science 351, 858-863, doi:10.1126/science.aac5560 (2016).
7. Shan, M. M. et al. Mucus Enhances Gut Homeostasis and Oral Tolerance by Delivering Immunoregulatory Signals. Science 342, 447-453, doi: 10.1126/science. 1237910 (2013).
8. Rivas, M. N. et al. A microbiota signature associated with experimental food allergy promotes allergic sensitization and anaphylaxis. J Allergy Clin Immun 131, 201-212, doi:10.1016/j.jaci.2012.10.026 (2013).
9. Leonard, S. A., Martos, G., Wang, W., Nowak-Wegrzyn, A. & Berin, M. C. Oral immunotherapy induces local protective mechanisms in the gastrointestinal mucosa. J Allergy Clin Immun 129, 1579-+, doi:10.1016/j.jaci.2012.04.009 (2012).
10. Antonioli, L. et al. The AMPK enzyme-complex: from the regulation of cellular energy homeostasis to a possible new molecular target in the management of chronic inflammatory disorders. Expert opinion on therapeutic targets 20, 179-191, doi: 10.1517/14728222.2016.1086752 (2016).
11. Vasamsetti, S. B. et al. Metformin Inhibits Monocyte-to-Macrophage Differentiation via AMPK-Mediated Inhibition of STAT3 Activation: Potential Role in Atherosclerosis. Diabetes 64, 2028-2041, doi:10.2337/db14-1225 (2015).
12. Carroll, K. C., Viollet, B. & Suttles, J. AMPKalpha1 deficiency amplifies proinflammatory myeloid APC activity and CD40 signaling. Journal of leukocyte biology 94, 1113-1121, doi: 10.1189/jlb.0313157 (2013).
13. Ramiscal, R. R. et al. Attenuation of AMPK signaling by ROQUIN promotes T follicular helper cell formation. eLife 4, doi:10.7554/eLife.08698 (2015).
14. Din, F. V. et al. Aspirin inhibits mTOR signaling, activates AMP-activated protein kinase, and induces autophagy in colorectal cancer cells. Gastroenterology 142, 1504-1515 e1503, doi:10.1053/j.gastro.2012.02.050 (2012).
15. Garcin, G. et al. AMPK/HuR-Driven IL-20 Post-Transcriptional Regulation in Psoriatic Skin. The Journal of investigative dermatology 135, 2732-2741, doi:10.1038/jid.2015.282 (2015).
16. Cao, Q. et al. Myeloid deletion of alpha 1 AMPK exacerbates atherosclerosis in low density lipoprotein receptor knockout (LDLRKO) mice. Diabetes, doi:10.2337/db15-0917 (2016).
17. An, Y. W., Jhang, K. A., Woo, S. Y., Kang, J. L. & Chong, Y. H. Sulforaphane exerts its anti-inflammatory effect against amyloid-beta peptide via STAT-1 dephosphorylation and activation of NrG/HO-1 cascade in human THP-1 macrophages. Neurobiology of aging 38, 1-10, doi:10.1016/j.neurobiolaging.2015.10.016 (2016).
18. Bai, Y. et al. Sulforaphane Protects against Cardiovascular Disease via Nrf2 Activation. Oxidative medicine and cellular longevity 2015, 407580, doi:10.1155/2015/407580 (2015).
19. Greaney, A. J., Maier, N. K., Leppla, S. H. & Moayeri, M. Sulforaphane inhibits multiple inflammasomes through an NrG-independent mechanism. Journal of leukocyte biology 99, 189-199, doi:10.1189/jlb.3A0415-155RR (2016).
20. Liu, H. & Talalay, P. Relevance of anti-inflammatory and antioxidant activities of exemestane and synergism with sulforaphane for disease prevention. Proceedings of the National Academy of Sciences of the United States of America 110, 19065-19070, doi:10.1073/pnas.1318247110 (2013).
21. Zhuang, X. et al. Ginger-derived nanoparticles protect against alcohol-induced liver damage. Journal of extracellular vesicles 4, 28713, doi:10.3402/jev.v4.28713 (2015).
22. Mu, J. et al. Interspecies communication between plant and mouse gut host cells through edible plant derived exosome-like nanoparticles. Molecular nutrition & food research 58, 1561-1573, doi: 10.1002/mnfr.201300729 (2014).
23. Ju, S. et al. Grape exosome-like nanoparticles induce intestinal stem cells and protect mice from DSS-induced colitis. Molecular therapy: the journal of the American Society of Gene Therapy 21, 1345-1357, doi:10.1038/mt.2013.64 (2013).
24. Zhang, H. G. et al. Isolation, identification, and characterization of novel nanovesicles. Oncotarget 7, 41346-41362, doi: 10.18632/oncotarget.9325 (2016).
25. Chinen, H. et al. Lamina propria c-kit(+) immune precursors reside in human adult intestine and differentiate into natural killer cells. Gastroenterology 133, 559-573, doi: 10.1053/j.gastro.2007.05.017 (2007).
26. Sakuraba, A. et al. Th1/Th17 Immune Response Is Induced by Mesenteric Lymph Node Dendritic Cells in Crohn's Disease. Gastroenterology 137, 1736-1745, doi: 10.1053/j.gastro.2009.07.049 (2009).
27. Okayasu, I. et al. A novel method in the induction of reliable experimental acute and chronic ulcerative colitis in mice. Gastroenterology 98, 694-702 (1990).
28. Dunay, I. R. et al. Gr1(+) inflammatory monocytes are required for mucosal resistance to the pathogen Toxoplasma gondii. Immunity 29, 306-317, doi: 10.1016/j.immuni.2008.05.019 (2008).
29. Salazar-Gonzalez, R. M. et al. CCR6-mediated dendritic cell activation of pathogen-specific T cells in Foyer's patches. Immunity 24, 623-632, doi: 10.1016/j.immuni.2006.02.015 (2006).
30. Geissmann, F., Jung, S. & Littman, D. R. Blood monocytes consist of two principal subsets with distinct migratory properties. Immunity 19, 71-82 (2003).
31. Uhlig, H. H. et al. Differential activity of IL-12 and IL-23 in mucosal and systemic innate immune pathology. Immunity 25, 309-318, doi:10.1016/j.immuni.2006.05.017 (2006).
32. Wang, B. M. et al. Targeted Drug Delivery to Intestinal Macrophages by Bioactive Nanovesicles Released from Grapefruit. Molecular Therapy 22, 522-534, doi:10.1038/mt.2013.190 (2014).
33. Li, W., Zhang, T., Ye, Y., Zhang, X. & Wu, B. Enhanced bioavailability of tripterine through lipid nanoparticles using broccoli-derived lipids as a carrier material. International journal of pharmaceutics 495, 948-955, doi:10.1016/j.ijpharm.2015.10.011 (2015).
34. Herber, D. L. et al. Lipid accumulation and dendritic cell dysfunction in cancer. Nature medicine 16, 880-886, doi:10.1038/nm.2172 (2010).
35. Worbs, T. et al. Oral tolerance originates in the intestinal immune system and relies on antigen carriage by dendritic cells. J Exp Med 203, 519-527, doi:10.1084/jem.20052016 (2006).
36. Rescigno, M. et al. Dendritic cells express tight junction proteins and penetrate gut epithelial monolayers to sample bacteria. Nat Immunol 2, 361-367, doi: 10.1038/86373 (2001).
37. Maynard, C. L. & Weaver, C. T. Diversity in the contribution of interleukin-10 to T-cell-mediated immune regulation. Immunological reviews 226.219-233, doi:10.1111/j.1600-065X.2008.00711.x (2008).
38. Salt, I. P. & Palmer, T. M. Exploiting the anti-inflammatory effects of AMP-activated protein kinase activation. Expert opinion on investigational drugs 21, 1155-1167, doi:10.1517/13543784.2012.696609(2012).
39. Zhu, Y. P., Brown, J. R., Sag, D., Zhang, L. & Suttles, J. Adenosine 5-monophosphate-activated protein kinase regulates IL-10-mediated anti-inflammatory signaling pathways in macrophages. J Immunol 194, 584-594, doi: 10.4049/jimmunol. 1401024 (2015).
40. Panieri, E. et al. Nutrient withdrawal rescues growth factor-deprived cells from mTOR-dependent damage. Aging 2, 487-503, doi:10.18632/aging.100183 (2010).
41. Sun, C. C. et al. Sulforaphane Attenuates Muscle Inflammation in Dystrophin-deficient mdx Mice via NF-E2-related Factor 2 (Nrf2)-mediated Inhibition of NF-kappaB Signaling Pathway. The Journal of biological chemistry 290, 17784-17795, doi:10.1074/jbc.M115.655019 (2015).
42. Pennisi, M. et al. Inflammasomes, hormesis, and antioxidants in neuroinflammation: Role of NRLP3 in Alzheimer disease. Journal of neuroscience research, doi: 10.1002/jnr.23986 (2016).
43. Fuentes, F., Paredes-Gonzalez, X. & Kong, A. T. Dietary Glucosinolates Sulforaphane, Phenethyl Isothiocyanate, Indole-3-Carbinol/3,3′-Diindolylmethane: Anti-Oxidative Stress/Inflammation, Nrf2, Epigenetics/Epigenomics and In Vivo Cancer Chemopreventive Efficacy. Curr Pharmacol Rep 1, 179-196, doi:10.1007/s40495-015-0017-y (2015).
44. Raimondo, S. et al. Citrus limon-derived nanovesicles inhibit cancer cell proliferation and suppress CML xenograft growth by inducing TRAIL-mediated cell death. Oncotarget 6, 19514-19527 (2015).
45. Ju, S. W. et al. Grape Exosome-like Nanoparticles Induce Intestinal Stem Cells and Protect Mice From DSS-Induced Colitis. Molecular Therapy 21, 1345-1357, doi: 10.1038/mt.2013.64 (2013).
46. Mu, J. Y. et al. Interspecies communication between plant and mouse gut host cells through edible plant derived exosome-like nanoparticles. Mol Nutr Food Res 58, 1561-1573, doi: 10.1002/mnfr.201300729 (2014).
47. Hashimoto, D., Miller, J. & Merad, M. Dendritic cell and macrophage heterogeneity in vivo. Immunity 35, 323-335, doi:10.1016/j.immuni.2011.09.007 (2011).
48. DePaolo, R. W. et al. Co-adjuvant effects of retinoic acid and IL-15 induce inflammatory immunity to dietary antigens. Nature 471, 220-U110, doi:10.1038/nature09849 (2011).
49. Zhang, H. G. et al. Isolation, identification, and characterization of novel nanovesicles. Oncotarget, doi:10.18632/oncotarget.9325 (2016).
50. Jorgensen, S. B. et al. Knockout of the alpha(2) but not alpha(1) 5′-AMP-activated protein kinase isoform abolishes 5-aminoimidazole-4-carboxamide-1-beta-4-ribofuranoside—but not contraction—induced glucose uptake in skeletal muscle. J Biol Chem 279, 1070-1079, doi:10.1074/jbc.M306205200 (2004).
51. Viollet, B. et al. The AMP-activated protein kinase alpha 2 catalytic subunit controls whole-body insulin sensitivity. J Clin Invest 111, 91-98, doi: 10.1172/Jci200316567 (2003).
52. Deng, Z. B. et al. Enterobacteria-secreted particles induce production of exosome-like SIP-containing particles by intestinal epithelium to drive Th17-mediated tumorigenesis. Nat Commun 6, doi:Artn 695610.1038/Ncomms7956 (2015).
53. Cooper, H. S., Murthy, S. N., Shah, R. S. & Sedergran, D. J. Clinicopathologic study of dextran sulfate sodium experimental murine colitis. Lab Invest 69, 238-249 (1993).
54. Uhlig, H. H. et al. Differential activity of IL-12 and IL-23 in mucosal and systemic innate immune pathology. Immunity 25, 309-318, doi:10.1016/j.immuni.2006.05.017 (2006).
55. Dieleman, L. A., Pena, A. S., Meuwissen, S. G. & van Rees, E. P. Role of animal models for the pathogenesis and treatment of inflammatory bowel disease. Scandinavian journal of gastroenterology. Supplement 223, 99-104 (1997).
56. Read, S., Malmstrom, V. & Powrie, F. Cytotoxic T lymphocyte-associated antigen 4 plays an essential role in the function of CD25(+)CD4(+) regulatory cells that control intestinal inflammation. J Exp Med 192, 295-302 (2000).
57. Xiang, Y. et al. MiR-152 and miR-185 co-contribute to ovarian cancer cells cisplatin sensitivity by targeting DNMT1 directly: a novel epigenetic therapy independent of decitabine. Oncogene 33, 378-386, doi:10.1038/onc.2012.575 (2014).
58. Wang, Q. et al. Grapefruit-Derived Nanovectors Use an Activated Leukocyte Trafficking Pathway to Deliver Therapeutic Agents to Inflammatory Tumor Sites. Cancer research 75, 2520-2529, doi:10.1158/0008-5472.CAN-14-3095 (2015).
59. Bligh, E. G. & Dyer, W. J. A rapid method of total lipid extraction and purification. Canadian journal of biochemistry and physiology 37, 911-917 (1959).
60. Torrelles, J. B. et al. Isolation of a distinct Mycobacterium tuberculosis mannose-capped lipoarabinomannan isoform responsible for recognition by CD1b-restricted T cells. Glycobiology 22, 1118-1127, doi:10.1093/glycob/cws078 (2012).
61. Wang, B. et al. Targeted drug delivery to intestinal macrophages by bioactive nanovesicles released from grapefruit. Molecular therapy: the journal of the American Society of Gene Therapy 22, 522-534, doi:10.1038/mt.2013.190 (2014).
62. Adler, A. S., Littlepage, L. E., Lin, M., Kawahara, T. L., Wong, D. J., Werb, Z., and Chang, H. Y. (2008). CSN5 isopeptidase activity links COP9 signalosome activation to breast cancer progression. Cancer Res 68, 506-515.
63. Alenghat, T., Osborne, L. C., Saenz, S. A., Kobuley, D., Ziegler, C. G. K., Mullican, S. E., Choi, I., Grunberg, S., Sinha, R., Wynosky-Dolfi, M., et al. (2013). Histone deacetylase 3 coordinates commensal-bacteria-dependent intestinal homeostasis. Nature 504, 153-157.
64. Arthur, J. C., Perez-Chanona, E., Muhlbauer, M., Tomkovich, S., Uronis, J. M., Fan, T. J., Campbell, B. J., Abujamel, T., Dogan, B., Rogers, A. B., et al. (2012). Intestinal inflammation targets cancer-inducing activity of the microbiota. Science 338, 120-123.
65. Axelsson, A. S., Tubbs, E., Mecham, B., Chacko, S., Nenonen, H. A., Tang, Y. Z., Fahey, J. W., Derry, J. M. J., Wollheim, C. B., Wierup, N., et al. (2017). Sulforaphane reduces hepatic glucose production and improves glucose control in patients with type 2 diabetes. Sci Transl Med 9.
66. Ayabe, T., Satchell, D. P., Wilson, C. L., Parks, W. C., Selsted, M. E., and Ouellette, A. J. (2000). Secretion of microbicidal alpha-defensins by intestinal Paneth cells in response to bacteria. Nat Immunol 1, 113-118.
67. Barker, N. (2014). Adult intestinal stem cells: critical drivers of epithelial homeostasis and regeneration. Nat Rev Mol Cell Biol 15, 19-33.
68. Battaglia, V., DeStefano Shields, C., Murray-Stewart, T., and Casero, R. A., Jr. (2014). Polyamine catabolism in carcinogenesis: potential targets for chemotherapy and chemoprevention. Amino Acids 46, 511-519.
69. Belcheva, A., Irrazabal, T., Robertson, S. J., Streutker, C., Maughan, H., Rubino, S., Moriyama, E. H., Copeland, J. K., Surendra, A., Kumar, S., et al. (2014). Gut microbial metabolism drives transformation of MSH2-deficient colon epithelial cells. Cell 158, 288-299.
70. Bevins, C. L., and Salzman, N. H. (2011). Paneth cells, antimicrobial peptides and maintenance of intestinal homeostasis. Nat Rev Microbiol 9, 356-368.
71. Bollrath, J., Phesse, T. J., von Burstin, V. A., Putoczki, T., Bennecke, M., Bateman, T., Nebelsiek, T., Lundgren-May, T., Canli, O., Schwitalla, S., et al. (2009). gp130-mediated Stat3 activation in enterocytes regulates cell survival and cell-cycle progression during colitis-associated tumorigenesis. Cancer Cell 15, 91-102.
72. Chen, B., Zhao, R., Su, C. H., Linan, M., Tseng, C., Phan, L., Fang, L., Yang, H. Y., Yang, H., Wang, W., et al. (2012). CDK inhibitor p57 (Kip2) is negatively regulated by COP9 signalosome subunit 6. Cell Cycle 11, 4633-4641.
73. Chen, J., Shin, J. H., Zhao, R., Phan, L., Wang, H., Xue, Y., Post, S. M., Ho Choi, H., Chen, J. S., Wang, E., et al. (2014). CSN6 drives carcinogenesis by positively regulating Myc stability. Nat Commun 5, 5384.
74. Deng, Z., Pardi, R., Cheadle, W., Xiang, X., Zhang, S., Shah, S. V., Grizzle, W., Miller, D., Mountz, J., and Zhang, H. G. (2011). Plant homologue constitutive photomorphogenesis 9 (COP9) signalosome subunit CSN5 regulates innate immune responses in macrophages. Blood 117, 4796-4804.
75. Deng, Z., Rong, Y., Teng, Y., Mu, J., Zhuang, X., Tseng, M., Samykutty, A., Zhang, L., Yan, J., Miller, D., et al. (2017). Broccoli-Derived Nanoparticle Inhibits Mouse Colitis by Activating Dendritic Cell AMP-Activated Protein Kinase. Mol Ther 25, 1641-1654.
76. Dove, W. F., Clipson, L., Gould, K. A., Luongo, C., Marshall, D. J., Moser, A. R., Newton, M. A., and Jacoby, R. F. (1997). Intestinal neoplasia in the Apc(Min) mouse: Independence from the microbial and natural killer (beige locus) status. Cancer Res 57, 812-814.
77. Fang, L., Lu, W., Choi, H. H., Yeung, S. C., Tung, J. Y., Hsiao, C. D., Fuentes-Mattei, E., Menter, D., Chen, C., Wang, L., et al. (2015). ERK2-Dependent Phosphorylation of CSN6 Is Critical in Colorectal Cancer Development. Cancer Cell 28, 183-197.
78. Furuya, A. K. M., Sharifi, H. J., Jellinger, R. M., Cristofano, P., Shi, B. S., and de Noronha, C. M. C. (2016). Sulforaphane Inhibits HIV Infection of Macrophages through Nrf2. Plos Pathog 12.
79. Gemer, E. W., and Meyskens, F. L., Jr. (2004). Polyamines and cancer: old molecules, new understanding. Nat Rev Cancer 4, 781-792.
80. Grivennikov, S., Karin, E., Terzic, J., Mucida, D., Yu, G. Y., Vallabhapurapu, S., Scheller, J., Rose-John, S., Cheroutre, H., Eckmann, L., and Karin, M. (2009). IL-6 and Stat3 are required for survival of intestinal epithelial cells and development of colitis-associated cancer. Cancer Cell 75, 103-113.
81. Grivennikov, S. I., Wang, K., Mucida, D., Stewart, C. A., Schnabl, B., Jauch, D., Taniguchi, K., Yu, G. Y., Osterreicher, C. H., Hung, K. E., et al. (2012). Adenoma-linked barrier defects and microbial products drive IL-23/IL-17-mediated tumour growth. Nature 491, 254-258.
82. Hashimoto, T., Perlot, T., Rehman, A., Trichereau, J., Ishiguro, H., Paolino, M., Sigl, V., Hanada, T., Hanada, R., Lipinski, S., et al. (2012). ACE2 links amino acid malnutrition to microbial ecology and intestinal inflammation. Nature 487, 477-481.
83. Hu, X., Washington, S., Verderame, M. F., and Manni, A. (2005). Interaction between Polyamines and the Mitogen-Activated Protein Kinase Pathway in the Regulation of Cell Cycle Variables in Breast Cancer Cells. Cancer Res 65, 11026-11033.
84. Huber, S., Gagliani, N., Zenewicz, L. A., Huber, F. J., Bosurgi, L., Hu, B., Hedl, M., Zhang, W., O'Connor, W., Murphy, A. J., et al. (2012). IL-22BP is regulated by the inflammasome and modulates tumorigenesis in the intestine. Nature 491, 259-+.
85. Ignatenko, N. A., Besselsen, D. G., Roy, U. K., Stringer, D. E., Blohm-Mangone, K. A., Padilla-Torres, J. L., Guillen, R. J., and Gemer, E. W. (2006). Dietary putrescine reduces the intestinal anticarcinogenic activity of sulindac in a murine model of familial adenomatous polyposis. Nutr Cancer 56, 172-181.
86. Kramer, D. L., Chang, B. D., Chen, Y., Diegelman, P., Alm, K., Black, A. R., Roninson, I. B., and Porter, C. W. (2001). Polyamine depletion in human melanoma cells leads to G1 arrest associated with induction of p21WAF1/CIP1/SDI1, changes in the expression of p21-regulated genes, and a senescence-like phenotype. Cancer Res 61, 7754-7762.
87. Kwak, M. K., Kensler, T. W., and Casero, R. A. (2003). Induction of phase 2 enzymes by serum oxidized polyamines through activation of Nrf2: effect of the polyamine metabolite acrolein. Biochem Bioph Res Co 305, 662-670.
88. Kwok, S. F., Solano, R., Tsuge, T., Chamovitz, D. A., Ecker, J. R., Matsui, M., and Deng, X. W. (1998). Arabidopsis homologs of a c-Jun coactivator are present both in monomeric form and in the COP9 complex, and their abundance is differentially affected by the pleiotropic cop/det/fus mutations. Plant Cell 10, 1779-1790.
89. Lei, D., Li, F., Su, H., Tian, Z., Ye, B., Wei, N., and Wang, X. (2011). COP9 signalosome subunit 8 is required for postnatal hepatocyte survival and effective proliferation. Cell Death Differ 18, 259-270.
90. Lim, S. O., Li, C. W., Xia, W., Cha, J. H., Chan, L. C., Wu, Y., Chang, S. S., Lin, W. C., Hsu, J. M., Hsu, Y. H., et al. (2016). Deubiquitination and Stabilization of PD-L1 by CSN5. Cancer Cell 30, 925-939.
91. Liu, C., Guo, L.-Q., Menon, S., Jin, D., Pick, E., Wang, X., Deng, X. W., and Wei, N. (2013). COP9 Signalosome Subunit Csn8 Is Involved in Maintaining Proper Duration of the G1 Phase. Journal of Biological Chemistry 288, 20443-20452.
92. Liu, L., Li, L., Rao, J. N., Zou, T., Zhang, H. M., Boneva, D., Bernard, M. S., and Wang, J. Y. (2005). Polyamine-modulated expression of c-myc plays a critical role in stimulation of normal intestinal epithelial cell proliferation. Am J Physiol Cell Physiol 288, C89-99.
93. Lyapina, S., Cope, G., Shevchenko, A., Serino, G., Tsuge, T., Zhou, C., Wolf, D. A., Wei, N., Shevchenko, A., and Deshaies, R. J. (2001). Promotion of NEDD-CUL1 conjugate cleavage by COP9 signalosome. Science 292, 1382-1385.
94. Matsuoka, K., and Kanai, T. (2015). The gut microbiota and inflammatory bowel disease.

Semin Immunopathol 37, 47-55.
95. Meir, M., Galanty, Y., Kashani, L., Blank, M., Khosravi, R., Femandez-Avila, M. J., Cruz-Garcia, A., Star, A., Shochot, L., Thomas, Y., et al. (2015). The COP9 signalosome is vital for timely repair of DNA double-strand breaks. Nucleic Acids Research 43, 4517-4530.
96. Menon, S., Chi, H., Zhang, H., Deng, X. W., Flavell, R. A., and Wei, N. (2007). COP9 signalosome subunit 8 is essential for peripheral T cell homeostasis and antigen receptor-induced entry into the cell cycle from quiescence. Nat Immunol 8, 1236-1245.
97. Mu, J. Y., Zhuang, X. Y., Wang, Q. L., Jiang, H., Deng, Z. B., Wang, B. M., Zhang, L. F., Kakar, S., Jun, Y., Miller, D., and Zhang, H. G. (2014). Interspecies communication between plant and mouse gut host cells through edible plant derived exosome-like nanoparticles. Mol Nutr Food Res 58, 1561-1573.
98. Nilsson, J. A., Keller, U. B., Baudino, T. A., Yang, C., Norton, S., Old, J. A., Nilsson, L. M., Neale, G., Kramer, D. L., Porter, C. W., and Cleveland, J. L. (2005). Targeting ornithine decarboxylase in Myc-induced lymphomagenesis prevents tumor formation. Cancer Cell 7, 433-444.
99. Ouellette, A. J. (2010). Paneth cells and innate mucosal immunity. Curr Opin Gastroenterol 26, 547-553.
100. Pegg, A. E., and Casero, R. A., Jr. (2011). Current status of the polyamine research field. Methods Mol Biol 720, 3-35.
101. Peng, Z., Shen, Y., Feng, S., Wang, X., Chitteti, B. N., Vierstra, R. D., and Deng, X. W. (2003). Evidence for a physical association of the COP9 signalosome, the proteasome, and specific SCF E3 ligases in vivo. Curr Biol 13, R504-505.
102. Peterson, L. W., and Artis, D. (2014). Intestinal epithelial cells: regulators of barrier function and immune homeostasis. Nat Rev Immunol 14, 141-153.
103. Pickert, G., Neufert, C., Leppkes, M., Zheng, Y., Wittkopf, N., Wamtjen, M., Lehr, H. A., Birth, S., Weigmann, B., Wirtz, S., et al. (2009). STAT3 links IL-22 signaling in intestinal epithelial cells to mucosal wound healing. J Exp Med 206, 1465-1472.
104. Salzman, N. H., de Jong, H., Paterson, Y., Harmsen, H. J. M., Welling, G. W., and Bos, N. A. (2002). Analysis of 16S libraries of mouse gastrointestinal microflora reveals a large new group of mouse intestinal bacteria. Microbiology 148, 3651-3660.
105. Salzman, N. H., Hung, K., Haribhai, D., Chu, H., Karlsson-Sjoberg, J., Amir, E., Teggatz, P., Barman, M., Hayward, M., Eastwood, D., et al. (2010). Enteric defensins are essential regulators of intestinal microbial ecology. Nat Immunol 11, 76-83.
106. Schlierf, A., Altmann, E., Quancard, J., Jefferson, A. B., Assenberg, R., Renatus, M., Jones, M., Hassiepen, U., Schaefer, M., Kiffe, M., et al. (2016). Targeted inhibition of the COP9 signalosome for treatment of cancer. Nat Commun 7, 13166.
107. Schwechheimer, C., Serino, G., Callis, J., Crosby, W. L., Lyapina, S., Deshaies, R. J., Gray, W. M., Estelle, M., and Deng, X. W. (2001). Interactions of the COP9 signalosome with the E3 ubiquitin ligase SCFTIRI in mediating auxin response. Science 292, 1379-1382.
108. Song, M., Garrett, W. S., and Chan, A. T. (2015). Nutrients, foods, and colorectal cancer prevention. Gastroenterology 148, 1244-1260 e1216.
109. Su, H., Li, F., Ranek, M. J., Wei, N., and Wang, X. (2011a). COP9 signalosome regulates autophagosome maturation. Circulation 124, 2117-2128.
110. Su, H., Li, J., Menon, S., Liu, J., Kumarapeli, A. R., Wei, N., and Wang, X. (2011b). Perturbation of cullin deneddylation via conditional Csn8 ablation impairs the ubiquitin-proteasome system and causes cardiomyocyte necrosis and dilated cardiomyopathy in mice. Circ Res 108, 40-50.
111. Su, H., Li, J., Menon, S., Liu, J., Kumarapeli, A. R., Wei, N., and Wang, X. (2011c). Perturbation of Cullin Deneddylation via Conditional Csn8 Ablation Impairs the Ubiquitin-Proteasome System and Causes Cardiomyocyte Necrosis and Dilated Cardiomyopathy in Mice. Circulation Research 108, 40-50.
112. Timmons, J., Chang, E. T., Wang, J. Y., and Rao, J. N. (2012). Polyamines and Gut Mucosal Homeostasis. J Gastrointest Dig Syst 2.
113. Vaishnava, S., Behrendt, C. L., Ismail, A. S., Eckmann, L., and Hooper, L. V. (2008). Paneth cells directly sense gut commensals and maintain homeostasis at the intestinal host-microbial interface. Proceedings of the National Academy of Sciences 105, 20858-20863.
114. Vaishnava, S., Yamamoto, M., Severson, K. M., Ruhn, K. A., Yu, X., Koren, O., Ley, R., Wakeland, E. K., and Hooper, L. V. (2011). The Antibacterial Lectin Regllly Promotes the Spatial Segregation of Microbiota and Host in the Intestine. Science 334, 255-258.
115. Wallick, C. J., Gamper, I., Thome, M., Feith, D. J., Takasaki, K. Y., Wilson, S. M., Seki, J. A., Pegg, A. E., Byus, C. V., and Bachmann, A. S. (2005). Key role for p27Kip1, retinoblastoma protein Rb, and MYCN in polyamine inhibitor-induced G1 cell cycle arrest in MYCN-amplified human neuroblastoma cells. Oncogene 24, 5606-5618.
116. Wang, J. Y. (2007). Polyamines and mRNA stability in regulation of intestinal mucosal growth. Amino Acids 33, 241-252.
117. Wang, Y., Devereux, W., Stewart, T. M., and Casero, R. A., Jr. (2002a). Polyamine-modulated factor 1 binds to the human homologue of the 7a subunit of the Arabidopsis COP9 signalosome: implications in gene expression. Biochem J 366, 79-86.
118. Wang, Y. L., Devereux, W., Stewart, T. M., and Casero, R. A. (1999). Cloning and characterization of human polyamine-modulated factor-1, a transcriptional cofactor that regulates the transcription of the spermidine/spermine N-1-acetyltransferase gene. Journal of Biological Chemistry 274, 22095-22101.
119. Wang, Y. L., Devereux, W., Stewart, T. M., and Casero, R. A. (2002b). Polyamine-modulated factor 1 binds to the human homologue of the 7a subunit of the Arabidopsis COP9 signalosome: implications in gene expression. Biochem J 366, 79-86.
120. Wehkamp J, F. K., Herrlinger K R, Bevins C L, Stange E F. (2005). Mechanisms of disease: defensins in gastrointestinal diseases. Nat Clin Pract Gastroenterol Hepatol 2.
121. Wei, N., and Deng, X. W. (1998). Characterization and purification of the mammalian COP9 complex, a conserved nuclear regulator initially identified as a repressor of photomorphogenesis in higher plants. Photochem Photobiol 68, 237-241.
122. Wei, N., and Deng, X. W. (2003). THE COP9 SIGNALOSOME. Annual Review of Cell and Developmental Biology 19, 261-286.
123. Yang, X., Menon, S., Lykke-Andersen, K., Tsuge, T., Di, X., Wang, X., Rodriguez-Suarez, R. J., Zhang, H., and Wei, N. (2002). The COP9 signalosome inhibits p27(kip1) degradation and impedes G1-S phase progression via deneddylation of SCF Cull. Curr Biol 12, 667-672.
124. Zhao, R., Yeung, S. C., Chen, J., Iwakuma, T., Su, C. H., Chen, B., Qu, C., Zhang, F., Chen, Y. T., Lin, Y. L., et al. (2011). Subunit 6 of the COP9 signalosome promotes tumorigenesis in mice through stabilization of MDM2 and is upregulated in human cancers. J Clin Invest 121, 851-865.
125. Zhuang, X. Y., Deng, Z. B., Mu, J. Y., Zhang, L. F., Yan, J., Miller, D., Feng, W. K., McClain, C. J., and Zhang, H. G. (2015). Ginger-derived nanoparticles protect against alcohol-induced liver damage. J Extracell Vesicles 4.
126. Zou, T., Rao, J. N., Liu, L., Marasa, B. S., Keledjian, K. M., Zhang, A. H., Xiao, L., Bass, B. L., and Wang, J. Y. (2005). Polyamine depletion induces nucleophosmin modulating stability and transcriptional activity of p53 in intestinal epithelial cells. Am J Physiol Cell Physiol 289, C686-696.

It will be understood that various details of the presently disclosed subject matter can be changed without departing from the scope of the subject matter disclosed herein. Furthermore, the foregoing description is for the purpose of illustration only, and not for the purpose of limitation.

1. A method of treating intestinal inflammation, comprising administering to a subject in need thereof an effective amount of a broccoli-derived nanoparticle.

2. The method of claim 1, wherein the intestinal inflammation is colitis.

3. The method of claim 1, wherein the broccoli-derived nanoparticle is administered orally.

4. The method of claim 1, wherein administering the broccoli-derived nanoparticle increases an amount of adenosine monophosphate-activated protein kinase (AMPK) signaling in the subject.

5. The method of claim 1, wherein the broccoli-derived nanoparticle includes an effective amount of sulforaphane.

6. The method of claim 1, wherein administering the broccoli-derived nanoparticle reduces an amount of an inflammatory cytokine in the subject.

7. The method of claim 6, wherein the inflammatory cytokine is selected from the group consisting of interferon γ, tumor necrosis factor-α, and interleukin 17A.

8. The method of claim 1, wherein administering the broccoli-derived nanoparticle reduces an amount of dendritic cell activation and/or increases an amount of dendritic cell tolerance in the subject.

9. A method of treating a colon cancer, comprising administering to a subject in need thereof an effective amount of a broccoli-derived nanoparticle.

10. The method of claim 9, wherein administering the broccoli-derived nanoparticle decreases an amount of expression of COP9 signalsome subunit 8 (CSN8).

11. The method of claim 9, wherein administering the broccoli-derived nanoparticle reduces an amount of polyamine metabolism in an intestinal epithelial cell of the subject.

12. The method of claim 9, wherein administering the broccoli-derived nanoparticle reduces an amount of inflammation in the colon of the subject.

13. The method of claim 9, wherein administering the broccoli-derived nanoparticle increases an amount of an antimicrobial peptide in an intestinal epithelial cell of the subject.

14. The method of claim 9, wherein administering the broccoli-derived nanoparticle increases an amount of Bacteroidetes bacteria, reduces an amount of Actinobacteria bacteria, and/or reduces an amount of Proteobacteria bacteria present in the colon of the subject.

15. The method of claim 9, wherein administering the broccoli-derived nanoparticle reduces an amount of an inflammatory cytokine and/or reduces an amount of an inflammatory chemokine in the subject.

16. The method of claim 15, wherein the inflammatory cytokine is selected from the group consisting of interleukin 22, tumor necrosis factor-α, and interleukin 17A, and wherein the inflammatory chemokine is selected from the group consisting of CCL20, CXCL1, and CCL25.

17. The method of claim 9, wherein administering the broccoli-derived nanoparticle reduces an amount of rectal prolapse in the subject.

18. A pharmaceutical composition, comprising a broccoli-derived nanoparticle and a pharmaceutically-acceptable vehicle, carrier, or excipient.

19. The pharmaceutical composition of claim 18, wherein the broccoli-derived nanoparticle includes an effective amount of sulforaphane.

20. (canceled)
